(19) United States
(12) Patent Application Publication
(10) Pub. No.: US 2023/0414606 A1
(43) Pub. Date: Dec. 28, 2023

(54) METHODS OF TREATING PSYCHIATRIC DISORDERS IN OBESE PATIENTS WITH BREXPIPRAZOLE

(71) Applicant: Lake O'Hara LLC, Corona Del Mar, CA (US)

(72) Inventors: Sundar SRINIVASAN, Corona Del Mar, CA (US); Christina CHOW WALLEN, Seattle, WA (US)

(21) Appl. No.: 18/101,340

(22) Filed: Jan. 25, 2023

Related U.S. Application Data
(63) Continuation of application No. PCT/US2023/061104, filed on Jan. 23, 2023.
(60) Provisional application No. 63/302,328, filed on Jan. 24, 2022.

(51) Int. Cl. A61K 31/496; A61K 9/00; A61P 25/24; A61P 25/18

(52) U.S. Cl. CPC A61K 31/496; A61K 9/0053; A61P 25/24; A61P 25/18

(57) ABSTRACT

The present disclosure relates to methods of initiating brexpiprazole treatment in patients with schizophrenia or major depressive disorder. In embodiments, an increased dose (relative to the usual dose) is administered to said patients during treatment initiation. The present disclosure further relates to modified dosing regimens for patients that are obese and/or CYP2D6 poor metabolizers.

### CROSS-REFERENCE TO RELATED APPLICATIONS

This application is a continuation of International Application PCT/US2023/061104, filed Jan. 23, 2023, which claims the benefit of and priority to U.S. Provisional Application No. 63/302,328, filed Jan. 24, 2022, which is hereby incorporated by reference in its entirety.

### BACKGROUND

Brexpiprazole, also called REXULTI®, is an atypical antipsychotic used for treating major depressive disorder and schizophrenia. The mechanism of action of brexpiprazole in the treatment of major depressive disorder (MDD) and schizophrenia is unknown. However, the efficacy of brexpiprazole may result from partial agonist activity at serotonin 5-HT_{1A} and dopamine D2 receptors and antagonist activity at serotonin 5-HT_{2A} receptors.

The brexpiprazole (REXULTI®) Food and Drug Administration (FDA) label revised in March 2020 reflects the state-of-the art regarding the appropriate dosing for patients in need of brexpiprazole, and provides instructions for a brexpiprazole starting dose, recommended dose, and maximum dose with a titration timeline based on a patient's clinical response and tolerability. The FDA label also provides instructions to administer half of the usual dose to patients that are CYP2D6 poor metabolizers.

An ideal dosage regimen for brexpiprazole enables psychiatric patients to reach therapeutic levels of brexpiprazole as quickly as possible while avoiding side effects from brexpiprazole. One serious side effect of administering too much brexpiprazole is akathisia, a movement and mental distress disorder which is a state of agitation, distress, and restlessness. Akathisia rates in brexpiprazole patients have been shown to be dose-dependent, and increase as exposure to brexpiprazole increases.

It is not presently recognized in the art that the dosing regimen for brexpiprazole should be adjusted based on the body weight or obesity status of the patient, despite the fact that obesity and schizophrenia or depression are often comorbid conditions. While the REXULTI® label, for example, teaches that weight gain can be a side effect of treatment with brexpiprazole, or that being overweight is a risk factor for other side effects such as hyperglycemia, the label does not instruct any changes in dosing for obese or overweight patients compared to normal weight patients. Brexpiprazole dosing adjustments are only recommended based on the indication treated, hepatic or renal impairment status, or drug interactions with CYP2D6 inhibitors or CYP3A4 inhibitors or inducers.

### SUMMARY OF THE INVENTION

The inventors have discovered that the pharmacokinetics of brexpiprazole are substantively different in obese patients. Consequently, dosing changes are required when initiating treatment with brexpiprazole to achieve the same clinical response: effective treatment of schizophrenia and major depressive disorder. Prior to this invention, the standard of care left such obese patients untreated or undertreated, delaying resolution of their condition.

The invention addresses additional complexities and identifies modified dosing regimens that are critical to safely and effectively initiate treatment with brexpiprazole. In various embodiments, the present invention is directed to, inter alia, specific dose adjustments that avoid under-treatment but do not exceed known exposure levels that would expose patients to serious side effects. In various embodiments the present invention is directed to, inter alia, specific dosing regimens needed for different indications (schizophrenia or MDD) and for different CYP2D6 metabolizer status (extensive or poor).

In embodiments, the disclosure provides a method of initiating treatment of schizophrenia with brexpiprazole in an obese patient who is not a CYP2D6 poor metabolizer (i.e., a patient who is an extensive metabolizer; i.e., a patient classified as having a CYP2D6 enzyme phenotype with normal levels of CYP2D6 activity), comprising: (a) orally administering 1.125-2 mg brexpiprazole once daily on each of the first 4 days of brexpiprazole treatment; (b) orally administering 2.125-3.875 mg brexpiprazole once daily on each of the next 3 days following step (a); (c) orally administering 4.125-6 mg brexpiprazole on each of the next 7 days following step (b); and then (d) orally administering a recommended dose of 2-4 mg brexpiprazole once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 1.25-2 mg of brexpiprazole is administered in step (a). In embodiments, 1.25 mg of brexpiprazole is administered in step (a). In embodiments, 1.5 mg of brexpiprazole is administered in step (a). In embodiments, 2 mg of brexpiprazole is administered in step (a). In embodiments, 2.25-3.75 mg of brexpiprazole is administered in step (b). In embodiments, 1.5 mg of brexpiprazole is administered in step (a). In embodiments, 2.5 mg of brexpiprazole is administered in step (b). In embodiments, 1.5 mg of brexpiprazole is administered in step (a). In embodiments, 3 mg of brexpiprazole is administered in step (b). In embodiments, 4.25-6 mg of brexpiprazole is administered in step (c). In embodiments, 6 mg of brexpiprazole is administered in step (c). In embodiments, 5 mg of brexpiprazole is administered in step (c).

In embodiments, the disclosure provides a method of initiating treatment of schizophrenia with brexpiprazole in an obese patient who is not a CYP2D6 poor metabolizer, comprising: (a) orally administering 110-190% of the usual brexpiprazole dose of 1 mg once daily on each of the first 4 days of brexpiprazole treatment; (b) orally administering 110-190% of the usual brexpiprazole dose of 2 mg once daily on each of the next 3 days following step (a); (c) orally administering 110-190% of the usual brexpiprazole dose of 4 mg once daily on each of the next 7 days following step (b); and then (d) orally administering a recommended dose of 2-4 mg brexpiprazole once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 110%-175% of the usual brexpiprazole dose is administered in step (a). In embodiments, 125% of the usual brexpiprazole dose is administered in step (a). In embodiments, 150% of the usual brexpiprazole dose is administered in step (a). In embodiments, 110%475% of the usual brexpiprazole dose is administered in step (b). In embodiments, 125% of the usual brexpiprazole dose is administered in step (b). In embodiments, 150% of the usual brexpiprazole dose is administered in step (b). In embodiments, 110%-175% of the usual brexpiprazole dose is administered in step (b). In embodiments, 125% of the usual brexpiprazole dose is administered in step (c). In embodiments, 150% of the usual brexpiprazole dose is administered in step (c).

In embodiments, the disclosure provides a method of initiating treatment of schizophrenia with brexpiprazole in an obese patient who is not a CYP2D6 poor metabolizer, comprising: (a) orally administering 0.5-1.75 more than the usual brexpiprazole dose of 1 mg once daily on each of the first 4 days of brexpiprazole treatment; (b) orally administering 0.5-1.75 more than the usual brexpiprazole dose of 2 mg once daily on each of the next 3 days following step (a); (c) orally administering 0.5-1.75 more than the usual brexpiprazole dose of 4 mg once daily on each of the next 7 days following step (b); and then (d) orally administering a recommended dose of 2-4 mg brexpiprazole once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, about 1 mg more than the usual dose is administered is administered in each of steps (a), (b), and (c).

In embodiments, the recommended dose of brexpiprazole for treating schizophrenia in a patient who is not a CYP2D6 poor metabolizer is 2 mg/day. In embodiments, the recommended dose of brexpiprazole is 2.25 mg/day. In embodiments, the recommended dose of brexpiprazole is 2.5 mg/day. In embodiments, the recommended dose of brexpiprazole is 2.75 mg/day. In embodiments, the recommended dose of brexpiprazole is 3 mg/day. In embodiments, the recommended dose of brexpiprazole is 3.25 mg/day. In embodiments, the recommended dose of brexpiprazole is 3.5 mg/day. In embodiments, the recommended dose of brexpiprazole is 3.75 mg/day. In embodiments, the recommended dose of brexpiprazole is 4 mg/day.

In embodiments, the disclosure provides a method of initiating treatment of schizophrenia with brexpiprazole in an obese pediatric patient who is not a CYP2D6 poor metabolizer, comprising: (a) orally administering 0.625-1.125 mg brexpiprazole once daily on each of the first 4 days of brexpiprazole treatment; (b) orally administering 1.25-2.25 mg brexpiprazole once daily on each of the next 3 days following step (a); (c) orally administering 2.325-4 mg brexpiprazole on each of the next 7 days following step (b); and then (d) orally administering a recommended dose of 2-4 mg brexpiprazole once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg.

In embodiments, the disclosure provides a method of initiating treatment of schizophrenia with brexpiprazole in an obese pediatric patient who is not a CYP2D6 poor metabolizer, comprising: (a) orally administering 110-190% of the usual brexpiprazole dose of 0.5 mg once daily on each of the first 4 days of brexpiprazole treatment; (b) orally administering 110-190% of the usual brexpiprazole dose of 1 mg once daily on each of the next 3 days following step (a); (c) orally administering 110-190% of the usual brexpiprazole dose of 2 mg once daily on each of the next 7 days following step (b); and then (d) orally administering a recommended dose of 2-4 mg brexpiprazole once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 110%-175% of the usual brexpiprazole dose is administered in step (a). In embodiments, 125% of the usual brexpiprazole dose is administered in step (a). In embodiments, 150% of the usual brexpiprazole dose is administered in step (a).

In embodiments, the disclosure provides a method of initiating treatment of schizophrenia with brexpiprazole in an obese pediatric patient who is not a CYP2D6 poor metabolizer, comprising: (a) orally administering 0.5-1.75 more than the usual brexpiprazole dose of 0.5 mg once daily on each of the first 4 days of brexpiprazole treatment; (b) orally administering 0.5-1.75 more than the usual brexpiprazole dose of 1 mg once daily on each of the next 3 days following step (a); (c) orally administering 0.5-1.75 more than the usual brexpiprazole dose of 2 mg once daily on each of the next 7 days following step (b); and then (d) orally administering a recommended dose of 2-4 mg brexpiprazole once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, about 1 mg more than the usual dose is administered is administered in each of steps (a), (b), and (c). In embodiments, about 0.75 mg more than the usual dose is administered is administered in each of steps (a), (b), and (c).

In embodiments, the recommended dose of brexpiprazole for treating schizophrenia in a pediatric patient who is not a CYP2D6 poor metabolizer is 2 mg/day. In embodiments, the recommended dose of brexpiprazole is 2.25 mg/day. In embodiments, the recommended dose of brexpiprazole is 2.5 mg/day. In embodiments, the recommended dose of brexpiprazole is 2.75 mg/day. In embodiments, the recommended dose of brexpiprazole is 3 mg/day. In embodiments, the recommended dose of brexpiprazole is 3.25 mg/day. In embodiments, the recommended dose of brexpiprazole is 3.5 mg/day. In embodiments, the recommended dose of brexpiprazole is 3.75 mg/day. In embodiments, the recommended dose of brexpiprazole is 4 mg/day.

In embodiments, the disclosure provides method of initiating treatment of schizophrenia with brexpiprazole in an obese patient who is a CYP2D6 poor metabolizer, comprising: (a) orally administering 1.125-2 mg brexpiprazole once daily on each of the first 4 days of brexpiprazole treatment; (b) orally administering at least 2.125-3.875 mg brexpiprazole once daily on each of the next 3 days following step (a); (c) orally administering at least 4.125-7 mg brexpiprazole on each of the next 7 days following step (b); and then (d) orally administering 1-2 mg brexpiprazole once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 1.25-2 mg of brexpiprazole is administered in step (a). In embodiments, 1.25 mg of brexpiprazole is administered in step (a). In embodiments, 1.5 mg of brexpiprazole is administered in step (a). In embodiments, 2 mg of brexpiprazole is administered in step (a). In embodiments, 2.25-3.75 mg of brexpiprazole is administered in step (b). In embodiments, 2.5 mg of brexpiprazole is administered in step (b). In embodiments, 3 mg of brexpiprazole is administered in step (b). In embodiments, 4.25-6 mg of brexpiprazole is administered in step (c). In embodiments, 6 mg of brexpiprazole is administered in step (c). In embodiments, 5 mg of brexpiprazole is administered in step (c).

In embodiments, the disclosure provides a method of initiating treatment of schizophrenia with brexpiprazole in an obese patient who is a CYP2D6 poor metabolizer, comprising: (a) orally administering 110-190% of the usual brexpiprazole dose of 1 mg once daily on each of the first 4 days of brexpiprazole treatment; (b) orally administering 110-190% of the usual brexpiprazole dose of 2 mg once daily on each of the next 3 days following step (a); (c) orally administering 110-190% of the usual brexpiprazole dose of 4 mg once daily on each of the next 7 days following step (b); and then (d) orally administering 1-2 mg brexpiprazole once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 110%-175% of the usual brexpiprazole dose is administered in step (a). In embodiments, 125% of the usual brexpiprazole dose is administered in step (a). In embodiments, 150% of the usual brexpiprazole dose is administered in step (a). In embodiments, 110%-175% of the usual brexpiprazole dose is administered in step (b). In embodiments, 125% of the usual brexpiprazole dose is administered in step (b). In embodiments, 150% of the usual brexpiprazole dose is administered in step (b). In embodiments, 110%-175% of the usual brexpiprazole dose is administered in step (c). In embodiments, 125% of the usual brexpiprazole dose is administered in step (c). In embodiments, 150% of the usual brexpiprazole dose is administered in step (c).

In embodiments, the disclosure provides a method of initiating treatment of schizophrenia with brexpiprazole in an obese patient who is a CYP2D6 poor metabolizer, comprising: (a) orally administering 1-2 mg more than half the usual brexpiprazole dose of 1 mg once daily (e.g., the usual dose is 1 mg, half the usual dose is 0.5 mg, and step (a) is administering 1.5-2.5 mg once daily) on each of the first 4 days of brexpiprazole treatment; (b) orally administering 1-2 mg more than half the usual brexpiprazole dose of 1 mg once daily (e.g., administering 2-3 mg once daily) on each of the next 3 days following step (a); (c) orally administering 1-2 mg more than half the usual brexpiprazole dose of 2 mg once daily (e.g., administering 3-4 mg once daily) on each of the next 7 days following step (b); and then (d) orally administering 1-2 mg brexpiprazole once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, about 1.5 mg more than the usual dose is administered is administered in each of steps (a), (b), and (c).

In embodiments, an obese patient with schizophrenia who is a CYP2D6 poor metabolizer is treated with half of the recommended dose in step (d). In embodiments, the patient is administered 1 mg/day brexpiprazole in step (d). In embodiments, the patient is administered 1.25 mg/day. In embodiments, the patient is administered 1.5 mg/day brexpiprazole in step (d). In embodiments, the patient is administered 1.75 mg/day in step (d). In embodiments, the patient is administered 2 mg/day brexpiprazole in step (d).

In embodiments, the disclosure provides a method of initiating treatment of major depressive disorder with brexpiprazole in an obese patient who is not a CYP2D6 poor metabolizer, comprising: (a) orally administering 0.625-0.875 mg brexpiprazole once daily on each of the first 7 days of brexpiprazole treatment; (b) orally administering 1.125-1.875 mg brexpiprazole once daily on each of the next 7 days following step (a); (c) orally administering 2-2.875 mg brexpiprazole once daily on each of the next 7 days following step (b); and then (d) orally administering a recommended dose of brexpiprazole of 2-3 mg once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 0.625-0.75 mg of brexpiprazole is administered in step (a). In embodiments, 0.625 mg of brexpiprazole is administered in step (a). In embodiments, 0.75 mg of brexpiprazole is administered in step (a). In embodiments, 1.125-1.875 mg of brexpiprazole is administered in step (b). In embodiments, 1.25 mg of brexpiprazole is administered in step (b). In embodiments, 1.5 mg of brexpiprazole is administered in step (b). In embodiments, 2-2.5 mg of brexpiprazole is administered in step (c). In embodiments, 2 mg of brexpiprazole is administered in step (c). In embodiments, 2.25 mg of brexpiprazole is administered in step (c). In embodiments, 2.5 mg of brexpiprazole is administered in step (c).

In embodiments, the disclosure provides a method of initiating treatment of major depressive disorder with brexpiprazole in an obese patient who is not a CYP2D6 poor metabolizer, comprising: (a) orally administering 110-190% of the usual brexpiprazole dose of mg once daily on each of the first 7 days of brexpiprazole treatment; (b) orally administering 110-190% of the usual brexpiprazole dose of 1 mg once daily on each of the next 7 days following step (a); (c) orally administering 100-190% of the usual brexpiprazole dose of 2 mg once daily on each of the next 7 days following step (b); and then (d) orally administering a recommended dose of brexpiprazole of 2-3 mg once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 110%-175% of the usual brexpiprazole dose is administered in step (a). In embodiments, 125% of the usual brexpiprazole dose is administered in step (a). In embodiments, 150% of the usual brexpiprazole dose is administered in step (a). In embodiments, 110%475% of the usual brexpiprazole dose is administered in step (b). In embodiments, 125% of the usual brexpiprazole dose is administered in step (b). In embodiments, 150% of the usual brexpiprazole dose is administered in step (b). In embodiments, 100%-175% of the usual brexpiprazole dose is administered in step (c). In embodiments, 150% of the usual brexpiprazole dose is administered in step (c). In embodiments, 125% of the usual brexpiprazole dose is administered in step (c).

In embodiments, the disclosure provides a method of initiating treatment of major depressive disorder with brexpiprazole in an obese patient who is not a CYP2D6 poor metabolizer, comprising: (a) orally administering 0.125-0.75 mg more than the usual brexpiprazole dose of 0.5 mg once daily on each of the first 7 days of brexpiprazole treatment; (b) orally administering 0.125-0.75 mg more than of the usual brexpiprazole dose of 1 mg once daily on each of the next 7 days following step (a); (c) orally administering 0.125-0.75 mg more than the usual brexpiprazole dose of 2 mg once daily on each of the next 7 days following step (b); and then (d) orally administering a recommended dose of brexpiprazole of 2-3 mg once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 0.5 mg more than the usual brexpiprazole dose is administered in each of steps (a), (b), and (c).

In embodiments, the recommended dose of brexpiprazole for major depressive disorder with brexpiprazole in a patient who is not a CYP2D6 poor metabolizer is 2 mg/day. In embodiments, the recommended dose of brexpiprazole is 2.25 mg/day. In embodiments, the recommended dose of brexpiprazole is 2.5 mg/day. In embodiments, wherein the recommended dose of brexpiprazole is 2.75 mg/day. In embodiments, wherein the recommended dose of brexpiprazole is 3 mg/day.

In embodiments, the disclosure provides a method of initiating treatment of major depressive disorder with brexpiprazole in an obese patient who is not a CYP2D6 poor metabolizer, comprising: (a) orally administering 1.125-1.875 mg brexpiprazole once daily on each of the first 7 days of brexpiprazole treatment; (b) orally administering 2.125-3.875 mg brexpiprazole once daily on each of the next 7 days following step (a); (c) orally administering 2-4 mg brexpiprazole once daily on each of the next 7 days following step (b); and then (d) orally administering a recommended dose of brexpiprazole of 2-3 mg once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 1.125-1.75 mg of brexpiprazole is administered in step (a). In embodiments, 1.25 mg of brexpiprazole is administered in step (a). In embodiments, wherein 1.5 mg of brexpiprazole is administered in step (a). In embodiments, wherein 2.125-3.5 mg of brexpiprazole is administered in step (b). In embodiments, 2.5 mg of brexpiprazole is administered in step (b). In embodiments, 3 mg of brexpiprazole is administered in step (b). In embodiments, 2-3 mg of brexpiprazole is administered in step (c). In embodiments, 2 mg of brexpiprazole is administered in step (c). In embodiments, 2.5 mg of brexpiprazole is administered in step (c).

In embodiments, the disclosure provides a method of initiating treatment of major depressive disorder with brexpiprazole in an obese patient who is not a CYP2D6 poor metabolizer, comprising: (a) orally administering 110-190% of the usual brexpiprazole dose of 1 mg once daily on each of the first 7 days of brexpiprazole treatment; (b) orally administering 110-190% of the usual brexpiprazole dose of 1 mg once daily on each of the next 7 days following step (a); (c) orally administering 100-190% mg of the usual brexpiprazole dose of 2 mg once daily on each of the next 7 days following step (b); and then (d) orally administering a recommended dose of brexpiprazole of 2-3 mg once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 110%-175% of the usual brexpiprazole dose is administered in step (a). In embodiments, 125% of the usual brexpiprazole dose is administered in step (a). In embodiments, 150% mg of the usual brexpiprazole dose is administered in step (a). In embodiments, 110%-175% of the usual brexpiprazole dose is administered in step (b). In embodiments, 125% of the usual brexpiprazole dose is administered in step (b). In embodiments, 150% of the usual brexpiprazole dose is administered in step (b). In embodiments, 100%-175% of the usual brexpiprazole dose is administered in step (c). In embodiments, the usual brexpiprazole dose is administered in step (c). In embodiments, 125% of the usual brexpiprazole dose is administered in step (c). In embodiments, the recommended dose of brexpiprazole is 2 mg/day.

In embodiments, the disclosure provides a method of initiating treatment of major depressive disorder with brexpiprazole in an obese patient who is not a CYP2D6 poor metabolizer, comprising: (a) orally administering 0.125-0.75 mg more than the usual brexpiprazole dose of 1 mg once daily on each of the first 7 days of brexpiprazole treatment; (b) orally administering 0.125-0.75 mg more than of the usual brexpiprazole dose of 2 mg once daily on each of days 8-14 following step (a); (c) orally administering 0.125-0.75 mg more than the usual brexpiprazole dose of 2-3 mg once daily on each of days 15-21 following step (b); and then (d) orally administering a recommended dose of brexpiprazole of 2-3 mg once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, mg more than the usual brexpiprazole dose is administered in each of steps (a), (b), and (c).

In embodiments, the recommended dose for treating major depressive disorder with brexpiprazole in an obese patient who is not a CYP2D6 poor metabolizer is 2 mg/day. In embodiments, the recommended dose is 2.25 mg/day. In embodiments, the recommended dose of brexpiprazole is 2.5 mg/day. In embodiments, the recommended dose of brexpiprazole is 2.75 mg/day. In embodiments, wherein the recommended dose of brexpiprazole is 3 mg/day.

A method of initiating treatment of major depressive disorder with brexpiprazole in an obese patient who is a CYP2D6 poor metabolizer, comprising: (a) orally administering 0.625-mg brexpiprazole once daily on each of the first 7 days of brexpiprazole treatment; (b) orally administering 1-1.875 mg brexpiprazole once daily on each of the next 7 days following step (a); orally administering 1-3 mg brexpiprazole once daily on each of the next 7 days following step (b); and then (d) orally administering 1-1.5 mg once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 0.625-0.75 mg of brexpiprazole is administered in step (a). In embodiments, 0.625 mg of brexpiprazole is administered in step (a). In embodiments, 0.75 mg of brexpiprazole is administered in step (a). In embodiments, 1-1.875 mg of brexpiprazole is administered in step (b). In embodiments, 1 mg of brexpiprazole is administered in step (b). In embodiments, 1.25 mg of brexpiprazole is administered in step (b). In embodiments, 1.5 mg of brexpiprazole is administered in step (b). In embodiments, 1-2.5 mg of brexpiprazole is administered in step (c). In embodiments, 1 mg of brexpiprazole is administered in step (c). In embodiments, 1.5 mg of brexpiprazole is administered in step (c). In embodiments, 2.5 mg of brexpiprazole is administered in step (c).

In embodiments, the disclosure provides a method of initiating treatment of major depressive disorder with brexpiprazole in an obese patient who is a CYP2D6 poor metabolizer, comprising: (a) orally administering 110-190% of the usual brexpiprazole dose of 0.5 mg once daily on each of the first 7 days of brexpiprazole treatment; (b) orally administering 110-190% of the usual brexpiprazole dose of 1 mg once daily on each of the next 7 days following step (a); (c) orally administering 50%-190% mg of the usual brexpiprazole dose of 2 mg once daily on each of the next 7 days following step (b); and then (d) orally administering 1-1.5 mg once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 110%-175% mg of the usual brexpiprazole dose is administered in step (a). In embodiments, 125% mg of the usual brexpiprazole dose is administered in step (a). In embodiments, 150% mg of the usual brexpiprazole dose is administered in step (a). In embodiments, 110%-175% mg of the usual brexpiprazole dose is administered in step (b). In embodiments, 125% mg of the usual brexpiprazole dose is administered in step (b). In embodiments, 150% mg of the usual brexpiprazole dose is administered in step (b). In embodiments, 50%-175% mg of the usual brexpiprazole dose is administered in step (c). In embodiments, 50% of the usual brexpiprazole dose is administered in step (c). In embodiments, 125% mg of the usual brexpiprazole dose is administered in step (c). In embodiments, 150% mg of the usual brexpiprazole dose is administered in step (c).

In embodiments, the disclosure provides a method of initiating treatment of major depressive disorder with brexpiprazole in an obese patient who is a CYP2D6 poor metabolizer, comprising: (a) orally administering 0.125-0.75 mg more than half of the usual dose of 0.5 mg once daily (e.g., administering 0.375-1 mg once daily) on each of the first 7 days of brexpiprazole treatment; (b) orally administering 0.125-0.75 mg more than half of the usual brexpiprazole dose of 1.0 mg once daily (e.g., administering 0.675-1.25 mg once daily) on each of the next 7 days following step (a); (c) orally administering 0.125-0.75 mg more than half of the usual brexpiprazole dose of 2-3 mg once daily (e.g., administering 1.175-2.25 mg once daily) on each of the next 7 days following step (b); and then (d) orally administering 1-1.5 mg once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 0.5 mg more than half the usual dose in each of steps (a), (b), and (c).

In embodiments, obese CYP2D6 poor metabolizers with major depressive disorder are administered half of the recommended dose in step (d). In embodiments, 1 mg/day is administered in step (d). In embodiments, 1.25 mg/day is administered in step (d). In embodiments, 1.5 mg/day is administered in step (d).

In embodiments, the disclosure provides a method of initiating treatment of major depressive disorder with brexpiprazole in an obese patient who is a CYP2D6 poor metabolizer, comprising: (a) orally administering 1.125-1.875 mg brexpiprazole once daily on each of the first 7 days of brexpiprazole treatment; (b) orally administering 2-3.5 mg brexpiprazole once daily on each of the next 7 days following step (a); (c) orally administering 1-3 mg brexpiprazole once daily on each of the next 7 days following step (b); and then (d) orally administering 1-1.5 mg brexpiprazole once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 1.125-1.75 mg of brexpiprazole is administered in step (a). In embodiments, 1.25 mg of brexpiprazole is administered in step (a). In embodiments, 1.5 mg of brexpiprazole is administered in step (a). In embodiments, 2-3 mg of brexpiprazole is administered in step (b). In embodiments, 2 mg of brexpiprazole is administered in step (b). In embodiments, 2.5 mg of brexpiprazole is administered in step (b). In embodiments, 3 mg of brexpiprazole is administered in step (b). In embodiments, 1-2.5 mg of brexpiprazole is administered in step (c). In embodiments, 1 mg of brexpiprazole is administered in step (c). In embodiments, 2 mg of brexpiprazole is administered in step (c). In embodiments, 2.5 mg of brexpiprazole is administered in step (c).

In embodiments, the disclosure provides a method of initiating treatment of major depressive disorder with brexpiprazole in an obese patient who is a CYP2D6 poor metabolizer, comprising: (a) orally administering 110-190% of the usual brexpiprazole dose of 1 mg once daily on each of the first 7 days of brexpiprazole treatment; (b) orally administering 110-190% of the usual brexpiprazole dose of 1 mg once daily on each of the next 7 days following step (a); (c) orally administering 100-190% mg of the usual brexpiprazole dose of 2 mg once daily on each of the next 7 days following step (b); and then (d) orally administering a recommended dose of brexpiprazole of 1.5 mg once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 110%475% mg of the usual brexpiprazole dose is administered in step (a). In embodiments, 125% mg of the usual brexpiprazole dose is administered in step (a). In embodiments, 150% mg of the usual brexpiprazole dose is administered in step (a). In embodiments, 110%-175% mg of the usual brexpiprazole dose is administered in step (b). In embodiments, 125% mg of the usual brexpiprazole dose is administered in step (b). In embodiments, 150% mg of the usual brexpiprazole dose is administered in step (b). In embodiments, 100%-175% mg of the usual brexpiprazole dose is administered in step (c). In embodiments, the usual brexpiprazole dose is administered in step (c). In embodiments, 125% mg of the usual brexpiprazole dose is administered in step (c). In embodiments, 150% mg of the usual brexpiprazole dose is administered in step (c).

In embodiments, the disclosure provides a method of initiating treatment of major depressive disorder with brexpiprazole in an obese patient who is a CYP2D6 poor metabolizer, comprising: (a) orally administering 0.5-1.5 mg more than half of the usual dose of 1 mg once daily (e.g., administering 1-2 mg once daily) on each of the first 7 days of brexpiprazole treatment; (b) orally administering 0.5-1.5 mg more than half of the usual brexpiprazole dose of 2 mg once daily (e.g., administering 1.5-2.5 mg once daily) on each of the next 7 days following step (a); (c) orally administering 0.5-1.5 mg more than half of the usual brexpiprazole dose of 2-3 mg once daily (e.g., administering 1.5-3 mg once daily) on each of the next 7 days following step (b); and then (d) orally administering 1-1.5 mg once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 1 mg more than half the usual brexpiprazole dose is administered in each of steps (a), (b), and (c).

In embodiments, obese CYP2D6 poor metabolizers with major depressive disorder are administered half of the recommended dose in step (d). In embodiments, the patient is administered 1 mg/day in step (d). In embodiments, the patient is administered 1.25 mg/day in step (d). In embodiments, the patient is administered is 1.5 mg/day in step (d).

Applicant also surprisingly discovered that normal-weight CYP2D6 PM patients do not reach therapeutic levels as quickly as normal-weight CYP2D6 EM (FIGS. 3A-3C, 6A-6C, and 9A-9C). Administering brexpiprazole according to a modified dosage regimen provided herein enables normal-weight CYP2D6 PM patients to approach therapeutic levels of brexpiprazole as quickly as normal-weight CYP2D6 EM patients (FIGS. 3A-3C, 6A-6C, and 9A-9C)

In embodiments, the disclosure provides a method of initiating treatment of schizophrenia with brexpiprazole in a normal-weight patient who is a CYP2D6 poor metabolizer, comprising: (a) administering 0.625-1.325 mg brexpiprazole once daily on each of the first 4 days of brexpiprazole treatment; (b) administering 1.5-2 mg brexpiprazole once daily on each of the next 3 days following step (a); and then (c) 2.5-3.5 mg brexpiprazole once daily on each of the next 3 days following step (b); and then (d) administering 1-2 mg brexpiprazole once daily thereafter, wherein the normal-weight patient has at least one of the following characteristics: (i) BMI less than about 35 kg/m²; (ii) % IBW less than about 150%; (iii) waist size less than about 42 inches; (iv) % body fat less than about 40%; (v) % android body fat less than about 40%; (vi) % gynoid body fat less than about 40%; or (vii) total body fat less than about 40 kg. In embodiments, 0.75-1.25 mg of brexpiprazole is administered in step (a). In embodiments, 0.75 mg of brexpiprazole is administered in step (a). In embodiments, 1.25 mg of brexpiprazole is administered in step (a). In embodiments, 1.5-1.75 mg of brexpiprazole is administered in step (b). In embodiments, 1.5 mg of brexpiprazole is administered in step (b). In embodiments, 1.75 mg of brexpiprazole is administered in step (b). In embodiments, 2.75-3.25 mg of brexpiprazole is administered in step (c). In embodiments, 2.75 mg of brexpiprazole is administered in step (c). In embodiments, 3 mg of brexpiprazole is administered in step (c).

In embodiments, the disclosure provides a method of initiating treatment of schizophrenia with brexpiprazole in a normal-weight patient who is a CYP2D6 poor metabolizer, comprising: (a) orally administering 60-90% of the usual brexpiprazole dose of 1 mg once daily on each of the first 4 days of brexpiprazole treatment; (b) orally administering 60-90% of the usual brexpiprazole dose of 2 mg once daily on each of the next 3 days following step (a); (c) orally administering 60-90% of the usual brexpiprazole dose of 4 mg once daily on each of the next 7 days following step (b); and then (d) orally administering a recommended dose of 1-2 mg brexpiprazole once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 75% of the usual dose is administered in steps (a), (b), and (c).

In embodiments, the disclosure provides a method of initiating treatment of schizophrenia with brexpiprazole in a normal-weight patient who is a CYP2D6 poor metabolizer, comprising: (a) orally administering 0.5-1 mg more than half the usual brexpiprazole dose of 1 mg once daily on each of the first 4 days of brexpiprazole treatment; (b) orally administering 0.5-1 mg more than half the usual brexpiprazole dose of 2 mg once daily on each of the next 3 days following step (a); (c) orally administering 0.5-1 mg more than half the usual brexpiprazole dose of 4 mg once daily on each of the next 7 days following step (b); and then (d) orally administering 1-2 mg brexpiprazole once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 0.75 mg more than half the usual dose is administered in steps (a), (b), and (c).

In embodiments, 1 mg/day of brexpiprazole is administered in step (d). In embodiments, 1.25 mg/day of brexpiprazole is administered in step (d). In embodiments, 1.5 mg/day of brexpiprazole is administered in step (d). In embodiments, 1.75 mg/day of brexpiprazole is administered in step (d). In embodiments, wherein 2 mg/day of brexpiprazole is administered in step (d).

In embodiments, the disclosure provides a method of initiating adjunctive treatment of major depressive disorder with brexpiprazole in a normal-weight patient who is a CYP2D6 poor metabolizer, comprising: (a) administering 0.375 mg brexpiprazole once daily on each of the first 7 days of brexpiprazole treatment; (b) administering 0.625-0.875 mg of brexpiprazole once daily on each of the next 7 days following step (a); (c) administering 1.125-1.75 mg of brexpiprazole once daily on each of the next 7 days following step (b); (d) administering 1-1.5 mg of brexpiprazole once daily thereafter; wherein the normal-weight patient has at least one of the following characteristics: (i) BMI less than about 35 kg/m 2; (ii) % IBW less than about 150%; (iii) waist size less than about 42 inches; (iv) % body fat less than about 40%; (v) % android body fat less than about 40%; (vi) % gynoid body fat less than about 40%; or (vii) total body fat less than about 40 kg. In embodiments, 0.75 mg of brexpiprazole is administered in step (b). In embodiments, 1.25 mg of brexpiprazole is administered in step (c). In embodiments, 1.5 mg of brexpiprazole is administered in step (c).

In embodiments, the disclosure provides a method of initiating adjunctive treatment of major depressive disorder with brexpiprazole in a normal-weight patient who is a CYP2D6 poor metabolizer, comprising: (a) orally administering 60-90% of the usual brexpiprazole dose of 0.5 mg once daily on each of the first 7 days of brexpiprazole treatment; (b) orally administering 60-90% of the usual brexpiprazole dose of 1 mg once daily on each of the next 7 days following step (a); (c) orally administering 60-90% of the usual brexpiprazole dose of 2 mg once daily on each of the next 7 days following step (b); and then (d) orally administering a recommended dose of 1-1.5 mg brexpiprazole once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 75% of the usual dose is administered in steps (a), (b), and (c).

In embodiments, the disclosure provides a method of initiating adjunctive treatment of major depressive disorder with brexpiprazole in a normal-weight patient who is a CYP2D6 poor metabolizer, comprising: (a) orally administering 0.125-0.75 mg more than half the usual brexpiprazole dose of 0.5 mg once daily on each of the first 7 days of brexpiprazole treatment; (b) orally administering 0.125-0.75 mg more than half the usual brexpiprazole dose of 1 mg once daily on each of the next 7 days following step (a); (c) orally administering 0.125-0.75 mg more than half the usual brexpiprazole dose of 2 mg once daily on each of the next 7 days following step (b); and then (d) orally administering a recommended dose of 1-1.5 mg brexpiprazole once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 0.25 mg more than the usual dose is administered in steps (a), (b), and (c).

In embodiments, the disclosure provides a method of initiating adjunctive treatment of major depressive disorder with brexpiprazole in a normal-weight patient who is a CYP2D6 poor metabolizer, comprising: (a) administering 0.75 mg of brexpiprazole once daily on each of the first 7 days of brexpiprazole treatment; (b) administering 1.25-2 mg of brexpiprazole once daily on each of the next 7 days following step (a); (c) administering 1.5-3 mg of brexpiprazole once daily on each of the next 7 days following step (b); (d) administering 1-1.5 mg of brexpiprazole once daily thereafter; wherein the normal-weight patient has at least one of the following characteristics: (i) BMI less than about 35 kg/m 2; (ii) % IBW less than about 150%; (iii) waist size less than about 42 inches; (iv) % body fat less than about 40%; (v) % android body fat less than about 40%; (vi) % gynoid body fat less than about 40%; or (vii) total body fat less than about kg. In embodiments, 1.5 mg of brexpiprazole is administered in step (b). In embodiments, 1.5 mg of brexpiprazole is administered in step (c). In embodiments, 3 mg of brexpiprazole is administered in step (c). In embodiments, 1 mg/day of brexpiprazole is administered in step (d). In embodiments, 1.25 mg/day of brexpiprazole is administered in step (d). In embodiments, 1.5 mg/day of brexpiprazole is administered in step (d).

In embodiments, the disclosure provides a method of initiating adjunctive treatment of major depressive disorder with brexpiprazole in a normal-weight patient who is a CYP2D6 poor metabolizer, comprising: (a) orally administering 60-90% of the usual brexpiprazole dose of 1 mg once daily on each of the first 7 days of brexpiprazole treatment; (b) orally administering 60-90% of the usual brexpiprazole dose of 2 mg once daily on each of the next 7 days following step (a); (c) orally administering 60-90% of the usual brexpiprazole dose of 2-3 mg once daily on each of the next 7 days following step (b); and then (d) orally administering 1-1.5 mg brexpiprazole once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 75% of half the usual dose is administered in steps (a), (b), and (c).

In embodiments, the disclosure provides a method of initiating adjunctive treatment of major depressive disorder with brexpiprazole in a normal-weight patient who is a CYP2D6 poor metabolizer, comprising: (a) orally administering 0.125-0.75 mg more than half the usual brexpiprazole dose of 1 mg once daily on each of the first 7 days of brexpiprazole treatment; (b) orally administering 0.125-0.75 mg more than half the usual brexpiprazole dose of 2 mg once daily on each of the next 7 days following step (a); (c) orally administering 0.125-0.75 mg more than half the usual brexpiprazole dose of 2-3 mg once daily on each of the next 7 days following step (b); and then (d) orally administering 1-1.5 mg brexpiprazole once daily thereafter; wherein the obese patient has one or more of the following characteristics: (i) BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; or (vii) total body fat greater than about 40 kg. In embodiments, 75% of half the usual dose is administered in steps (a), (b), and (c). In embodiments, 0.5 mg more than half the usual dose is administered in steps (a), (b), and (c).

### BRIEF DESCRIPTION OF THE FIGURES

FIG. 1A shows the mean area under the blood plasma concentration curve from time 0 to 24 hours (AUC₂₄) of brexpiprazole in normal-weight CYP2D6 EM (“Expected AUC”) and obese CYP2D6 PM patients with schizophrenia that are treated according to the brexpiprazole FDA label for 28 days. Obese CYP2D6 PM patients treated according to the brexpiprazole FDA label take longer to reach therapeutic concentrations of brexpiprazole than normal-weight CYP2D6 EM patients, and may not reach therapeutic concentrations at all in the first 28 days of brexpiprazole administration.

FIG. 1B shows the AUC₂₄ of brexpiprazole in obese CYP2D6 PM patients with schizophrenia that are treated according to the modified dosing regimens disclosed herein (e.g., Table F, Modified Dosing Regimen 3.1 and 4.1). Obese CYP2D6 PM patients treated according to the disclosed regimens have similar AUC₂₄ during treatment initiation and reach therapeutic concentrations that are similar to normal-weight CYP2D6 EM patients (“Expected AUC”) that are treated according to the brexpiprazole FDA label.

FIG. 1C shows the maximum blood plasma concentrations (Cₘₐₓ) of brexpiprazole in normal-weight CYP2D6 EM (“Expected Cₘₐₓ”) and obese CYP2D6 PM patients with schizophrenia that are treated according to the brexpiprazole FDA label over 28 days. Obese CYP2D6 PM treated according to the brexpiprazole FDA label patients take longer to reach therapeutic concentrations of brexpiprazole than normal-weight CYP2D6 EM patients (“Expected Cₘₐₓ”), and may not reach therapeutic concentrations at all in the first 28 days of brexpiprazole administration.

FIG. 1D shows the Cₘₐₓ of brexpiprazole in obese CYP2D6 PM patients with schizophrenia that are treated according to the modified dosing regimens disclosed herein (e.g., Table F, Modified Dosing Regimen 3.1 and 4.1). Obese CYP2D6 PM patients treated according to the disclosed regimens have similar Cₘₐₓ during treatment initiation and reach therapeutic concentrations that are similar to normal-weight CYP2D6 EM patients (“Expected Cₘₐₓ”) that are treated according to the brexpiprazole FDA label.

FIG. 1E shows the minimum blood plasma concentrations (Cram) of brexpiprazole in normal-weight CYP2D6 EM (“Expected Cₘᵢₙ”) and obese CYP2D6 PM patients with schizophrenia that are treated according to the brexpiprazole FDA label over 28 days. Obese CYP2D6 PM patients treated according to the brexpiprazole FDA label take longer to reach therapeutic concentrations of brexpiprazole than normal-weight CYP2D6 EM patients, and may not reach therapeutic concentrations at all in the first 28 days of brexpiprazole administration.

FIG. 1F shows the Cₘᵢₙ of brexpiprazole in obese CYP2D6 PM patients with schizophrenia that are treated according to the modified dosing regimens disclosed herein (e.g., Table F, Modified Dosing Regimen 3.1 and 4.1). Obese CYP2D6 PM patients treated according to the disclosed regimens have similar Cₘᵢₙ during treatment initiation and reach therapeutic concentrations that are similar to normal-weight CYP2D6 EM patients that are treated according to the brexpiprazole FDA label.

FIG. 2A shows the AUC₂₄ of brexpiprazole in normal-weight CYP2D6 EM (“Expected AUC₂₄”) and obese CYP2D6 EM patients with schizophrenia that are treated according to the brexpiprazole FDA label, and obese CYP2D6 EM patients treated according to the disclosure (e.g., Table F, Modified Dosing Regimen 1.1 and 2.1). Obese CYP2D6 EM patients treated according to the brexpiprazole FDA label take longer to reach therapeutic concentrations of brexpiprazole than normal-weight CYP2D6 EM patients, and may not reach therapeutic concentrations at all in the first 28 days of brexpiprazole administration. Obese CYP2D6 EM patients treated according to the disclosed regimens have similar AUC₂₄ during treatment initiation and reach therapeutic concentrations that are similar to normal-weight CYP2D6 EM patients that are treated according to the brexpiprazole FDA label.

FIG. 2B shows the Cₘₐₓ of brexpiprazole in normal-weight CYP2D6 EM (“Expected Cₘₐₓ”) and obese CYP2D6 EM patients with schizophrenia that are treated according to the brexpiprazole FDA label, and obese CYP2D6 EM patients treated according to the disclosure (e.g., Table F, Modified Dosing Regimen 1.1 and 2.1). Obese CYP2D6 EM patients treated according to the brexpiprazole FDA label take longer to reach therapeutic concentrations of brexpiprazole than normal-weight CYP2D6 EM patients, and may not reach therapeutic concentrations at all in the first 28 days of brexpiprazole administration. Obese CYP2D6 EM patients treated according to the disclosed regimens have similar Cₘₐₓ during treatment initiation and reach therapeutic concentrations that are similar to normal-weight CYP2D6 EM patients that are treated according to the brexpiprazole FDA label.

FIG. 2C shows the Cram of brexpiprazole in normal-weight CYP2D6 EM (“Expected Cₘᵢₙ”) and obese CYP2D6 EM patients with schizophrenia that are treated according to the brexpiprazole FDA label, and obese CYP2D6 EM patients treated according to the disclosure (e.g., Table F, Modified Dosing Regimen 1.1 and 2.1). Obese CYP2D6 EM patients treated according to the brexpiprazole FDA label take longer to reach therapeutic concentrations of brexpiprazole than normal-weight CYP2D6 EM patients, and may not reach therapeutic concentrations at all in the first 28 days of brexpiprazole administration. Obese CYP2D6 EM patients treated according to the disclosed regimens have similar Cₘᵢₙ during treatment initiation and reach therapeutic concentrations that are similar to normal-weight CYP2D6 EM patients that are treated according to the brexpiprazole FDA label.

FIG. 3A shows the AUC₂₄ of brexpiprazole in normal-weight CYP2D6 EM (“Expected AUC₂₄”) and normal-weight CYP2D6 PM patients with schizophrenia that are treated according to the brexpiprazole FDA label, and normal-weight CYP2D6 PM patients treated according to the disclosure (e.g., Table F, Modified Dosing Regimen 9.1). Normal-weight CYP2D6 PM patients treated according to the brexpiprazole FDA label take longer to reach therapeutic concentrations of brexpiprazole than normal-weight CYP2D6 EM patients, and may not reach therapeutic concentrations at all in the first 28 days of brexpiprazole administration. Normal-weight CYP2D6 PM patients treated according to the disclosed regimens have similar AUC₂₄ during treatment initiation and reach therapeutic concentrations that are similar to normal-weight CYP2D6 EM patients that are treated according to the brexpiprazole FDA label.

FIG. 3B shows the Cₘₐₓ of brexpiprazole in normal-weight CYP2D6 EM (“Expected Cₘₐₓ”) and normal-weight CYP2D6 PM patients with schizophrenia that are treated according to the brexpiprazole FDA label, and normal-weight CYP2D6 PM patients treated according to the disclosure (e.g., Table F, Modified Dosing Regimen 9.1). Normal-weight CYP2D6 PM patients treated according FDA brexpiprazole label take longer to reach therapeutic concentrations of brexpiprazole than normal-weight CYP2D6 EM patients, and may not reach therapeutic concentrations at all in the first 28 days of brexpiprazole administration. Normal-weight CYP2D6 PM patients treated according to the disclosed regimens have similar Cₘₐₓ during treatment initiation and reach therapeutic concentrations that are similar to normal-weight CYP2D6 EM patients that are treated according to the brexpiprazole FDA label.

FIG. 3C shows the Cₘᵢₙ of brexpiprazole in normal-weight CYP2D6 EM (“Expected Cₘᵢₙ”) and normal-weight CYP2D6 PM patients with schizophrenia that are treated according to the brexpiprazole FDA label, and normal-weight CYP2D6 PM patients treated according to the disclosure (e.g., Table F, Modified Dosing Regimen 9.1). Normal-weight CYP2D6 PM patients treated according to the FDA label take longer to reach therapeutic concentrations of brexpiprazole than normal-weight CYP2D6 EM patients, and may not reach therapeutic concentrations at all in the first 28 days of brexpiprazole administration. Normal-weight CYP2D6 PM patients treated according to the disclosed regimens have similar Cₘᵢₙ during treatment initiation and reach therapeutic concentrations that are similar to normal-weight CYP2D6 EM patients that are treated according to the brexpiprazole FDA label.

FIG. 4A shows the mean AUC₂₄ of brexpiprazole in normal-weight CYP2D6 EM treated according to the brexpiprazole label, with a starting dose of 0.5 mg (“Expected AUC₂₄”), obese CYP2D6 PM patients with major depressive disorder (MDD) that are treated according to the brexpiprazole FDA label, with a starting dose of 0.25 mg, and obese CYP2D6 PM patients treated according to the disclosure (e.g., Table G, Modified Dosing Regimen E.1 and F.1). Obese CYP2D6 PM patients treated according to the brexpiprazole FDA label take longer to reach therapeutic concentrations of brexpiprazole than normal-weight CYP2D6 EM patients, and may not reach therapeutic concentrations at all in the first 28 days of brexpiprazole administration. Obese CYP2D6 PM patients treated according to the disclosed regimens have similar AUC₂₄ during treatment initiation and reach therapeutic concentrations that are similar to normal-weight CYP2D6 EM patients that are treated according to the brexpiprazole FDA label.

FIG. 4B shows the Cₘₐₓ of brexpiprazole in normal-weight CYP2D6 EM treated according to the brexpiprazole label, with a starting dose of 0.5 mg (“Expected Cₘₐₓ”), obese CYP2D6 PM patients with major depressive disorder (MDD) that are treated according to the brexpiprazole FDA label, with a starting dose of 0.25 mg, and obese CYP2D6 PM patients treated according to the disclosure (e.g., Table G, Modified Dosing Regimen E.1 and F.1). Obese CYP2D6 PM patients treated according to the brexpiprazole FDA label take longer to reach therapeutic concentrations of brexpiprazole than normal-weight CYP2D6 EM patients, and may not reach therapeutic concentrations at all in the first 28 days of brexpiprazole administration. Obese CYP2D6 PM patients treated according to the disclosed regimens have similar Cₘₐₓ during treatment initiation and reach therapeutic concentrations that are similar to normal-weight CYP2D6 EM patients that are treated according to the brexpiprazole FDA label.

FIG. 4C of brexpiprazole in normal-weight CYP2D6 EM treated according to the brexpiprazole label, with a starting dose of 0.5 mg (“Expected AUC₂₄”), obese CYP2D6 PM patients with major depressive disorder (MDD) that are treated according to the brexpiprazole FDA label, with a starting dose of 0.25 mg, and obese CYP2D6 PM patients treated according to the disclosure (e.g., Table G, Modified Dosing Regimen E.1 and F.1). Obese CYP2D6 PM patients treated according to the brexpiprazole FDA label take longer to reach therapeutic concentrations of brexpiprazole than normal-weight CYP2D6 EM patients, and may not reach therapeutic concentrations at all in the first 28 days of brexpiprazole administration. Obese CYP2D6 PM patients treated according to the disclosed regimens have similar Cₘᵢₙ during treatment initiation and reach therapeutic concentrations that are similar to normal-weight CYP2D6 EM patients that are treated according to the brexpiprazole FDA label.

FIG. 5A shows the mean AUC₂₄ of brexpiprazole in normal-weight CYP2D6 EM patients with MDD that are treated according to the brexpiprazole FDA label, with a starting dose of 0.5 mg (“Expected AUC₂₄”), obese CYP2D6 EM treated according to the brexpiprazole FDA label, with a starting dose of 0.5 mg, and obese CYP2D6 EM patients treated according to the disclosure (e.g., Table G, Modified Dosing Regimen A.1 and B.1). Obese CYP2D6 EM patients treated according to the brexpiprazole FDA label take longer to reach therapeutic concentrations of brexpiprazole than normal-weight CYP2D6 EM patients, and may not reach therapeutic concentrations at all in the first 28 days of brexpiprazole administration. Obese CYP2D6 EM patients treated according to the disclosed regimens have similar AUC₂₄ during treatment initiation and reach therapeutic concentrations that are similar to normal-weight CYP2D6 EM patients that are treated according to the brexpiprazole FDA label.

FIG. 5B shows the Cₘₐₓ of brexpiprazole in normal-weight CYP2D6 EM patients with MDD that are treated according to the brexpiprazole FDA label, with a starting dose of 0.5 mg (“Expected Cₘₐₓ), obese CYP2D6 EM treated according to the brexpiprazole FDA label, with a starting dose of 0.5 mg, and obese CYP2D6 EM patients treated according to the disclosure (e.g., Table G, Modified Dosing Regimen A.1 and B.1). Obese CYP2D6 EM patients treated according to the brexpiprazole FDA label take longer to reach therapeutic concentrations of brexpiprazole than normal-weight CYP2D6 EM patients, and may not reach therapeutic concentrations at all in the first 28 days of brexpiprazole administration. Obese CYP2D6 EM patients treated according to the disclosed regimens have similar Cₘₐₓ during treatment initiation and reach therapeutic concentrations that are similar to normal-weight CYP2D6 EM patients that are treated according to the brexpiprazole FDA label.

FIG. 5C shows the Cₘᵢₙ of brexpiprazole in normal-weight CYP2D6 EM patients with MDD that are treated according to the brexpiprazole FDA label, with a starting dose of 0.5 mg (“Expected Cₘᵢₙ), obese CYP2D6 EM treated according to the brexpiprazole FDA label, with a starting dose of 0.5 mg, and obese CYP2D6 EM patients treated according to the disclosure (e.g., Table G, Modified Dosing Regimen A.1 and B.1). Obese CYP2D6 EM patients treated according to the brexpiprazole FDA label take longer to reach therapeutic concentrations of brexpiprazole than normal-weight CYP2D6 EM patients, and may not reach therapeutic concentrations at all in the first 28 days of brexpiprazole administration. Obese CYP2D6 EM patients treated according to the disclosed regimens have similar Cₘᵢₙ during treatment initiation and reach therapeutic concentrations that are similar to normal-weight CYP2D6 EM patients that are treated according to the brexpiprazole FDA label.

FIG. 6A shows the AUC₂₄ of brexpiprazole in normal-weight CYP2D6 EM patients with MDD treated according to the brexpiprazole FDA label, with a starting dose of 0.5 mg (“Expected AUC₂₄”), normal-weight CYP2D6 PM patients with MDD that are treated according to the brexpiprazole FDA label, with a starting dose 0.25 mg, and normal-weight CYP2D6 PM patients treated according to the disclosure (e.g., Table G Modified Dosing Regimen Q.1). Normal-weight CYP2D6 PM patients take longer to reach therapeutic concentrations of brexpiprazole than normal-weight CYP2D6 EM patients, and may not reach therapeutic concentrations at all in the first 28 days of brexpiprazole administration. Normal-weight CYP2D6 PM patients treated according to the disclosed regimens have similar AUC₂₄ during treatment initiation and reach therapeutic concentrations that are similar to normal-weight CYP2D6 EM patients that are treated according to the brexpiprazole FDA label.

FIG. 6B shows the Cₘₐₓ of brexpiprazole in normal-weight CYP2D6 EM patients with MDD treated according to the brexpiprazole FDA label, with a starting dose of 0.5 mg (“Expected Cₘₐₓ”), normal-weight CYP2D6 PM patients with MDD that are treated according to the brexpiprazole FDA label, with a starting dose 0.25 mg, and normal-weight CYP2D6 PM patients treated according to the disclosure (e.g., Table G, Modified Dosing Regimen Q.1). Normal-weight CYP2D6 PM patients treated according FDA brexpiprazole label take longer to reach therapeutic concentrations of brexpiprazole than normal-weight CYP2D6 EM patients, and may not reach therapeutic concentrations at all in the first 28 days of brexpiprazole administration. Normal-weight CYP2D6 PM patients treated according to the disclosed regimens have similar Cₘₐₓ during treatment initiation and reach therapeutic concentrations that are similar to normal-weight CYP2D6 EM patients that are treated according to the brexpiprazole FDA label.

FIG. 6C shows the Cₘᵢₙ of brexpiprazole in normal-weight CYP2D6 EM patients with MDD treated according to the brexpiprazole FDA label, with a starting dose of 0.5 mg (“Expected Cₘᵢₙ”), normal-weight CYP2D6 PM patients with MDD that are treated according to the brexpiprazole FDA label, with a starting dose 0.25 mg, and normal-weight CYP2D6 PM patients treated according to the disclosure (e.g., Table G, Modified Dosing Regimen Q.1). Normal-weight CYP2D6 PM patients take longer to reach therapeutic concentrations of brexpiprazole than normal-weight CYP2D6 EM patients, and may not reach therapeutic concentrations at all in the first 28 days of brexpiprazole administration. Normal-weight CYP2D6 PM patients treated according to the disclosed regimens have similar Cₘᵢₙ during treatment initiation and reach therapeutic concentrations that are similar to normal-weight CYP2D6 EM patients that are treated according to the brexpiprazole FDA label.

FIG. 7A shows the mean AUC₂₄ of brexpiprazole in normal-weight CYP2D6 EM patients with MDD treated according to the brexpiprazole FDA label, with a starting dose of 1 mg (“Expected AUC₂₄”), obese CYP2D6 PM patients with MDD that are treated according to the brexpiprazole FDA label, with a starting dose of 0.5 mg, and obese CYP2D6 PM patients treated according to the disclosure (e.g., Table G, Modified Dosing Regimen G.1 and H.1). Obese CYP2D6 PM patients treated according to the brexpiprazole FDA label take longer to reach therapeutic concentrations of brexpiprazole than normal-weight CYP2D6 EM patients, and may not reach therapeutic concentrations at all in the first 28 days of brexpiprazole administration. Obese CYP2D6 PM patients treated according to the disclosed regimens have similar AUC₂₄ during treatment initiation and reach therapeutic concentrations that are similar to normal-weight CYP2D6 EM patients that are treated according to the brexpiprazole FDA label.

FIG. 7B shows the Cₘₐₓ of brexpiprazole in normal-weight CYP2D6 EM patients with MDD treated according to the brexpiprazole FDA label, with a starting dose of 1 mg (“Expected Cₘₐₓ”), obese CYP2D6 PM patients with MDD that are treated according to the brexpiprazole FDA label, with a starting dose of 0.5 mg, and obese CYP2D6 PM patients treated according to the disclosure (e.g., Table G, Modified Dosing Regimen G.1 and H.1). Obese CYP2D6 PM patients treated according to the brexpiprazole FDA label take longer to reach therapeutic concentrations of brexpiprazole than normal-weight CYP2D6 EM patients, and may not reach therapeutic concentrations at all in the first 28 days of brexpiprazole administration. Obese CYP2D6 PM patients treated according to the disclosed regimens have similar Cₘₐₓ during treatment initiation and reach therapeutic concentrations that are similar to normal-weight CYP2D6 EM patients that are treated according to the brexpiprazole FDA label.

FIG. 7C shows the Cₘᵢₙ of brexpiprazole in normal-weight CYP2D6 EM patients with MDD treated according to the brexpiprazole FDA label, with a starting dose of 1 mg (“Expected Cₘᵢₙ”), obese CYP2D6 PM patients with MDD that are treated according to the brexpiprazole FDA label, with a starting dose of 0.5 mg, and obese CYP2D6 PM patients treated according to the disclosure (e.g., Table G, Modified Dosing Regimen G.1 and H.1). Obese CYP2D6 PM patients treated according to the brexpiprazole FDA label take longer to reach therapeutic concentrations of brexpiprazole than normal-weight CYP2D6 EM patients, and may not reach therapeutic concentrations at all in the first 28 days of brexpiprazole administration. Obese CYP2D6 PM patients treated according to the disclosed regimens have similar Cₘᵢₙ during treatment initiation and reach therapeutic concentrations that are similar to normal-weight CYP2D6 EM patients that are treated according to the brexpiprazole FDA label.

FIG. 8A shows the mean AUC₂₄ of brexpiprazole in normal-weight CYP2D6 (“Expected AUC₂₄”) and obese CYP2D6 EM patients with MDD that are treated according to the brexpiprazole FDA label, with a starting dose of 1 mg, and obese CYP2D6 EM patients treated according to the disclosure (e.g., Table G, Modified Dosing Regimen C.1 and D.1). Obese CYP2D6 EM patients treated according to the brexpiprazole FDA label take longer to reach therapeutic concentrations of brexpiprazole than normal-weight CYP2D6 EM patients, and may not reach therapeutic concentrations at all in the first 28 days of brexpiprazole administration. Obese CYP2D6 EM patients treated according to the disclosed regimens have similar AUC₂₄ during treatment initiation and reach therapeutic concentrations that are similar to normal-weight CYP2D6 EM patients that are treated according to the brexpiprazole FDA label.

FIG. 8B shows the Cₘₐₓ of brexpiprazole in normal-weight CYP2D6 EM patients (“Expected Cₘₐₓ”) and obese CYP2D6 EM patients with MDD that are treated according to the brexpiprazole FDA label, with a starting dose of 1 mg, and obese CYP2D6 EM patients treated according to the disclosure (e.g., Table G, Modified Dosing Regimen C.1 and D.1). Obese CYP2D6 EM patients treated according to the brexpiprazole FDA label take longer to reach therapeutic concentrations of brexpiprazole than normal-weight CYP2D6 EM patients, and may not reach therapeutic concentrations at all in the first 28 days of brexpiprazole administration. Obese CYP2D6 EM patients treated according to the disclosed regimens have similar Cₘₐₓ during treatment initiation and reach therapeutic concentrations that are similar to normal-weight CYP2D6 EM patients that are treated according to the brexpiprazole FDA label.

FIG. 8C shows the Cₘᵢₙ of brexpiprazole in normal-weight CYP2D6 EM (“Expected Cₘᵢₙ”) and obese CYP2D6 EM patients with MDD that are treated according to the brexpiprazole FDA label, with a starting dose of 1 mg, and obese CYP2D6 EM patients treated according to the disclosure (e.g., Table G, Modified Dosing Regimen C.1 and D.1). Obese CYP2D6 EM patients treated according to the brexpiprazole FDA label take longer to reach therapeutic concentrations of brexpiprazole than normal-weight CYP2D6 EM patients, and may not reach therapeutic concentrations at all in the first 28 days of brexpiprazole administration. Obese CYP2D6 EM patients treated according to the disclosed regimens have similar Cₘᵢₙ during treatment initiation and reach therapeutic concentrations that are similar to normal-weight CYP2D6 EM patients that are treated according to the brexpiprazole FDA label.

FIG. 9A shows the AUC₂₄ of brexpiprazole in normal-weight CYP2D6 EM (“Expected AUC₂₄”) and normal-weight CYP2D6 PM patients with MDD that are treated according to the brexpiprazole FDA label, with a starting dose of 1 mg and 0.5 mg, respectively, and normal-weight CYP2D6 EM patients treated according to the disclosure (e.g., Table G, Modified Dosing Regimen S.1). Normal-weight CYP2D6 PM patients take longer to reach therapeutic concentrations of brexpiprazole than normal-weight CYP2D6 EM patients, and may not reach therapeutic concentrations at all in the first 28 days of brexpiprazole administration. Normal-weight CYP2D6 PM patients treated according to the disclosed regimens have similar AUC₂₄ during treatment initiation and reach therapeutic concentrations that are similar to normal-weight CYP2D6 EM patients that are treated according to the brexpiprazole FDA label.

FIG. 9B shows the Cₘₐₓ of brexpiprazole in normal-weight CYP2D6 EM (“Expected Cₘᵢₙ”) and normal-weight CYP2D6 PM patients with MDD that are treated according to the brexpiprazole FDA label, with a starting dose of 1 mg and 0.5 mg, respectively, and normal-weight CYP2D6 PM patients treated according to the disclosure (e.g., Table G, Modified Dosing Regimen S.1). Normal-weight CYP2D6 PM patients treated according FDA brexpiprazole label take longer to reach therapeutic concentrations of brexpiprazole than normal-weight CYP2D6 EM patients, and may not reach therapeutic concentrations at all in the first 28 days of brexpiprazole administration. Normal-weight CYP2D6 PM patients treated according to the disclosed regimens have similar AUC₂₄ during treatment initiation and reach therapeutic concentrations that are similar to normal-weight CYP2D6 EM patients that are treated according to the brexpiprazole FDA label.

FIG. 9C shows the Cₘᵢₙ of brexpiprazole in normal-weight CYP2D6 EM (“Expected Cₘᵢₙ”) and normal-weight CYP2D6 PM patients with MDD that are treated according to the brexpiprazole FDA label, with a starting dose of 1 mg and 0.5 mg, respectively, and obese CYP2D6 PM patients treated according to the disclosure (e.g., Table G, Modified Dosing Regimen S.1). Normal-weight CYP2D6 PM patients take longer to reach therapeutic concentrations of brexpiprazole than normal-weight CYP2D6 EM patients, and may not reach therapeutic concentrations at all in the first 28 days of brexpiprazole administration.

### DETAILED DESCRIPTION

U.S. Pat. No. 11,229,644 is incorporated by reference in its entirety for all purposes. Embodiments of the present disclosure describing percent or milligram increases of the usual dose during treatment initiation can be combined with the U.S. Pat. No. 11,229,644 to achieve the total daily doses administering during treatment initiation disclosed in U.S. Pat. No. 11,229,644. In embodiments, the milligram or percentage increases of the usual dose during treatment initiation for an obese CYP2D6 EM patient (and half of the usual dose, when the obese patient is a CYP2D6 PM) may be administered once daily as described herein or twice daily as described in U.S. Pat. No. 11,229,644, respectively.

### Definitions

Any reference to brexpiprazole herein also encompasses all of the pharmaceutically acceptable isomers (e.g., stereoisomers), solvates, hydrates, polymorphs, salts, and prodrugs (e.g., esters and phosphates).

As used herein, the term “about” refers to an amount somewhat more or less than the stated parameter value, for example plus or minus five or ten percent of the object that “about” modifies, or as one of skill in the art would recognize from the context (e.g., approximately 50% of the interval between values). The term “about” also includes the value referenced. For example, a BMI of about 40 includes 40, as well as values somewhat below or above 40.

As used herein, “normal,” “normal-weight,” or other derivations or variations thereof refers to a non-obese state in a person who can have at least one of the following characteristics: BMI less than about 35 kg/m 2, % IBW less than about 150%, waist size less than about 42, % body fat less than about 40%, % android body fat less than about 40%, % gynoid body fat less than about 40%, and total body fat less than about 40 kg. Unless otherwise modified, “normal metabolizer” also means an extensive CYP2D6 metabolizer.

As used herein, the terms “reference dose”, “reference daily dose”, or “recommended dose”, refer to the maintenance dosage of brexpiprazole, as indicated on the manufacture's FDA-approved label (e.g., the most recent FDA-approved label in effect as of December 2021). The REXULTI® label also refers to a “Starting Dose” and a “Maximum Dose” as distinct from the “Recommended Dose”. While colloquially the term “recommended” dose could refer to any dose taught in the REXULTI® label, in this disclosure the term “recommended dose” refers more narrowly to doses recommended for maintenance treatment (including the “maximum dose” suitable for such treatment) of a normal weight, extensive CYP2D6 metabolizer patients. Thus, where the REXULTI® label teaches administering 2 mg once daily up to a maximum dose of 3 mg once daily for an MDD patient, such dose or dose range is a “recommended” dose or dose range. Where the REXULTI® label teaches reducing the “usual dosage by half” for “Known CYP2D6 Poor Metabolizers”, the recommended dose as used herein is the dose “recommended” for patients who are not CYP2D6 PMs.

It is common for a particular drug to be approved for multiple different indications, and each indication may have a different reference or recommended dose. For example, the “recommended dose” listed in the December 2021 REXULTI® label indicates that 2 to 3 mg once daily is the “recommended dose” for MDD, and 2 to 4 mg once daily is the “recommended dose” for schizophrenia.

As used herein, “usual dose” refers to the dose that a patient who is not a CYP2D6 PM (i.e., a patient that is a CYP2D6 extensive metabolizer) would receive on the same day of treatment according to the December 2021 REXULTI® label. For example, the usual dose for treating schizophrenia in an adult patient on Day 1 of treatment (i.e., the starting dose) is 1 mg/day, and the usual dose for treating schizophrenia in a pediatric patient ages 13-17 on Day 1 (i.e., the starting dose) is 0.5 mg. On Day 5 of treatment, the usual dose for treating schizophrenia in an adult patient is 2 mg/day, and the usual dose for treating schizophrenia in a pediatric patient ages 13-17 is 1 mg/day. Where the REXULTI® label teaches reducing the “usual dosage by half” for “Known CYP2D6 Poor Metabolizers”, the usual dose as used herein is the dose “usual” for patients who are not CYP2D6 PMs. Thus, the usual dose for treating schizophrenia in an adult patient on Day 1 (i.e., the starting dose) is 1 mg/day, and the REXULTI® label instructs CYP2D6 PM patients to take half of the usual dose, which is 0.5 mg.

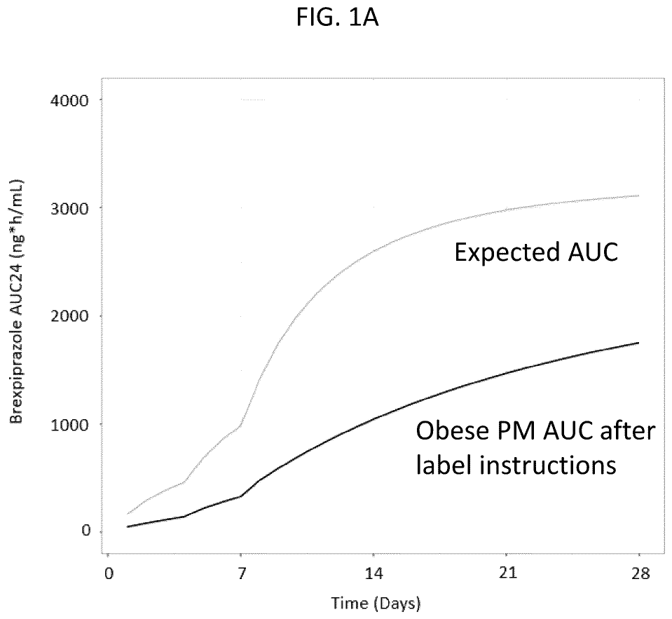

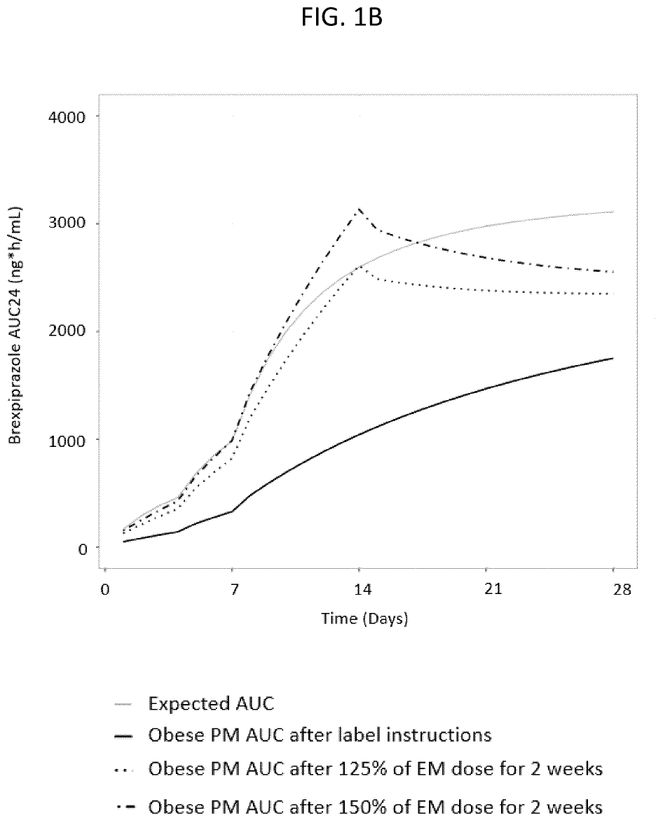

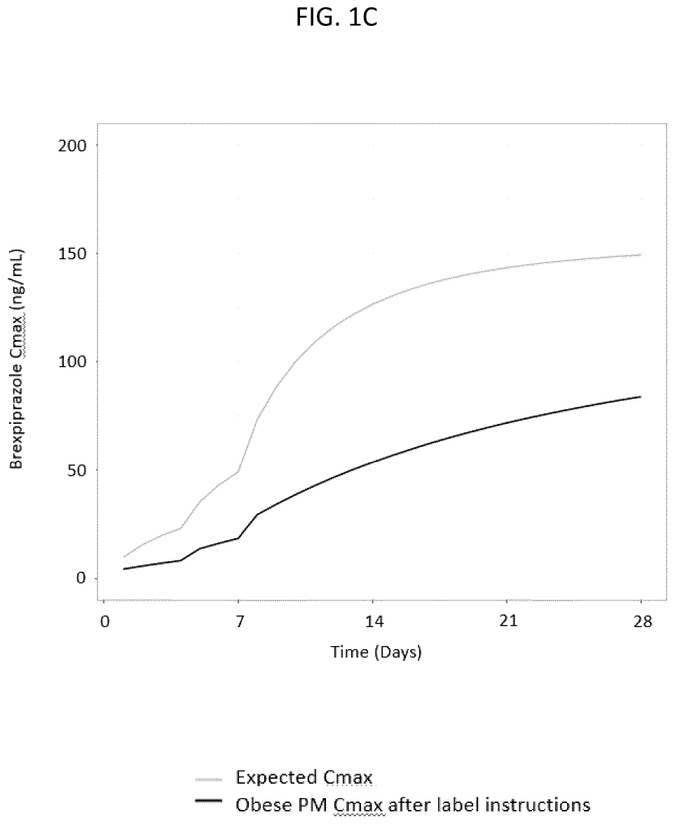

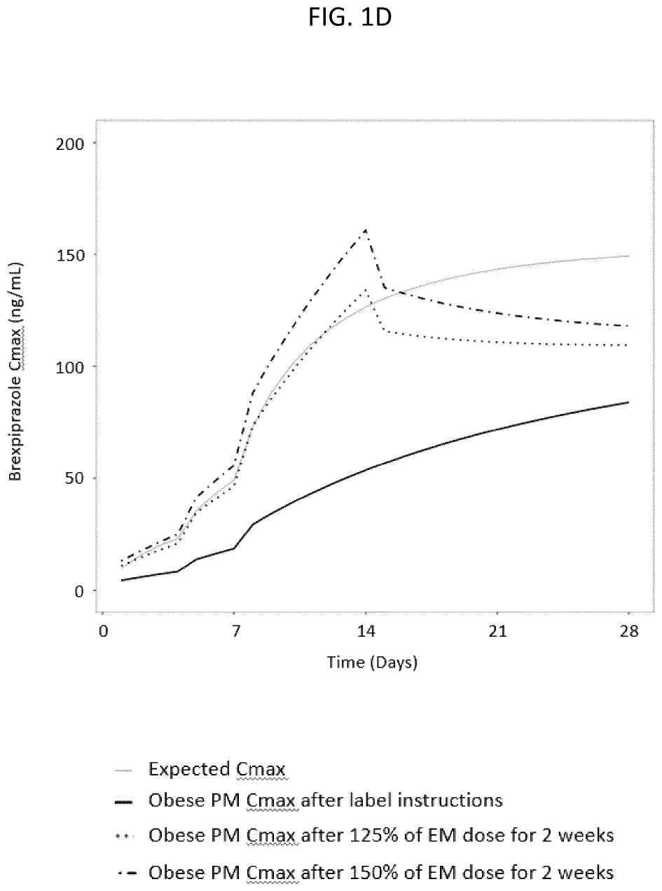

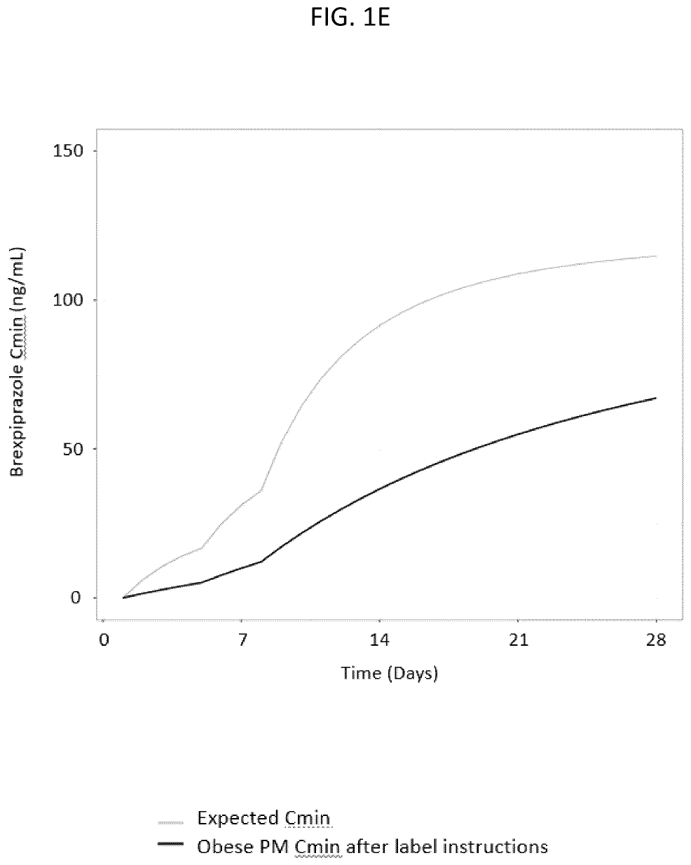

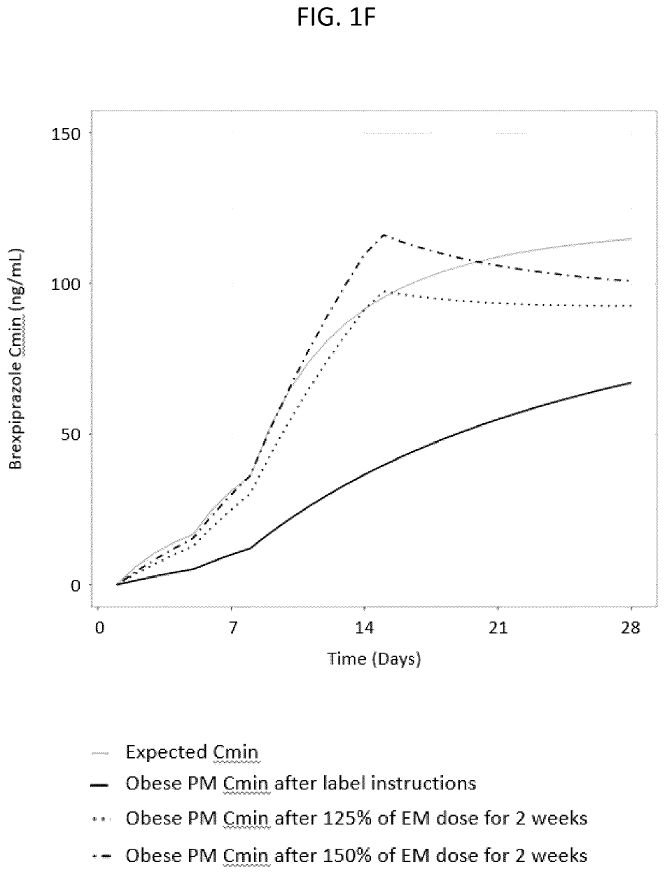

As used herein “therapeutic concentration” refers to the steady state pharmacokinetic profile of brexpiprazole based on the pharmacokinetic studies supporting FDA approval of brexpiprazole as measured in normal-weight patients. As shown in FIG. 1A and FIG. 1B, normal-weight patients treated with brexpiprazole achieve steady state pharmacokinetics after the initiation phase of treatment, typically around days 14-21 of treatment according to FIG. 1A and FIG. 1B. Because the blood plasma levels in FIG. 1A and FIG. 1B represent averages from all patients, some deviation from the average steady state pharmacokinetic profile in a particular patient or patient population is expected and is acceptable in the art. The modified dosing regimens of the present disclosure bring the blood plasma concentrations of brexpiprazole in obese patients and obese CYP2D6 PM patients within appropriate degrees of variation of the average steady state pharmacokinetic profile of normal-weight CYP2D6 EM patients shown in FIG. 1A and FIG. 1B. This enables obese CYP2D6 EM patients or obese CYP2D6 PM patients to reach therapeutic concentrations of brexpiprazole at a similar time as normal-weight CYP2D6 EM patients, allowing for better clinical response. It is not necessary for the pharmacokinetic profile of the modified dosing regimens disclosed herein to overlap exactly with the pharmacokinetic profile of brexpiprazole based on the pharmacokinetic studies supporting FDA approval.

As used herein “QD” refers to once daily administration.

As used herein, “initiation” refers to the time period during when the patient is first treated with brexpiprazole up to the day when the patient reaches steady state blood plasma concentrations of brexpiprazole. Treatment initiation lasts several days. In embodiments, the “initiation” period may last from 7-21 days from Day 1 of treatment depending on the starting dose, indication, and CYP2D6 status. During the “initiation” phase of treatment, a patient generally does not reach therapeutically effective blood plasma levels of brexpiprazole. Therapeutically effective blood plasma levels are generally achieved after the initiation period when a patient receives the recommended and maximum doses identified in the drug label.

A number of days (e.g., Day 1, Days 8-14, etc.) is used herein to refer to the day of brexpiprazole treatment. For example, “Day 1” refers to the first day of brexpiprazole treatment. Accordingly, Day 1 AUC₂₄ refers to the AUC₂₄ measured on the first day of being treated with brexpiprazole. As another example, “the first 7 days” refers to Days 1, 2, 3, 4, 5, 6, and 7 of brexpiprazole treatment.

As used herein, “pediatric” refers to a patient under the age of 18. In embodiments, the pediatric patient is 6-17 years old. In embodiments, the pediatric patient is 13-17 years old. In embodiments, the pediatric patient is 6-13 years old. In embodiments, the pediatric patient is 6 years old up to but not including 13.

Brexpiprazole

Brexpiprazole is an atypical antipsychotic, available as REXULTI®, to be used as an adjunctive therapy to antidepressants for the treatment of major depressive disorder and as a treatment for schizophrenia. The FDA label of REXULTI® (Otsuka and Lundbeck, revised December 2021) is incorporated by reference herein in its entirety. Brexpiprazole is 7-14-14-(1-Benzothiophen-4-yl)piperazin-1-yllbutoxylquinolin-2(1H)-one. The empirical formula is C₂₅H₂₇N₃O₂S and its molecular weight is 433.57.

The chemical structure of brexpiprazole is

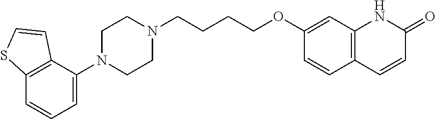 [SMILES: O=c1ccc2ccc(OCCCCN3CCN(c4cccc5sccc45)CC3)cc2[nH]1]

The brexpiprazole dosage regimen recommended by the FDA label (REXULTI®) is shown in Table 1.

Brexpiprazole treatment is initiated in a conservative manner, as elevating the dose too quickly exposes the patient to the risk of significant, unpleasant side effects such as akathisia, as well as other serious adverse reactions described in the FDA label. When patients experience such side effects they are often reluctant to continue treatment. Because the risks of untreated depression or schizophrenia are so significant, it is important to initiate treatment in a manner that minimizes the likelihood of such side effects. This is why the “Dosage and Administration” section of the Rexulti label provides for a low, initial dose of brexpiprazole, which is then elevated at specified intervals (depending on the indication treated) up to the target (i.e., maintenance or maximum) dose. This gradual elevation of the brexpiprazole dose allows the patient's physiology time to adapt to the effects of brexpiprazole before escalating to the higher doses needed to achieve the desired therapeutic effects. This gradual elevation of the brexpiprazole dose minimizes the likelihood that the patient will experience side effects, and thus decreases the risk of patient non-adherence or outright treatment refusal.

For schizophrenia in adults, the starting dosage for brexpiprazole is 1 mg once daily on Days 1 to 4, taken orally with or without food. The recommended brexpiprazole dosage is 2 mg to 4 mg once daily. Titrate to 2 mg once daily on Day 5 through Day 7, then to 4 mg on Day 8 based on the patient's clinical response and tolerability. Dose adjustments between the “Recommended Dose” and “Maximum Dose” are based on the patient's clinical response and tolerability.

For schizophrenia in pediatrics (13-17), the starting dosage for brexpiprazole is 0.5 mg once daily on Days 1 to 4, taken orally with or without food. Patients titrate to 1 mg once daily on Day 5 through 7, then to 2 mg on Day 8. Thereafter, weekly dose increases can be made in 1 mg increments. Dose increases are based on the patient's clinical response and tolerability. The recommended dosage is 2 mg to 4 mg once daily, and the maximum dose is 4 mg. Dose adjustments between the “Recommended Dose” and “Maximum Dose” are based on the patient's clinical response and tolerability.

For major depressive disorder, the starting dosage for brexpiprazole as adjunctive treatment is 0.5 mg or 1 mg once daily, taken orally with or without food. Dose increases occur on weekly intervals. Patients titrate to 1 mg once daily, then up to the recommended dosage of 2 mg once daily. The maximum recommended daily dosage is 3 mg. Dose adjustments between the “Recommended Dose” and “Maximum Dose” are based on the patient's clinical response and tolerability.

TABLE 1
Brexpiprazole Dosing According to FDA Label

| | | | Recommended | | Maximum |
|---|---|---|---|---|---|
| Indication | Starting Dose | | Dose | | Dose |
| Schizophrenia Adults | 1 | mg/day | 2-4 | mg/day | 4 mg/day |
| Schizophrenia | 0.5 | mg/day | 2-4 | mg/day | 4 mg/day |
| Pediatrics (13-17) | | | | | |
| Major depressive | 0.5 or 1 | mg/day | 2 | mg/day | 3 mg/day |
| disorder (MDD) | | | | | |
| Adults | | | | | |

The FDA label provides for specific “starting doses” during initiation. While the label recommends dose adjustments based on the patient's clinical response and tolerability, such dose adjustments refer to adjustments between then “Recommended Dose” and “Maximum Dose,” since a patient does not exhibit a clinical response to treatment until receiving the “Recommended Dose” after completing the initiation phase of treatment.

The FDA label provides dosage modifications for CYP2D6 Poor Metabolizers and for concomitant use with CYP Inhibitors or Inducers: Dosage adjustments are recommended in patients who are known cytochrome P450 (CYP) 2D6 poor metabolizers and in patients taking concomitant CYP3A4 inhibitors or CYP2D6 inhibitors or strong CYP3A4 inducers (see Table 2). If the coadministered drug is discontinued, adjust the REXULTI dosage to its original level. If the coadministered CYP3A4 inducer is discontinued, reduce the REXULTI dosage to the original level over 1 to 2 weeks.

TABLE 2
Brexpiprazole Dosing for Certain Patient Subpopulations

| | |
|---|---|
| | Adjusted Brexpiprazole (REXULTI ®) |
| Factors | Dosage |

CYP2D6 Poor Metabolizers

| | |
|---|---|
| CYP2D6 poor metabolizers | Administer half of the usual dose. |
| Known CYP2D6 poor metabolizers taking | Administer a quarter of the usual dose. |
| strong/moderate CYP3A4 inhibitors | |

Patients taking CYP2D6 Inhibitors and/or CYP3A4 Inhibitors

| | |
|---|---|
| Strong CYP2D6 inhibitors (paroxetine, | Administer half of the usual dose. |
| fluoxetine) | |
| Strong CYP3A4 inhibitors | Administer half of the usual dose. |
| Strong/moderate CYP2D6 inhibitors with | Administer a quarter of the usual dose. |
| strong/moderate CYP3A4 inhibitors | |

Patients taking CYP3A4 Inducers

| | |
|---|---|
| Strong CYP3A4 Inducers | Double usual dose over 1 to 2 weeks |

Applicant found that the brexpiprazole (REXULTI®) dosage instructions for initiating treatment with brexpiprazole do not provide therapeutically effective levels of brexpiprazole for patients that are obese or obese CYP2D6 PM as quickly as for normal-weight CYP2D6 EMs.

Expected Drug Profile

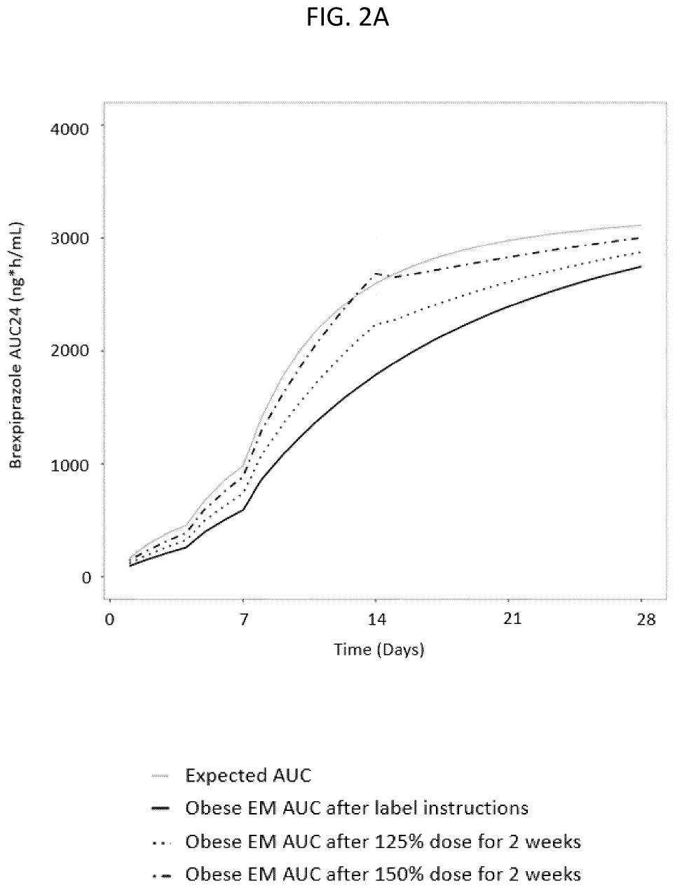

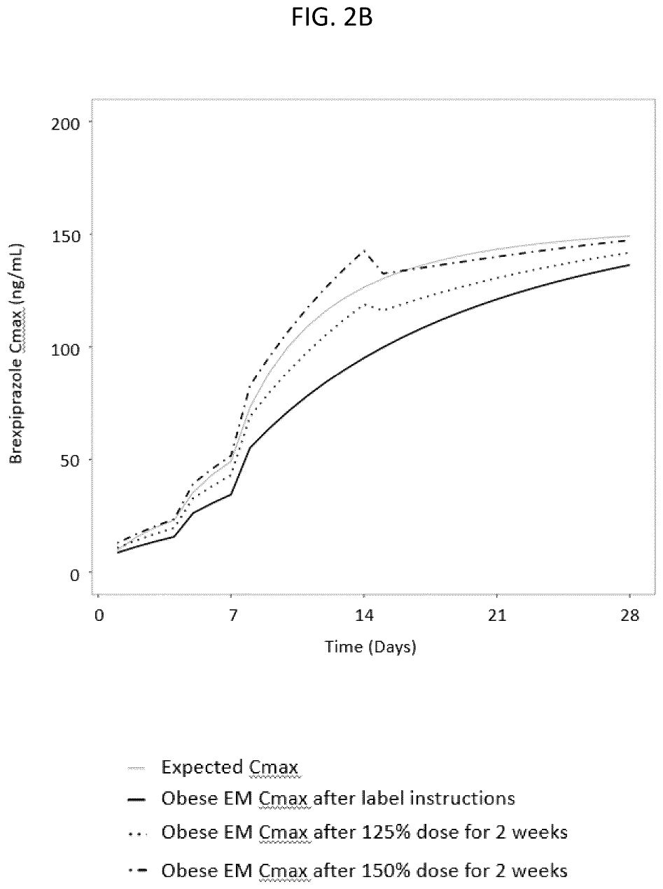

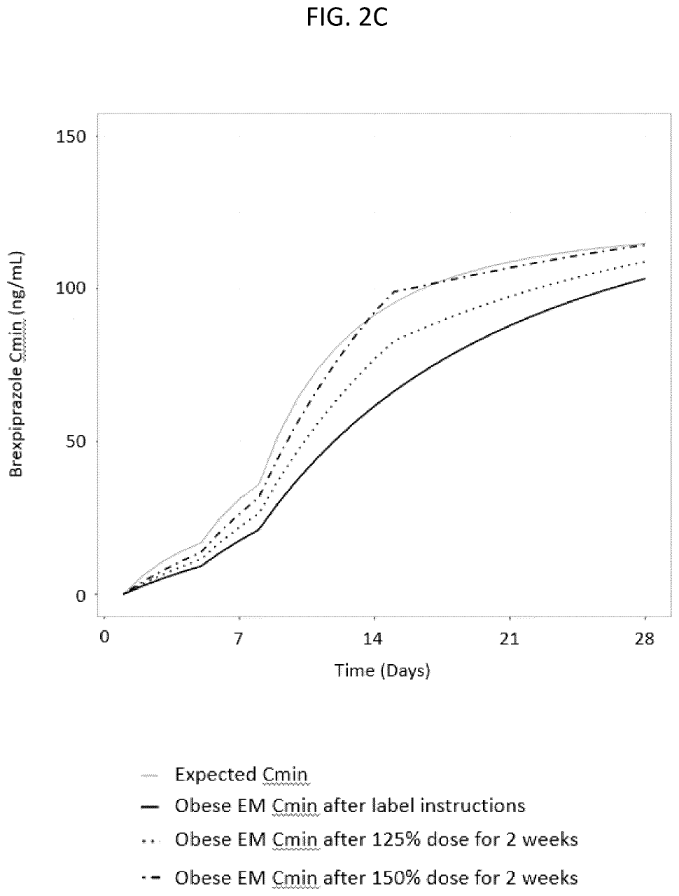

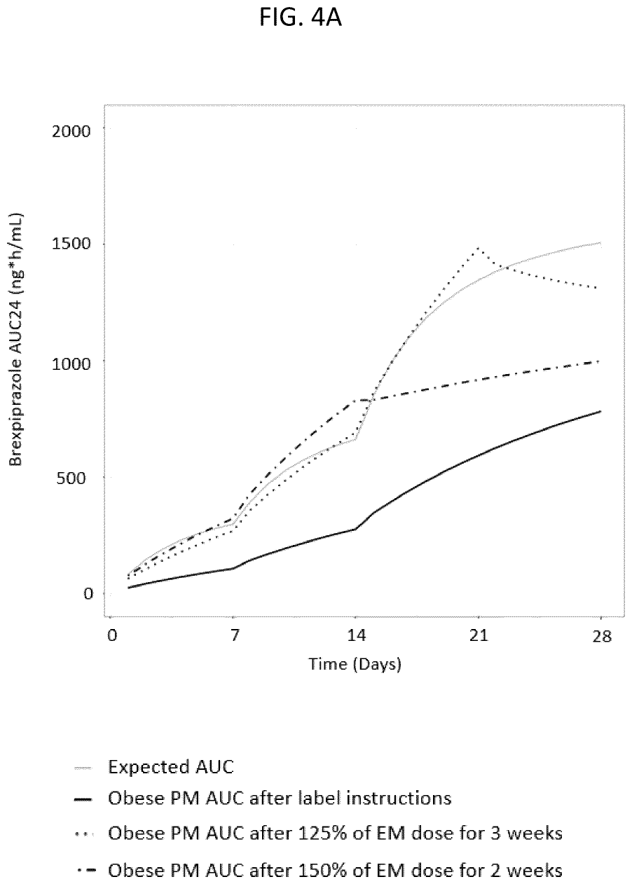

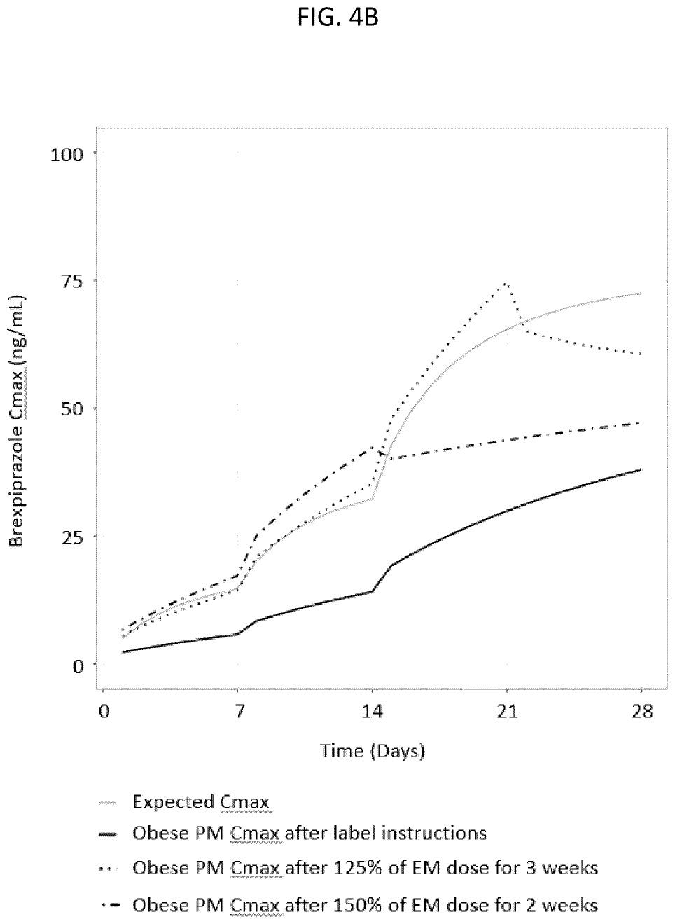

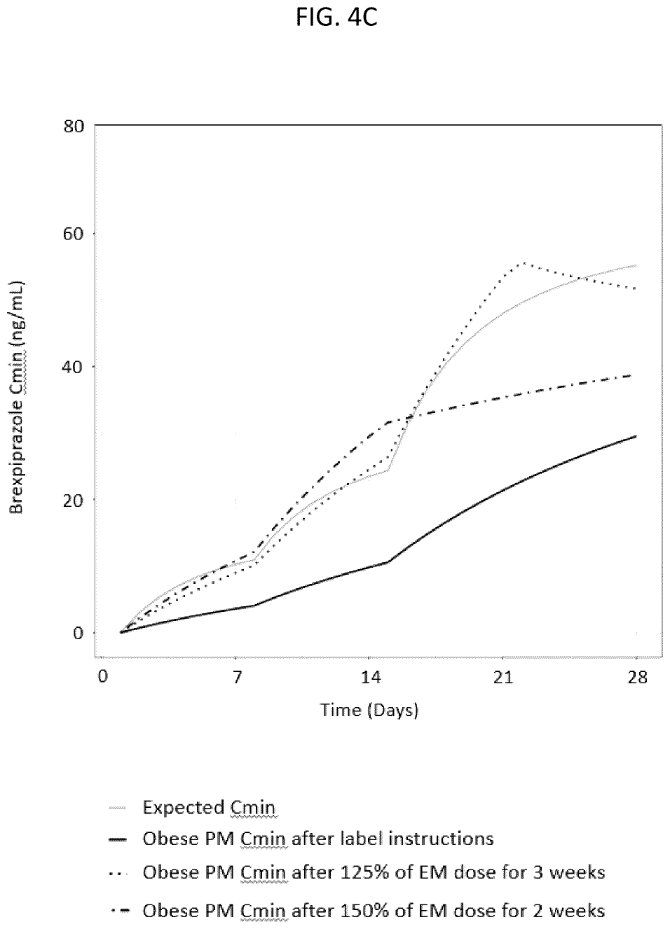

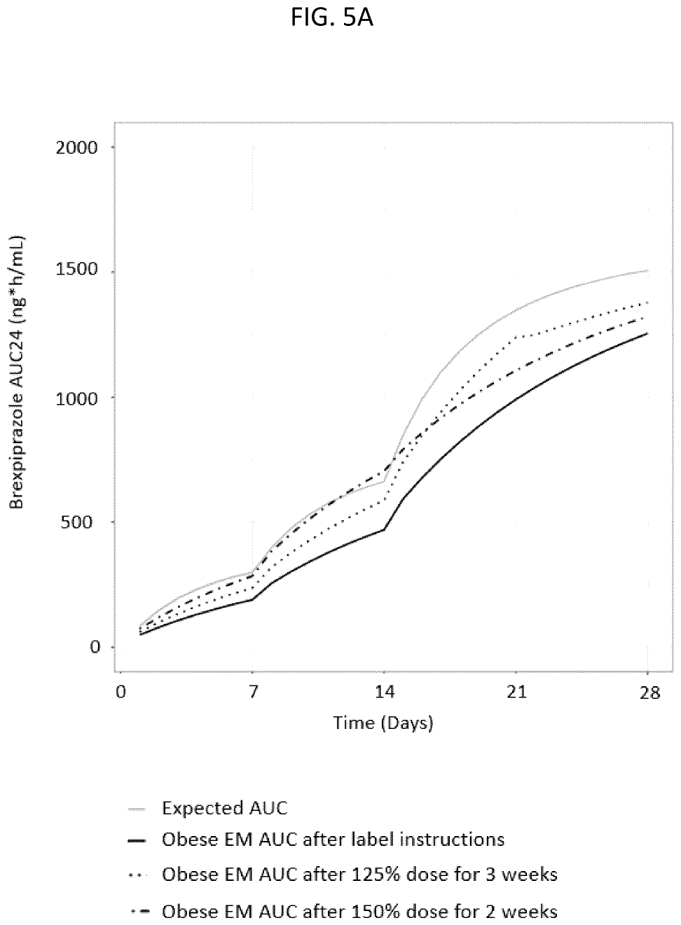

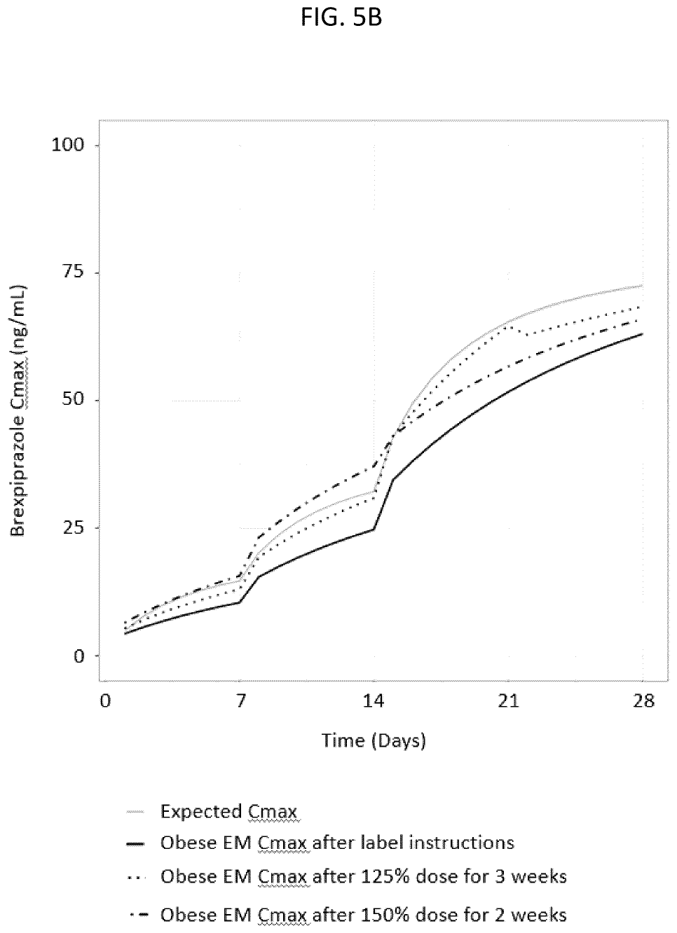

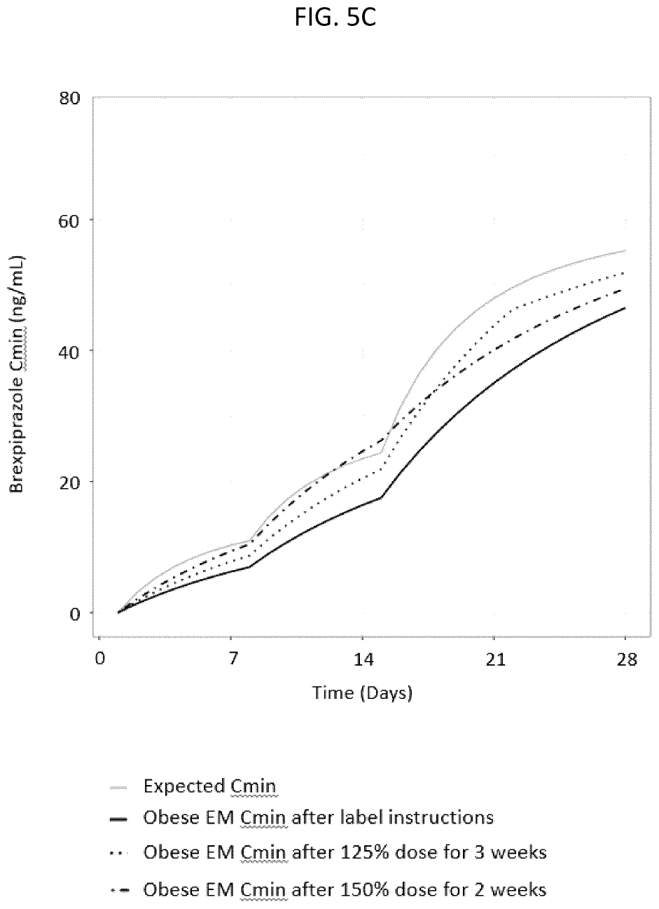

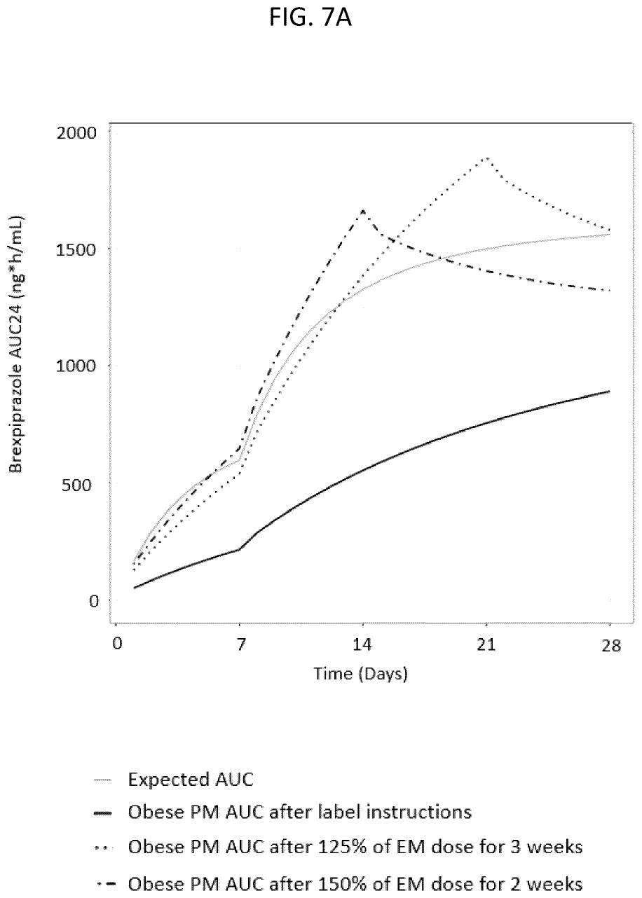

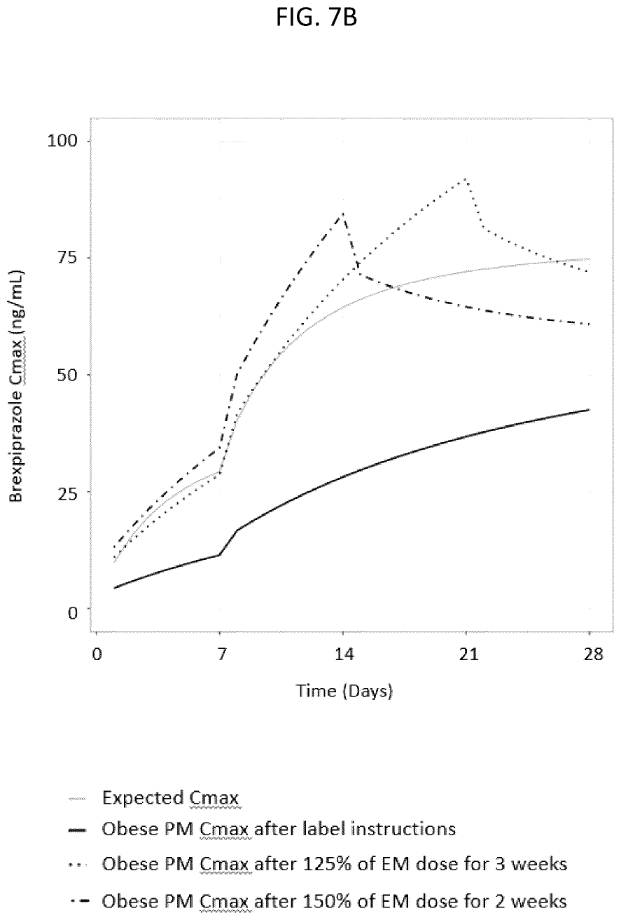

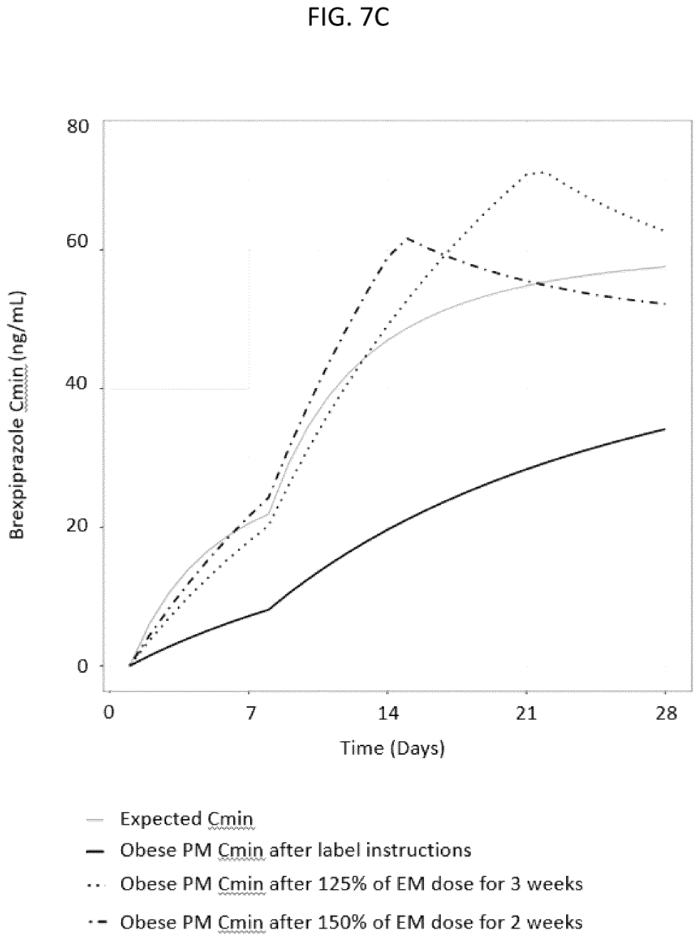

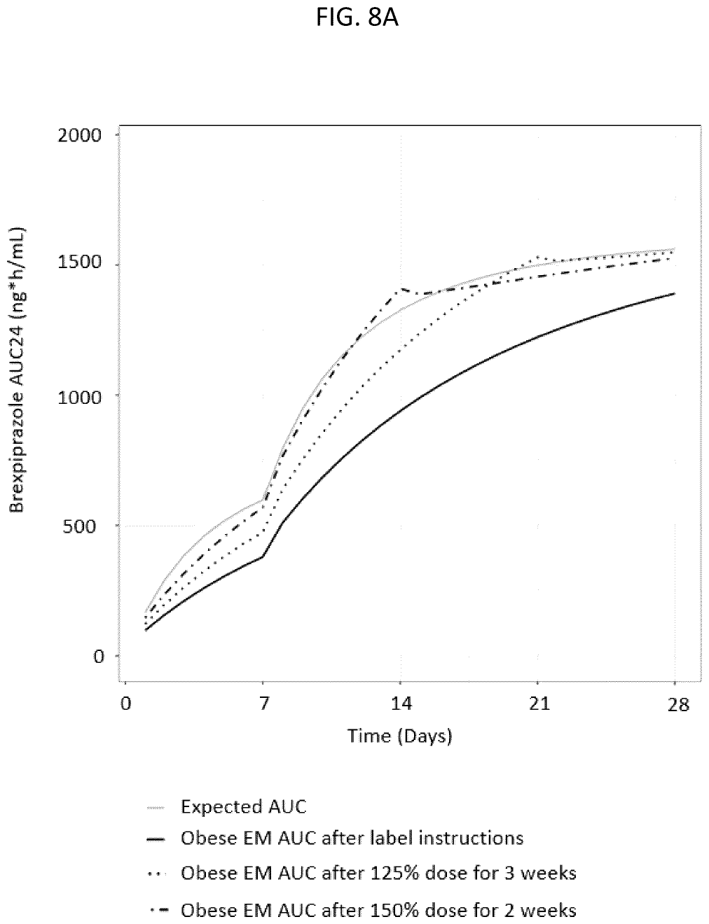

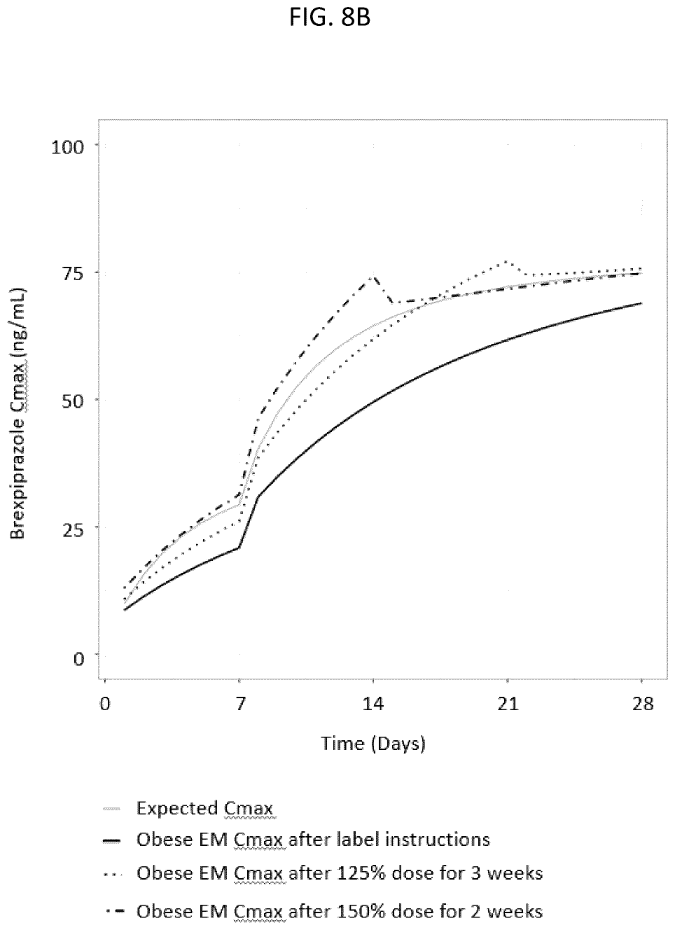

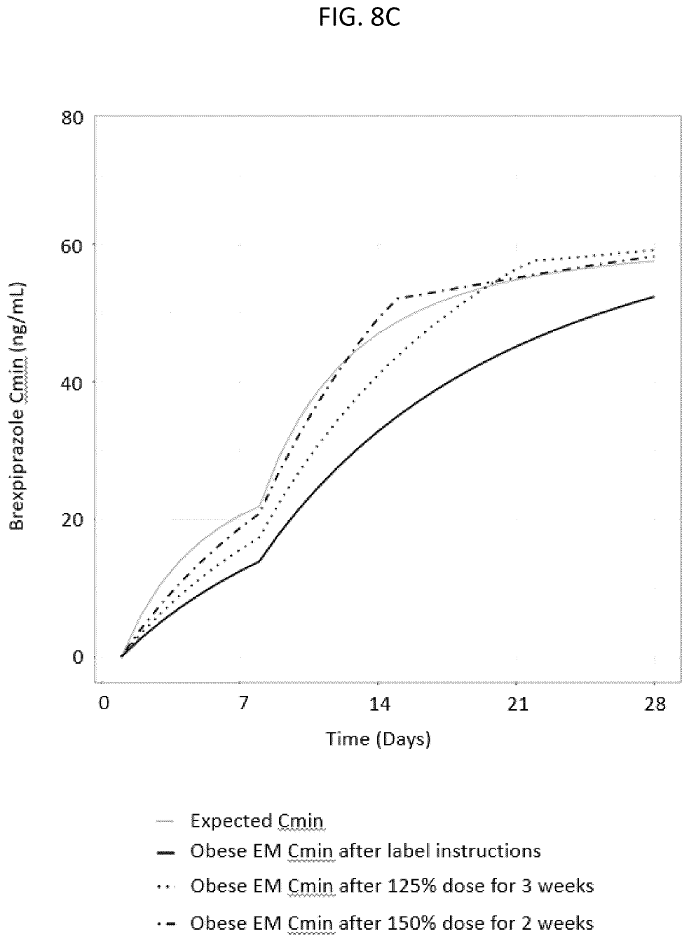

The expected blood plasma brexpiprazole concentrations (AUC, Cₘₐₓ, and Cₘᵢₙ) during the initiation of brexpiprazole treatment (days 1-14 or 1-28) for obese patients with schizophrenia and major depressive disorder (MDD) according to the brexpiprazole FDA label (revised in December 2021) are shown in FIG. 1A, FIG. 1C, FIG. 1E, FIG. 2A-2C, FIG. 4A-4C, FIG. 5A-5C, FIG. 7A-7C, and FIG. 8A-8C. Because there was no recognition in the art that the dosing regimen for initiating treatment with brexpiprazole should be adjusted based on the obesity status of the patient, obesity status was not expected to affect the blood plasma concentration of brexpiprazole. In other words, obese patients with schizophrenia or major depressive disorder were expected to have qualitatively similar blood plasma concentrations as normal weight patients.

The present inventors are not aware of anything in the art which would contradict the use of the same brexpiprazole dosing regimen for obese and normal-weight patients disclosed in the FDA-approved label for brexpiprazole. It is acknowledged in the art that body size and obesity can have an effect on the pharmacokinetics of some drugs; however, the clinical relevance of this effect is highly dependent on the particular characteristics of that drug. For example, Hanley et al., in reviewing the effects of obesity on drug pharmacokinetics, found that appropriate drug dosing should be individualized to the particular drug at issue, and that the distribution of a drug in obese patients cannot be entirely predicted based on the physiochemical attributes of the drug (e.g., lipophilicity, hydrophilicity) alone. (Hanley et al., Effect of Obesity on the Pharmacokinetics of Drugs in Humans, Clin. Pharmacokinet 2010, 49(2): 70-87.) The pharmacokinetic studies leading to the approval of brexpiprazole did not include patients with BMI>35 kg/m 2, and previous studies have found that the effect of weight on the pharmacokinetics of brexpiprazole was less than 20% and was not a significant determinant in brexpiprazole pharmacokinetics. The inventors are not aware of any evidence in the prior art that suggests that there is any clinically important effect of obesity on brexpiprazole pharmacokinetics that would require any difference in the brexpiprazole dosing regimen between obese and normal-weight patients. Thus, at the time of the present application, the FDA-approved dosing instructions for obese and normal weight patients are the same. However, the present invention is based on the discovery that a patient's body size significantly affects the time it takes a patient to reach therapeutic levels of brexpiprazole. See FIG. 1A, FIG. 1C, FIG. 1E, FIG. 2A-2C, FIG. 4A-4C, FIG. 5A-5C, FIG. 7A-7C, and FIG. 8A-8C.

Without this new information, it was not appreciated in the art that, using the instructions for brexpiprazole dosing found in the existing FDA-approved labels for brexpiprazole (at the time of the present disclosure), obese patients do not reach therapeutic levels of brexpiprazole as quickly as normal weight patients upon initiating brexpiprazole treatment; or alternatively stated, it has been discovered that it takes significantly longer to reach therapeutic levels of brexpiprazole in obese patients compared to normal-weight patients using the FDA-approved label's instructions.

The REXULTI® label teaches reducing the dose of brexpiprazole to half of the usual dosage if the patient is a CYP2D6 poor metabolizer (CYP2D6 PMs or simply “PMs”). Similarly, the present inventors have also found that the time required to reach therapeutic levels of brexpiprazole for obese patients who are also PMs, using the FDA-approved brexpiprazole dosing regimen (i.e., half of the recommended dose), is longer than for normal-weight PMs. See FIG. 1A, FIG. 1C, FIG. 1E, FIG. 4A-4C, and FIG. 7A-7C.

Thus, prior to the present invention, for obese patient populations (as described herein), the patient's psychiatric disorders (e.g., schizophrenia and major depressive disorder) were unknowingly left undertreated because these patients did not reach therapeutic concentrations in a similar time as normal-weight patient. Such unintended undertreatment of psychiatric disorders is potentially quite serious, as complications of untreated psychiatric disorders include suicide attempts, anxiety, depression, alcohol or drug abuse, inability to work or attend school, financial problems, homelessness, social isolation, health and medical problems, being victimized, and aggressive behavior.

Thus, the development of the presently disclosed new dosage regimens allow obese patients to reach therapeutic levels of brexpiprazole as quickly as normal-weight patients without putting them at increased risk of akathisia. See FIG. 1A, FIG. 1C, FIG. 1E, FIG. 2A-2C, FIG. 4A-4C, FIG. 5A-5C, FIG. 7A-7C, and FIG. 8A-8C.

The present disclosure provides an alternative dosing regimen for treating a patient with a psychiatric disorder, such as schizophrenia or major depressive disorder, with brexpiprazole, wherein the patient has one or more of the following characteristics: (i) a BMI of at least about 35; (ii) % IBW of at least about 150%; (iii) waist size greater than about 42 inches; (iv) % body fat greater than about 40%; (v) % android body fat greater than about 40%; (vi) % gynoid body fat greater than about 40%; (vii) total body fat greater than about 40 kg; or (viii) CYP2D6 poor metabolizer.

The FDA label for brexpiprazole neither recognizes that obese CYP2D6 EM patients and/or obese CYP2D6 PM patients do not reach therapeutic levels as quickly as normal-weight CYP2D6 EM patients, nor does it provide a dosage regimen that corrects this (hitherto unknown) problem. Instead, the label implicitly teaches that obese CYP2D6 EM should receive the same dose as normal-weight CYP2D6 EM, and explicitly teaches that all CYP2D6 PM patients (i.e., normal-weight and obese) should receive half of the dose that CYP2D6 EM patients receive. However, Applicants have discovered that administering the same dose to obese CYP2D6 EM that normal-weight CYP2D6 EM receive, and half of the dose to obese CYP2D6 PM (as taught by the FDA label) causes obese CYP2D6 EM and obese CYP2D6 PM patients to reach therapeutic brexpiprazole concentrations more slowly than normal-weight CYP2D6 EM patients. Applicants have developed a modified brexpiprazole dosage regimen for initiating treatment with brexpiprazole that allows obese CYP2D6 EM and obese CYP2D6 PM patients to reach therapeutically effective concentrations at a similar time compared to normal-weight CYP2D6 EM patients (FIGS. 1B, 1D, 1F, 2A-2C, 4A-C, 5A-5C, 7A-7C and 8A-8C).

In embodiments, the disclosed methods comprise initiating treatment in obese patients (EM and PM) by administering a dose of brexpiprazole that is increased compared to the usual dosage of brexpiprazole administered on the same day of treatment initiation according to the brexpiprazole (REXULTI®) FDA label. In embodiments, after treatment initiation, the methods of the disclosure return to the reference dose. In embodiments, the dose of brexpiprazole administering during treatment initiation is increased by at least about 10%, e.g., about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195%, or about 200%, about 205%, about 210%, about 215%, about 220%, about 225%, about 230%, about 235%, about 240%, about 245%, about 250%, about 255%, about 260%, about 265%, about 270%, about 275%, about 280%, about 285%, about 290%, about 295%, about 300%, about 305%, about 310%, about 315%, about 320%, about 325%, about 330%, about 335%, about 340%, about 345%, about 350%, about 355%, about 360%, about 365%, about 370%, about 375%, about 380%, about 385%, about 390%, about 395%, or about 400%, about 405%, about 410%, about 415%, about 420%, about 425%, about 430%, about 435%, about 440%, about 445%, about 450%, about 455%, about 460%, about 465%, about 470%, about 475%, about 480%, about 485%, about 490%, about 495%, about 500%, about 605%, about 610%, about 615%, about 620%, about 625%, about 630%, about 635%, about 640%, about 645%, about 650%, about 655%, about 660%, about 665%, about 670%, about 675%, about 680%, about 685%, about 690%, about 695%, or about 700%, about 705%, about 710%, about 715%, about 720%, about 725%, about 730%, about 735%, about 740%, about 745%, about 750%, about 755%, about 760%, about 765%, about 770%, about 775%, about 780%, about 785%, about 790%, about 795%, about 800%, or more, inclusive of all values and ranges therein, compared to the usual dosage administered on the same day of treatment initiation according to the brexpiprazole FDA label. In embodiments, the dose of brexpiprazole administering during treatment initiation to obese CYP2D6 EM and obese CYP2D6 PM is increased by about 25% (i.e., the dose administered during the initiation period according to the modified dosing regimen disclosed herein is 125% of the usual dosage administered on the same day of treatment initiation according to the brexpiprazole (REXULTI®) FDA label). In embodiments, the dose of brexpiprazole administering during treatment initiation is increased by about 50% (i.e., the dose administered during the initiation period according to the modified dosing regimen disclosed herein is 125% of the usual dosage administered on the same day of treatment initiation according to the brexpiprazole (REXULTI®) FDA label). Surprisingly, the modified regimens of the disclosure administer similar increased doses (compared to the usual dose) to both obese CYP2D6 PM and obese CYP2D6 EM patients, even though the FDA label for brexpiprazole instructs CYP2D6 PM patients to receive half of the dose that CYP2D6 EM patients receive.

For normal-weight CYP2D6 PM, the disclosure provides for increasing half of the usual dose by at least 10%, at least about 10%, e.g., about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195%, or about 200%.

In embodiments, the dose of brexpiprazole administering during treatment initiation is increased by at least about 0.125 mg, e.g., about 0.125 mg, about 0.25 mg, about 0.50 mg, about 0.75 mg, about 1.0 mg, about 1.25 mg, 1.5 mg, about 1.75 mg, about 2.0 mg, about 2.5 mg, about 2.75 mg, about 3.0 mg, about 3.25 mg, about 3.5 mg, about 3.75 mg, about 4.0 mg, about 4.25 mg, about 4.5 mg, 4.75 mg, about 5.0 mg, about 5.25 mg, about 5.5 mg, about 5.75 mg, about 6.0 mg, about 6.25 mg, about 6.5 mg, about 6.75 mg, about 7.0 mg, about 7.25 mg, about 7.5 mg, or about 7.75 mg, or about 8.0 mg, inclusive of all values and ranges therein, compared to the dosage administered on the same day of treatment initiation according to the brexpiprazole FDA label. In embodiments, the dose of brexpiprazole administering during treatment initiation is increased by at least about 0.125 mg, 0.25 mg, 0.5 mg, 0.57 mg, or 1 mg.

In embodiments, the dose of brexpiprazole administering during treatment initiation is about 0.25 mg, 0.375 mg, about 0.50 mg, about 0.625 mg, about 0.75 mg, 0.875 mg, about 1.0 mg, about 1.125 mg, about 1.25 mg, about 1.375 mg, 1.5 mg, about 1.625 mg, about 1.75 mg, about 1.875 mg, about 2.0 mg, about 2.125 mg, about 2.25 mg, about 2.375 mg, about 2.5 mg, about 2.625 mg, about 2.75 mg, about 2.875 mg, about 3.0 mg, about 3.125 mg, about 3.25 mg, about 3.325 mg, about 3.5 mg, about 3.625 mg, about 3.75 mg, about 3.875 mg, about 4.0 mg, about 4.125 mg, about 4.25 mg, about 4.375 mg, about 4.5 mg, about 4.625 mg, 4.75 mg, about 4.875 mg, about 5.0 mg, about 5.125 mg, about 5.25 mg, about 5.375 mg, about 5.5 mg, about 5.675 mg, about 5.75 mg, about 5.875 mg, about 6.0 mg, about 6.25 mg, about 6.5 mg, about 6.75 mg, about 7.0 mg, about 7.25 mg, about 7.5 mg, or about 7.75 mg, or about 8.0 mg, inclusive of all values and ranges therein.

Dosage Regimens

The various dosing methods of the present invention, as described herein, comprise initiating treatment by administering an elevated daily dose of brexpiprazole for one or more defined time periods (during treatment initiation), then at an appropriate time (as described herein), administering the FDA-recommended dose appropriate for the indication (e.g., schizophrenia or major depressive disorder). In embodiments, the initial, elevated daily dose is administered once daily (QD) in an amount that brings the blood plasma concentrations closer to the expected concentrations while also avoiding exposure levels (e.g, sharp “peaks” in the plasma levels) that could cause serious side effects such as akathisia. Typically, the daily brexpiprazole doses administered at the initiation of brexpiprazole treatment provide a daily dose that is at least 110% (e.g., 125% or 150%) of the Starting Dose” and/or “Recommended Dose”, or at least 0.125 mg greater (e.g., 0.25, 0.5, 0.75 mg, or 1 mg greater) than the Starting Dose” and/or “Recommended Dose” of brexpiprazole described in the REXULTI® label. The guiding principle for initiating administration of brexpiprazole according to the methods disclosed herein is to increase the daily dose of brexpiprazole for a defined period (i.e., during the treatment initiation period) such that obese CYP2D6 EM or obese CYP2D6 PM patients reach therapeutic plasma levels of brexpiprazole more quickly than would be obtained for such patients using the dosing regimens provided in FDA-approved brexpiprazole labels prior to the present invention. Further, the use of increased daily doses during treatment initiation (e.g., increased relative to the daily “Starting Dose” and/or “Recommended Dose” provided in REXULTI® labels published prior to the present invention) are designed to ensure that no single dose elevates the brexpiprazole plasma levels of such patients to levels which would increase the risk of serious side effects such as akathisia. The limited duration of such increased dosing prior to reverting to the recommended or usual maintenance dose of brexpiprazole is also designed to prevent elevated plasma levels of brexpiprazole that could increase the risk of serious side effects such as akathisia.

The skilled artisan understands that in various embodiments, the magnitude and/or number of initial doses of brexpiprazole can be varied, along with the duration of the initial dosing period, such that the obese patients (EM and PM) according to the present invention reach therapeutic plasma levels of brexpiprazole more rapidly than they would if using the dosing regimens provided in FDA-approved brexpiprazole labels prior to the present invention, without increasing the risk of serious side effects.

The skilled artisan understands that the REXULTI® label (March 2020) contains provisions for dose adjustments in the case of concomitant use with a strong CYP2D6 or strong CYP3A4 inhibitor (e.g., administer half of the dose), or concomitant use with a strong CYP2D6 and strong CYP3A4 inhibitor (e.g., administer a quarter of the dose). These dose adjustments are applied to any relevant recommended dose or patient population.

In some embodiments, the modified dosage regimens of the present invention provide a starting dose. As used herein, a “starting dose” is the lowest dose of brexpiprazole that is administered when initiating treatment with brexpiprazole. In some embodiments, the starting dose is administered on day 1 of brexpiprazole treatment. In some embodiments, the starting dose is administered on days 1-4 of brexpiprazole treatment. In some embodiments, the starting dose is administered on days 1-7 of brexpiprazole treatment. In some embodiments, the starting dose of the modified brexpiprazole dosage regimens of the present disclosure is at least 10% greater (e.g., about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, about 125%, about 150%, about 175%, about 200%, about 225%, about 250%, about 275%, or about 300% or more) than the starting dose instructed by the brexpiprazole (REXULTI®) FDA label (December 2021) for that patient. In some embodiments, the starting dose of the modified brexpiprazole dosage regimen is at least 0.125 mg greater (e.g., about 0.125 mg, about 0.25 mg, about 0.5 mg, about 0.75 mg, about 1.0 mg, about 1.25 mg, or about 1.5 mg or more) than the starting dose instructed by the brexpiprazole (REXULTI®) FDA label (December 2021) for that patient. In some embodiments, the starting dose on days 1-4 of the modified brexpiprazole dosage regimen is at least 10% greater (e.g., about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 125%, about 150%, about 175%, about 200%, about 225%, about 250%, about 275%, or about 300% or more) or at least 0.125 mg greater (e.g., about 0.125 mg, about 0.25 mg, about 0.5 mg, about 0.75 mg, about 1.0 mg or more) than the starting dose instructed by the brexpiprazole (REXULTI®) FDA label (March 2020) for that patient on days 1-4. In some embodiments, the starting dose on days 1-7 of the modified brexpiprazole dosage regimen is at least 10% greater (e.g., about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 125%, about 150%, about 175%, about 200%, about 225%, about 250%, about 275%, or about 300% or more) or at least mg greater (e.g., about 0.125 mg, about 0.25 mg, about 0.5 mg, about 0.75 mg, about 1.0 mg or more) than the starting dose instructed by the brexpiprazole (REXULTI®) FDA label (December 2021) for that patient on days 1-7. In embodiments, the starting dose of brexpiprazole administering on days 1-4 (for schizophrenia) or days 1-7 (for MDD) is about 0.25 mg, 0.375 mg, about 0.50 mg, about 0.625 mg, about 0.75 mg, 0.875 mg, about 1.0 mg, about 1.125 mg, about 1.25 mg, about 1.375 mg, 1.5 mg, about 1.625 mg, about 1.75 mg, about 1.875 mg, about 2.0 mg, inclusive of all values and ranges therein. As used herein, phrases such as “days 1-4”, “days 5-7”, “days 1-7” and the like refer to days after first initiating the administration of brexpiprazole. That is, “day 1” is the first day brexpiprazole is administered to the patient upon initiating treatment, day 7 is the seventh day of brexpiprazole treatment, etc. Initiating brexpiprazole treatment can refer to the first administration to a brexpiprazole-naive patient who has never been administered brexpiprazole, or to a patient who may have been administered brexpiprazole in the past, but has ceased treatment with brexpiprazole for a period sufficient to require re-introduction of brexpiprazole with a lower starting dose of brexpiprazole before increasing to the recommended dose.

In some embodiments, the dose of brexpiprazole is increased from the starting dose. In some embodiments, the dose of brexpiprazole is increased every day, every 2 days, every 2-3 days, every 3-4 days, every 4-5 days, or every 6-7 days, or combinations thereof, during treating initiation. In some embodiments, the dose of brexpiprazole is increased every 3-4 days. In some embodiments, the dose of brexpiprazole is increased every 2 days during initiation. In some embodiments, the brexpiprazole dose is increased every 3 days during treatment initiation. In some embodiments, the dose of brexpiprazole is increased every week, every two weeks, every three weeks, or every month. In some embodiments, the dose of brexpiprazole is increased every week during treatment initiation. In some embodiments, the doses are increased by 1.2-3 fold (e.g., 1.2, 1.3, 1.5, 1.67, 1.75, 2, 2.1, 2.2, 2.3, 2.5, 2.67, 2.75, or 3 fold).

In some embodiments, on day 5 of brexpiprazole administration, the dose of brexpiprazole is at least 10% greater (e.g., about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 125%, about 150%, about 175%, about 200%, about 225%, about 250%, about 275%, or about 300% or more) than the dose provided on the FDA label for that patient on day 5.

In some embodiments, on day 5 of brexpiprazole administration, the dose of brexpiprazole is about 0.5 mg, about 0.625, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25, about 3.5 mg, about 3.75 mg, about 4 mg, inclusive of all values and ranges therein.

In some embodiments, on day 8 of brexpiprazole administration, the dose of brexpiprazole is at least 10% greater ((e.g., about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 125%, about 150%, about 175%, about 200%, about 225%, about 250%, about 275%, or about 300% or more) or at least 0.25 mg greater (e.g., about 0.25 mg, about 0.5 mg, about 0.75 mg, about 1.0 mg or more) than the dose provided on the FDA label for that patient on day 8. In some embodiments, on day 8 of brexpiprazole administration, the dose of brexpiprazole is about 0.5 mg, about 0.625, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, inclusive of all values and ranges therein.

In some embodiments, on day 15 of brexpiprazole administration, the dose of brexpiprazole is at least 10% greater (e.g., about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 125%, about 150%, about 175%, about 200%, about 225%, about 250%, about 275%, or about 300% or more) or at least 0.25 mg greater (e.g., about 0.25 mg, about 0.5 mg, about 0.75 mg, about 1.0 mg, about 1.25, or about 1.5 or more) than the dose provided on the FDA label for that patient on day 15. In some embodiments, on day 15 of brexpiprazole administration, the dose of brexpiprazole is about 1 mg, about 1.25 mg, about 1.5 mg, about 1.75 mg, about 2 mg, about 2.25 mg, about 2.5 mg, about 2.75 mg, about 3 mg, about 3.25, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, inclusive of all values and ranges therein.

In some embodiments, the dose administered on days 8-14 or days 8-21 of brexpiprazole administration is at least 10% greater (e.g., about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 125%, about 150%, about 175%, about 200%, about 225%, about 250%, about 275%, or about 300% or more) or at least 0.25 mg greater (e.g., about 0.25 mg, about 0.5 mg, about 0.75 mg, about 1.0 mg, about 1.25, or about 1.5 or more) than the dose provided on the FDA label for that patient on days 8-14 or days 8-21. In some embodiments, on days 8-14 or days 8-21 of brexpiprazole administration, the dose of brexpiprazole is about 1 mg, about 1.25 mg, about 1.5 mg, about 1.75 mg, about 2 mg, about 2.25 mg, about 2.5 mg, about 2.75 mg, about 3 mg, about 3.25, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg inclusive of all values and ranges therein.

Schizophrenia

In some embodiments, an obese CYP2D6 EM patient or obese CYP2D6 PM patient is administered a starting dose ranging from 1.25-2 mg (e.g., 1.25 mg, 1.5 mg, 1.75 mg, or 2 mg, inclusive of all values and ranges therebetween) brexpiprazole once daily dose on days 1-4, according to the modified dosing regimens disclosed herein. In some embodiments, an normal-weight CYP2D6 PM patient is administered a starting dose ranging from 0.625-0.825 mg (e.g., 0.75 mg) brexpiprazole once daily dose on days 1-4, according to the modified dosing regimens disclosed herein.

In some embodiments, on day 5, the dose of brexpiprazole is increased from the starting dose. In some embodiments, on day 5, the starting dose of brexpiprazole is increased by about 25%, about 50%, about 75%, about 100%, about 125%, about 150%, about 175%, about 200%, about 225%, about 250%, about 275%, or about 300%, including all values and ranges therebetween. In some embodiments, on day 5, the starting dose of brexpiprazole is increased by mg, 0.5 mg, 0.75 mg, 1.0 mg, 1.25 mg, 1.5 mg, 1.75 mg, 2.0 mg, 2.25 mg, 2.5 mg, 2.75 mg or 3 mg, including all values and ranges therebetween. In some embodiments, the dose of brexpiprazole administered on day 5 is double the starting dose. In some embodiments, the total daily dose administered on day 5 ranges from 2.125-4 mg (2.5 mg, 2.75 mg, 3 mg, 3.25 mg, 3.5 mg, 3.75 mg, 3.875 mg, or 4 mg, inclusive of all values and ranges therebetween). In some embodiments, the dose of brexpiprazole is increased on day 5 and the dose is maintained for the duration of brexpiprazole use. In some embodiments, the dose of brexpiprazole is increased on day 5, and the increased dose is administered from days 5-7. In some embodiments, the total daily dose administered on days 5-7 ranges from 2.125-4 mg (2.5 mg, 2.75 mg, 3 mg, 3.25 mg, 3.5 mg, 3.75 mg, or 4 mg, inclusive of all values and ranges therebetween).

In some embodiments, the patient resumes administration of the recommended daily dose starting on day 8. The recommended daily dose for extensive CYP2D6 extensive metabolizers (patients who are not CYP2D6 PM) on the FDA label for brexpiprazole is 2-4 mg. In some embodiments, an obese CYP2D6 EM patient is administered 2-4 mg (e.g., 2 mg, 2.25 mg, 2.5 mg, 2.75 mg, 3 mg, 3.25 mg, 3.5 mg, 3.75 mg, or 4 mg, inclusive of all values and ranges therebetween) starting on day 8. According to the FDA label for brexpiprazole, CYP2D6 PM take have of the recommended dose, which is 1-2 mg. In some embodiments, a normal-weight or obese CYP2D6 PM patient is administered 1-2 mg (e.g., 1 mg, 1.25 mg, 1.5 mg, 1.75 mg, or 2 mg, inclusive of all values and ranges therebetween) starting on day 8.

In some embodiments, on day 8, the dose of brexpiprazole is increased from the dose administered on days 5-7. In some embodiments, on day 8, the dose of brexpiprazole is increased by 25%, 50%, 75%, 100%, 125%, 150%, 175%, 200%, 225%, 250%, 275%, or 300%, including all values and ranges therebetween, compared to the dose administered on days 5-7. In some embodiments, on day 8, the dose of brexpiprazole is increased by 0.25 mg, 0.5 mg, 0.75 mg, 1.0 mg, 1.25 mg, 1.5 mg, 1.75 mg, 2.0 mg, 2.25 mg, 2.5 mg, 2.75 mg, 3 mg, 3.25 mg, 3.5 mg, 3.75 mg, or 4 mg, including all values and ranges therebetween, compared to the dose administered on days 5-7. In some embodiments, the dose administered on days 8 is 3-6 mg (e.g., 3 mg, 3.25 mg, 3.5 mg, 3.75 mg, 4 mg, 4.125, 4.25 mg, 4.5 mg, 4.75 mg, 5 mg, 5.25 mg, 5.5 mg, 5.75 mg, or 6 mg inclusive of all values and ranges therebetween. In some embodiments, the dose of brexpiprazole is increased on day 8 and maintained for the duration of brexpiprazole use. In some embodiments, the dose of brexpiprazole is increased on day 8 and the increased dose is administered until day 14 of brexpiprazole use. In some embodiments, the dose administered on days 8-15 is 3-6 mg (e.g., 3 mg, 3.25 mg, 3.5 mg, 3.75 mg, 4 mg, 4.125 mg, 4.25 mg, 4.5 mg, 4.75 mg, 5 mg, 5.25 mg, 5.5 mg, 5.75 mg, or 6 mg inclusive of all values and ranges therebetween.)

In some embodiments, the patient resumes administration of the recommended daily dose starting on day 15 (or half of the recommended dose, if the patient is a CYP2D6 PM). In some embodiments, an obese CYP2D6 EM patient is administered 2-4 mg/day (2 mg, 2.5 mg, 3 mg, 3.5 mg, or 4 mg, inclusive of all values and ranges therebetween) starting on day 16 of brexpiprazole treatment. In some embodiments, a normal-weight or obese CYP2D6 PM is administered 1-2 mg/day (1 mg, 1.25 mg, 1.5 mg, 1.75 mg, or 2 mg, inclusive of all values and ranges therebetween) starting on day 15 of brexpiprazole treatment.

In some embodiments, an obese CYP2D6 EM patient is administered about 1.25-2 mg of brexpiprazole is administered once daily on each of days 1-4, a dose of about 2.5-3 mg once of brexpiprazole is administered once daily on each of days 5-7, about 3.5-6 mg of brexpiprazole once daily on each day of days 8-14, and thereafter returning to the recommended dose of about 2 to about 4 mg once daily starting on day 15.

In some embodiments, an obese CYP2D6 PM patient is administered a dose of brexpiprazole of about 1.25-2 mg of brexpiprazole is administered once daily on each of days 1-4, a dose of about 2.5-3 mg of brexpiprazole once daily on each of days 5-7, about 3.5-6 mg of brexpiprazole once daily on each day of days 8-14, and thereafter returning to the recommended dose of about 1 to about 2 mg once daily starting on day 15.

In some embodiments, normal-weight CYP2D6 PM patient is administered dose of brexpiprazole of about 0.625-1 mg of brexpiprazole once daily on each of days 1-4, a dose of about 1.25-1.75 mg of brexpiprazole once daily on each of days 5-7, about 2.25-3.5 mg of brexpiprazole once daily on each day of days 8-14, and thereafter returning to the recommended dose of about 2 to about 4 mg once daily starting on day 15.

In some embodiments, a patient with schizophrenia is treated with a modified dosage regimen as found in Table 3.

TABLE 3
Dosing Regimens for Schizophrenia
Table 3.1. Modified Percentage-Based Dosing Regimens
for Obese Patients with Schizophrenia

| | CYP2D6 | | | | |
|---|---|---|---|---|---|
| Weight | Status | Days 1-4 | Days 5-7 | Days 8-14 | Days 15+ |
| All (Label) | EM | 1 mg QD | 2 mg QD | 2-4 mg QD | 2-4 mg QD |
| Obese (Modified | EM | 1.5 mg QD | 3 mg QD | 6 mg QD | 2-4 mg QD |
| Dosing Regimen 1) | | | | | |
| Obese (Modified | EM | 1.25 mg QD | 2.5 mg QD | 5 mg QD | 2-4 mg QD |
| Dosing Regimen 2) | | | | | |
| All (Label) | PM | 0.5 mg QD | 1 mg QD | 1-2 mg QD | 1-2 mg QD |
| Obese (Modified | PM | 1.25 mg BID | 2.5 mg BID | 5 mg BID | 1-2 mg QD |
| Dosing Regimen 3) | | | | | |
| Obese (Modified | PM | 1.5 mg BID | 3 mg BID | 6 mg BID | 1-2 mg QD |
| Dosing Regimen 4) | | | | | |

*Bold Text indicates differences from instructions on FDA label for brexpiprazole (REXULTI ®)

Modified Dosing Regimen 1 represents an increase of 150% in the usual brexpiprazole dosage on each day of days 1-14 compared to the FDA label for brexpiprazole.

Modified Dosing Regimen 2 represents an increase of 125% in the usual brexpiprazole dosage on each day of days 1-14 compared to the FDA label for brexpiprazole.

Modified Dosing Regimen 3 represents an increase of 125% in the usual brexpiprazole dosage on each day of days 1-14 compared to the FDA label for brexpiprazole.

Modified Dosing Regimen 4 represents an increase of 150% in the usual brexpiprazole dosage on each day of days 1-14 compared to the FDA label for brexpiprazole.

TABLE 3.2
Modified Milligram-Based Dosing Regimens for Obese Patients with Schizophrenia

| | CYP2D6 | | | | |
|---|---|---|---|---|---|
| Weight | Status | Days 1-4 | Days 5-7 | Days 8-14 | Days 15+ |
| All (Label) | EM | 1 mg QD | 2 mg QD | 2-4 mg QD | 2-4 mg QD |
| Obese (Modified | EM | 2 mg QD | 3 mg QD | 5 mg QD | 2-4 mg QD |
| Dosing Regimen 5) | | | | | |
| All (Label) | PM | 0.5 mg QD | 1 mg QD | 1-2 mg QD | 1-2 mg QD |
| Obese (Modified | PM | 2 mg QD | 2.5 mg QD | 3.5 mg QD | 1-2 mg QD |
| Dosing Regimen 6) | | | | | |

*Bold Text indicates differences from instructions on FDA label for brexpiprazole (REXULTI ®)

Modified Dosing Regimen 5 represents an increase of 1 mg in the usual brexpiprazole dosage on each day of days 1-14 compared to the FDA label for brexpiprazole.

Modified Dosing Regimen 6 represents an increase of 1.5 mg in the brexpiprazole dosage on each day of days 1-14 compared to the FDA label instructions for CYP2D6 PM patients.

TABLE 3.3
Modified PK-Based Dosing Regimens for Obese Patients with Schizophrenia

| | CYP2D6 | | | | |
|---|---|---|---|---|---|
| Weight | Status | Days 1-4 | Days 5-7 | Days 8-14 | Days 15+ |
| All (Label) | EM | 1 mg QD | 2 mg QD | 2-4 mg QD | 2-4 mg QD |
| Obese (Modified | EM | 1.5 mg QD | 3 mg QD | 5 mg QD | 2-4 mg QD |
| Dosing Regimen 7) | | | | | |
| All (Label) | PM | 0.5 mg QD | 1 mg QD | 1-2 mg QD | 1-2 mg QD |
| Obese (Modified | PM | 1.25 mg QD | 2.5 mg QD | 5 mg QD | 3 mg QD |
| Dosing Regimen 8) | | | | | |

*Bold Text indicates differences from instructions on FDA label for brexpiprazole (REXULTI ®)

Modified Dosing Regimen 7 represents 75% of the usual brexpiprazole dosage on each day of days 1-14 compared to the FDA label for brexpiprazole.

Modified Dosing Regimen 8 represents 0.75 mg increase in the brexpiprazole dosage on each day of days 1-14 compared to the FDA label instructions for a CYP2D6 PM patient.

TABLE 3.4
Modified PK-Based Dosing Regimens for Obese Patients with Schizophrenia

| | CYP2D6 | | | | | Days 10- | Days |
|---|---|---|---|---|---|---|---|
| Weight | Status | Days 1-2 | Days 3-4 | Days 5-7 | Days 8-9 | 14 | 15+ |
| All | EM | 1 mg | 1 mg | 2 mg | 2-4 mg | 2-4 mg | 2-4 mg |
| (Label) | | QD | QD | QD | QD | QD | QD |
| Obese | EM | 1.5 mg | 1.75 mg | 2.5 mg | 3 mg | 3.5 mg | 5 mg |
| (Modified | | QD | QD | QD | QD | QD | QD |
| Dosing | | | | | | | |
| Regimen | | | | | | | |
| 7) | | | | | | | |
| All | PM | 0.5 mg | 0.5 mg | 1 mg | 1-2 mg | 1-2 mg | 1-2 mg |
| (Label) | | QD | QD | QD | QD | QD | QD |
| Obese | PM | 1.25 mg | 1.75 mg | 2.5 mg | 4.5 mg | 5 mg | 3 mg |
| (Modified | | QD | QD | QD | QD | QD | QD |
| Dosing | | | | | | | |
| Regimen | | | | | | | |
| 8) | | | | | | | |

*Bold Text indicates differences from instructions on FDA label for brexpiprazole (REXULTI®)

TABLE 3.5A
Modified Dosing Regimens for Normal-Weight
CYP2D6PM Patients with Schizophrenia

| | CYP2D6 | | | | |
|---|---|---|---|---|---|
| Weight | Status | Days 1-4 | Days 5-7 | Days 8-14 | Days 15+ |
| All (Label) | PM | 0.5 mg QD | 1 mg QD | 1-2 mg QD | 1-2 mg QD |
| Normal-Weight | PM | 0.75 mg QD | 1.5 mg QD | 3 mg QD | 1-2 mg QD |
| (Modified | | | | | |
| Dosing Regimen 9) | | | | | |
| Normal-Weight | PM | 1.25 mg QD | 1.75 mg QD | 2.75 mg QD | 1-2 mg QD |
| (Modified | | | | | |
| Dosing Regimen 10) | | | | | |
| Normal-Weight | PM | 1.25 mg QD | 1.75 mg QD | 2.75 mg QD | 3 mg QD |
| (Modified | | | | | |
| Dosing Regimen 11) | | | | | |

*Bold Text indicates differences from instructions on FDA label for brexpiprazole (REXULTI ®)

Modified Dosing Regimen 9 represents 75% of the usual brexpiprazole dosage on each day of days 1-14 compared to the FDA label for brexpiprazole.

Modified Dosing Regimen 10 represents 0.75 mg increase in the usual brexpiprazole dosage on each day of days 1-14 compared to the FDA label instructions for a CYP2D6 PM patient.

Modified Dosing Regimen 11 represents a PK-based dosing regimen.

TABLE 3.5B
Modified Dosing Regimens for Normal-Weight CYP2D6PM Patients with Schizophrenia

| | CYP2D6 | | | | | Days 10- | Days |
|---|---|---|---|---|---|---|---|
| Weight | Status | Days 1-2 | Days 3-4 | Days 5-7 | Days 8-9 | 14 | 15+ |
| All | PM | 0.5 mg | 0.5 mg | 1 mg | 1-2 mg | 1-2 mg | 1-2 mg |
| (Label) | | QD | QD | QD | QD | QD | QD |
| Normal- | PM | 0.75 mg | 1 mg | 1.25 mg | 3 mg | 3 mg | 3 mg |
| Weight | | QD | QD | QD | QD | QD | QD |
| (Modified | | | | | | | |
| Dosing | | | | | | | |
| Regimen | | | | | | | |
| 11) | | | | | | | |

*Bold Text indicates differences from instructions on FDA label for brexpiprazole (REXULTI®)

Adjunctive Treatment of Major Depressive Disorder (MDD)

In some embodiments, an obese CYP2D6 EM patient or obese CYP2D6 PM patient with MDD is administered a starting dose ranging from 0.625-1.5 mg (e.g., 0.625 mg, mg, 1.0 mg, 1.25 mg, or 1.5 mg, inclusive of all values and ranges therebetween) once daily on days 1-7, according to the modified dosing regimens disclosed herein. In some embodiments, a normal-weight CYP2D6 PM patient with MDD is administered a starting dose ranging from mg (e.g., 0.375 mg, 0.5 mg, 0.75 mg, inclusive of all values and ranges therebetween) once daily on days 1-7, according to the modified dosing regimens disclosed herein.

In some embodiments, on day 8, the dose of brexpiprazole is increased from the starting dose. In some embodiments, on day 8, the starting dose of brexpiprazole is increased by 25%, 50%, 75%, 100%, 125%, 150%, 175%, 200%, 225%, 250%, 275%, or 300%, including all values and ranges therebetween. In some embodiments, on day 8, the starting dose of brexpiprazole is increased by 0.25 mg, 0.5 mg, 0.625 mg, 0.75 mg, 1.0 mg, 1.25 mg, 1.5 mg, 1.75 mg, 2.0 mg, 2.25 mg, 2.5 mg, 2.75 mg, 3 mg, 3.25 mg, 3.5 mg, 3.75 mg, 4 mg, including all values and ranges therebetween. In some embodiments, the dose of brexpiprazole administered on day 8 is 1.3-3 fold greater (e.g., 1.33, 1.67, or 2 fold greater) than the starting dose. In some embodiments, the total daily dose administered on day 8 ranges from 1.25-4 mg (1.25 mg, 1.5 mg, 1.75 mg, 2 mg, 1.25 mg, 1.5 mg, 1.75 mg, 2 mg, 2.25 mg, 2.5 mg, 2.75 mg, 3 mg, 3.25 mg, 3.5 mg, 3.75 mg, or 4 mg, inclusive of all values and ranges therebetween).

In some embodiments, the dose of brexpiprazole is increased on day 8 and the dose is maintained for the duration of brexpiprazole use. In some embodiments, the dose of brexpiprazole is increased on day 8, and the increased dose is administered from days 8-14. In some embodiments, the patient resumes administration of the recommended daily dose starting on day 8 or starting on day 15. In some embodiments, the total daily dose administered on day 8-14 ranges from 1.25-4 mg (1.25 mg, 1.5 mg, 1.75 mg, 2 mg, 1.25 mg, 1.5 mg, 1.75 mg, 2 mg, 2.25 mg, 2.5 mg, 2.75 mg, 3 mg, 3.25 mg, 3.5 mg, 3.75 mg, or 4 mg, inclusive of all values and ranges therebetween).

In some embodiments, the dose of brexpiprazole is increased on day 15, from the dose administered on days 8-14 of brexpiprazole use. In some embodiments, on day 15, the dose of brexpiprazole is increased by 25%, 50%, 75%, 100%, 125%, 150%, 175%, 200%, 225%, 250%, 275%, or 300%, including all values and ranges therebetween. In some embodiments, on day 15, the dose of brexpiprazole administered on days 8-14 is increased by 0.25 mg, 0.5 mg, mg, 0.75 mg, 1.0 mg, 1.25 mg, 1.5 mg, 1.75 mg, 2.0 mg, 2.25 mg, 2.5 mg, 2.75 mg or 3 mg, including all values and ranges therebetween. In some embodiments, the dose of brexpiprazole administered on day 15 is 1.3-3 fold greater (e.g., 1.33, 1.67, or 2 fold) than the dose administered on days 8-14. In some embodiments, the total daily dose administered on day ranges from 1-4 mg (1.0 mg, 1.25 mg, 1.5 mg, 1.75 mg, 2 mg, 1.25 mg, 1.5 mg, 1.75 mg, 2 mg, 2.25 mg, 2.5 mg, 2.75 mg, 3 mg, 3.25 mg, 3.5 mg, 3.75 mg, or 4 mg, inclusive of all values and ranges therebetween).

In some embodiments, patients resume administration of the recommended daily dose starting on day 15. In some embodiments, an obese CYP2D6 EM patient is administered 2-3 mg/day (2 mg, 2.25 mg, 2.5 mg, 2.75 mg, or 3 mg, inclusive of all values and ranges therebetween) starting on day 15 of brexpiprazole treatment. In some embodiments, an obese CYP2D6 PM patient or normal-weight CYP2D6 PM patient is administered 1-1.5 mg/day (1 mg, 1.25 mg, or 1.5 mg, inclusive of all values and ranges therebetween) starting on day 15 of brexpiprazole treatment.

In some embodiments, the dose of brexpiprazole is increased on day 15 from the dose administered on days 8-14 and the dose is maintained for the duration of brexpiprazole use. In some embodiments, the dose of brexpiprazole is increased on day 15 from the dose administered on days 8-14, and the dose is administered on days 15-21 of brexpiprazole treatment. In some embodiments, the total daily dose administered on day 15-21 ranges from 1-4 mg (1.0 mg, 1.25 mg, 1.5 mg, 1.75 mg, 2 mg, 1.25 mg, 1.5 mg, 1.75 mg, 2 mg, 2.25 mg, 2.5 mg, 2.75 mg, 3 mg, 3.25 mg, 3.5 mg, 3.75 mg, or 4 mg, inclusive of all values and ranges therebetween).

In some embodiments, patients resume administration of the recommended daily dose starting on day 22. In some embodiments, an obese CYP2D6 EM patient is administered 2-3 mg/day (2 mg, 2.25 mg, 2.5 mg, 2.75 mg, or 3 mg, inclusive of all values and ranges therebetween) starting on day 22 of brexpiprazole treatment. In some embodiments, a normal weight CYP2D6 PM or obese CYP2D6 EM patient is administered 1-1.5 mg/day (1 mg, 1.25 mg, or 1.5 mg, inclusive of all values and ranges therebetween) starting on day 22 of brexpiprazole treatment.

In some embodiments, an obese CYP2D6 EM patient is administered about 0.625-1.5 mg brexpiprazole once daily on each of the first 7 days of treatment, a dose of 1-3 mg brexpiprazole once daily on each of days 8-14 of treatment, a dose of 2-3 mg brexpiprazole once daily on each of days 15-21, and thereafter returning to the recommended dose of 2-3 mg brexpiprazole once daily.

In some embodiments, an obese CYP2D6 PM patient is administered about 0.625-1.5 mg brexpiprazole once daily on each of the first 7 days of treatment, a dose of 1-3 mg brexpiprazole once daily on each of days 8-14 of treatment, a dose of 2-3 mg brexpiprazole once daily on each of days 15-21, and thereafter returning to the recommended dose of 1-1.5 mg brexpiprazole once daily.

In some embodiments, a normal-weight CYP2D6 PM patient is administered about 0.375-0.75 mg brexpiprazole once daily on each of the first 7 days of treatment, a dose of 0.75-1.5 mg brexpiprazole once daily on each of days 8-14 of treatment, a dose of 1.25-3 mg brexpiprazole once daily on each of days 15-21, and thereafter returning to the recommended dose of 1-1.5 mg brexpiprazole once daily.

In some embodiments, an obese CYP2D6 EM or obese CYP2D6 PM patient with MDD is treated with a modified dosage regimen as found in Table 4.

Table 4. Dosing Regimens for MDD

TABLE 4.1
Modified Percentage-Based Dosing Regimens for MDD

| | CYP2D6 | | | | |
|---|---|---|---|---|---|
| Weight | Status | Days 1-7 | Days 8-14 | Days 15-21 | Days 21+ |
| All (Label) | EM | 0.5 mg QD | 1 mg QD | 2 mg QD | 2-3 mg QD |
| Obese (Modified | EM | 0.75 mg QD | 1.5 mg QD | 2-3 mg QD | 2-3 mg QD |
| Dosing Regimen A) | | | | | |
| Obese (Modified | EM | 0.625 mg QD | 1.25 mg QD | 2.5 mg QD | 2-3 mg QD |
| Dosing Regimen B) | | | | | |
| All (Label) | EM | 1.0 mg QD | 2 mg QD | 2-3 mg QD | 2-3 mg QD |
| Obese (Modified | EM | 1.5 mg QD | 3 mg QD | 2-3 mg QD | 2-3 mg QD |
| Dosing Regimen C) | | | | | |
| Obese (Modified | EM | 1.25 mg QD | 2.5 mg QD | 2.5 mg QD | 2-3 mg QD |
| Dosing Regimen D) | | | | | |
| All (Label) | PM | 0.25 mg QD | 0.5 mg QD | 1.0 mg QD | 1-1.5 mg |
| Obese (Modified | PM | 0.75 mg QD | 1.5 mg QD | 1-1.5 mg QD | 1-1.5 mg QD |
| Dosing Regimen E) | | | | | |
| Obese (Modified | PM | 0.625 mg QD | 1.25 mg QD | 2.5 mg QD | 1-1.5 mg QD |
| Dosing Regimen F) | | | | | |
| All (Label) | PM | 0.5 mg QD | 1 mg QD | 1-1.5 mg QD | 1-1.5 mg QD |
| Obese (Modified | PM | 1.5 mg QD | 3 mg QD | 1-1.5 mg QD | 1-1.5 mg QD |
| Dosing Regimen G) | | | | | |
| Obese (Modified | PM | 1.25 mg QD | 2.5 mg QD | 2.5 mg QD | 1-1.5 mg QD |
| Dosing Regimen H) | | | | | |

*Bold Text indicates differences from instructions on FDA label for brexpiprazole (REXULTI ®)

Modified Dosing Regimen A represents an increase of 150% in the usual brexpiprazole dosage on each day of days 1-14 compared to the FDA label for brexpiprazole.

Modified Dosing Regimen B represents an increase of 125% in the usual brexpiprazole dosage on each day of days 1-21 compared to the FDA label for brexpiprazole.

Modified Dosing Regimen C represents an increase of 150% in the brexpiprazole dosage on each day of days 1-14 compared to the FDA label for brexpiprazole.

Modified Dosing Regimen D represents an increase of 125% in the brexpiprazole dosage on each day of days 1-21 compared to the FDA label for brexpiprazole.

Modified Dosing Regimen E represents an increase of 150% in the brexpiprazole dosage on each day of days 1-14 compared to the FDA label for brexpiprazole.

Modified Dosing Regimen F represents an increase of 125% in the brexpiprazole dosage on each day of days 1-21 compared to the FDA label for brexpiprazole.

Modified Dosing Regimen G represents an increase of 150% in the brexpiprazole dosage on each day of days 1-14 compared to the FDA label for brexpiprazole.

Modified Dosing Regimen H represents an increase of 125% in the brexpiprazole dosage on each day of days 1-21 compared to the FDA label for brexpiprazole.

TABLE 4.2
Modified Milligram-Based Dosing Regimens for MDD

| | CYP2D6 | | | | |
|---|---|---|---|---|---|
| Weight | Status | Days 1-7 | Days 8-14 | Days 15-21 | Days 21+ |
| All (Label) | EM | 0.5 mg QD | 1 mg QD | 2 mg QD | 2-3 mg QD |
| Obese (Modified | EM | 0.75 mg QD | 1.25 mg QD | 2.25 mg QD | 2 mg QD |
| Dosing Regimen I) | | | | | |
| All (Label) | EM | 1.0 mg QD | 2 mg QD | 2-3 mg QD | 2-3 mg QD |
| Obese (Modified | EM | 1.5 mg QD | 2.5 mg QD | 2.5 mg QD | 2-3 mg QD |
| Dosing Regimen J) | | | | | |
| All (Label) | PM | 0.25 mg QD | 0.5 mg QD | 1.0 mg QD | 1-1.5 mg |
| Obese (Modified | PM | 0.75 mg QD | 1 mg QD | 1.5 mg QD | 1-1.5 mg QD |
| Dosing Regimen K) | | | | | |
| All (Label) | PM | 0.5 mg QD | 1 mg QD | 1-1.5 mg QD | 1-1.5 mg QD |
| Obese (Modified | PM | 1.5 mg QD | 2 mg QD | 2 mg QD | 1-1.5 mg QD |
| Dosing Regimen L) | | | | | |

*Bold Text indicates differences from instructions on FDA label for brexpiprazole (REXULTI ®)

Modified Dosing Regimen I represents an increase of 0.25 mg in the usual brexpiprazole dosage on each day of days 1-21 compared to the FDA label for brexpiparzole.

Modified Dosing Regimen J represents an increase of 0.5 mg in the usual brexpiprazole dosage on each day of days 1-21 compared to the FDA label for brexpiprazole.

Modified Dosing Regimen K represents an increase of 0.5 mg in the brexpiprazole dosage on each day of days 1-21 compared to the FDA label instructions for CYP2D6 PM patients.

Modified Dosing Regimen L represents an increase of 1 mg in the brexpiprazole dosage on each day of days 1-21 compared to the FDA label instructions for CYP2D6 PM patients.

TABLE 4.3
Modified PK-Based Dosing Regimens for MDD

| | CYP2D6 | | | | |
|---|---|---|---|---|---|
| Weight | Status | Days 1-7 | Days 8-14 | Days 15-21 | Days 21+ |
| All (Label) | EM | 0.5 mg QD | 1 mg QD | 2-3 mg QD | 2-3 mg QD |
| Obese (Modified | EM | 0.75 mg QD | 1.5 mg QD | 2 mg QD | 2-3 mg QD |
| Dosing Regimen M) | | | | | |
| All (Label) | PM | 0.25 mg QD | 0.5 mg QD | 1-1.5 mg QD | 1-1.5 mg QD |
| Obese (Modified | PM | 0.75 mg QD | 1.25 mg QD | 2 mg QD | 1-1.5 mg QD |
| Dosing Regimen N) | | | | | |

TABLE 4.4
Modified PK-Based Dosing Regimens for MDD

| | CYP2D6 | | | | Days 11- | Days 15- | Days |
|---|---|---|---|---|---|---|---|
| Weight | Status | Days 1-4 | Days 5-7 | Days 8-10 | 14 | 21 | 22+ |
| All | EM | 0.5 mg | 0.5 mg | 1 mg | 1 mg | 2-4 mg | 2-4 mg |
| (Label) | | QD | QD | QD | QD | QD | QD |
| Obese | EM | 0.75 mg | 1 mg | 1.25 mg | 1.5 mg | 1.75 mg | 2 mg |
| (Modified | | QD | QD | QD | QD | QD | QD |
| Dosing | | | | | | | |
| Regimen | | | | | | | |
| 0) | | | | | | | |
| All | PM | 0.25 mg | 0.25 mg | 0.5 mg | 0.5 mg | 1-1.5 mg | 1-1.5 mg |
| (Label) | | QD | QD | QD | QD | QD | QD |
| Obese | PM | 0.5 mg | 0.75 mg | 1 mg | 1.25 mg | 1.75 mg | 1 mg |
| (Modified | | QD | QD | QD | QD | QD | QD |
| Dosing | | | | | | | |
| Regimen | | | | | | | |
| P) | | | | | | | |

*Bold Text indicates differences from instructions on FDA label for brexpiprazole (REXULTI®)

TABLE 4.5A
Modified Dosing Regimens for Normal-Weight CYP2D6PM Patients with MDD

| | CYP2D6 | | | | |
|---|---|---|---|---|---|
| Weight | Status | Days 1-7 | Days 8-14 | Days 15-21 | Days 21+ |
| All (Label) | PM | 0.25 mg QD | 0.5 mg QD | 1-1.5 mg QD | 1-1.5 mg QD |
| Normal-Weight | PM | 0.375 mg QD | 0.75 mg QD | 1.5 mg QD | 1-1.5 mg QD |
| (Modified | | | | | |
| Dosing Regimen Q) | | | | | |
| Normal-Weight | PM | 0.5 mg QD | 0.75 mg QD | 1.25 mg QD | 1-1.5 mg QD |
| (Modified | | | | | |
| Dosing Regimen R) | | | | | |
| Normal-Weight | PM | 0.5 mg QD | 0.75 mg QD | 1.5 mg QD | 1-1.5 mg QD |
| (Modified | | | | | |
| Dosing Regimen U) | | | | | |
| All (Label) | PM | 0.5 mg QD | 1 mg QD | 1-1.5 mg QD | 1-1.5 mg QD |
| Normal-Weight | PM | 0.75 mg QD | 1.5 mg QD | 3 mg QD | 1-1.5 mg QD |
| (Modified | | | | | |
| Dosing Regimen S) | | | | | |
| Normal-Weight | PM | 1 mg QD | 1.5 mg QD | 1.5 mg QD | 1-1.5 mg QD |
| (Modified | | | | | |
| Dosing Regimen T) | | | | | |

*Bold Text indicates differences from instructions on FDA label for brexpiprazole (REXULTI ®)

Modified Dosing Regimen Q represents 75% of the usual brexpiprazole dosage for CYP2D6 EM on each day of days 1-21 compared to the FDA label for brexpiprazole.

Modified Dosing Regimen R represents 0.25 mg increase in the brexpiprazole dosage on each day of days 1-21 compared to the FDA label instructions for CYP2D6 PM patients.

Modified Dosing Regimen S represents 75% of the usual brexpiprazole dosage for CYP2D6 EM on each day of days 1-21 compared to the FDA label for brexpiprazole.

Modified Dosing Regimen T represents 0.75 mg increase in the usual brexpiprazole dosage on each day of days 1-21 compared to the FDA label instructions for CYP2D6 PM patients.

Modified Dosing Regimen U represents a PK-based dosing regimen.

TABLE 4.5B
Modified Dosing Regimens for Normal-Weight CYP2D6PM Patients with MDD

| | CYP2D6 | | | | Days 11- | Days 15- | Days |
|---|---|---|---|---|---|---|---|
| Weight | Status | Days 1-4 | Days 5-7 | Days 8-10 | 14 | 21 | 22+ |
| All | PM | 0.25 mg | 0.25 mg | 0.5 mg | 0.5 mg | 1-1.5 mg | 1-1.5 |
| (Label) | | QD | QD | QD | QD | QD | mg QD |
| Normal | PM | 0.25 mg | 0.5 mg | 0.75 mg | 1.25 mg | 1.5 mg | 1.5 mg |
| Weight | | QD | QD | QD | QD | QD | QD |
| (Modified | | | | | | | |
| Dosing | | | | | | | |
| Regimen | | | | | | | |
| V) | | | | | | | |

*Bold Text indicates differences from instructions on FDA label for brexpiprazole (REXULTI®)

Patient Populations

Applicants have found that certain classes of patients, i.e., obese patients and/or poor hepatic enzyme metabolizers (e.g., CYP2D6 PM), treated with brexpiprazole according to the instructions within the brexpiprazole FDA label (revised December 2021), have substantially lower plasma levels of brexpiprazole when initiating treatment with brexpiprazole, exhibit a substantially longer elimination half-lives (t1/2) of brexpiprazole compared to those exhibited in “normal” patients, and have lower Cₘᵢₙ values than those exhibited in “normal” patients. “Normal” or “normal-weight” patients are patients who do not exhibit the specific physiological characteristics described herein such as BMI of at least about 35 kg/m², % IBW of at least about 150%, waist size greater than about 42 inches, % body fat greater than about 40%, % android body fat greater than about 40%, % gynoid body fat greater than about 40%, total body fat greater than about 40 kg, optionally in combination with impaired hepatic metabolizing enzyme function, e.g., intermediate or poor CYP2D6 metabolizers. Initiating brexpiprazole treatment according to the methods of the disclosure raises the plasma levels of brexpiprazole more quickly to therapeutic levels, and thus increases the Cₘᵢₙ values of brexpiprazole more rapidly to the therapeutic levels obtained by normal patients dosed according to the regimen described in the FDA-approved labels for brexpiprazole published prior to the present invention (e.g., the REXULTI® label dated December 2021).

In some embodiments, the methods of the disclosure are used to treat a patient that is obese. In some embodiments, an obese patient has various characteristics of body fat status (BFS). The term “body fat status,” “body fat characteristics,” “obese status,” “obese characteristics,” “body habitus,” or other derivations or variations thereof refer to at least seven characteristics (BMI, % IBW, waist size, % body fat, % android fat, % gynoid fat, and total body fat) as described herein. In some embodiments, an obese patient can be classified using one or more of the aforementioned BFS. In some embodiments, obese patients exhibit one or more of the following characteristics: BMI of at least about 35 kg/m², % IBW of at least about 150%, waist size greater than about 42 inches, % body fat greater than about 40%, % android body fat greater than about 40%, % gynoid body fat greater than about 40%, total body fat greater than about 40 kg.

In some embodiments, the class of patients treated by the methods of the present disclosure have a body mass index (BMI; expressed in units of kg/m 2 unless otherwise specified) of at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 71, at least about 72, at least about 73, at least about 74, at least about 75, at least about 76, at least about 77, at least about 78, at least about 79, at least about 80, at least about 81, at least about 82, at least about 83, at least about 84, at least about 85, at least about 86, at least about 87, at least about 88, at least about 89, at least about 90, at least about 91, at least about 92, at least about 93, at least about 94, at least about 95, at least about 96, at least about 97, at least about 98, at least about 99, at least about 100, at least about 101, at least about 102, at least about 103, at least about 104, at least about 105, at least about 106, at least about 107, at least about 108, at least about 109, at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, at least about 141, at least about 142, at least about 143, at least about 144, at least about 145, at least about 146, at least about 147, at least about 148, at least about 149, at least about 150, at least about 151, at least about 152, at least about 153, at least about 154, at least about 155, at least about 156, at least about 157, at least about 158, at least about 159, at least about 160, at least about 161, at least about 162, at least about 163, at least about 164, at least about 165, at least about 166, at least about 167, at least about 168, at least about 169, at least about 170, at least about 171, at least about 172, at least about 173, at least about 174, at least about 175, at least about 176, at least about 177, at least about 178, at least about 179, at least about 180, at least about 181, at least about 182, at least about 183, at least about 184, at least about 185, at least about 186, at least about 187, at least about 188, at least about 189, at least about 190, at least about 191, at least about 192, at least about 193, at least about 194, at least about 195, at least about 195, at least about 196, at least about 197, at least about 198, at least about 199, at least about 200, at least about 201, at least about 202, at least about 203, at least about 204, at least about 205, at least about 206, at least about 207, at least about 208, at least about 209, or at least about 210, inclusive of all ranges and subranges therebetween, and any BMI described herein. In one embodiment, the patient has a body mass index (BMI) of at least about 35. In another embodiment, the patient has a body mass index (BMI) of at least about 40. In another embodiment, the patient has a body mass index (BMI) of at least 50.

In some embodiments, a patient treated according to the methods of the present invention has a BMI of at least about 25 to at least about 29.9, at least about 25.5 to at least about 29, at least about 26 to at least about 28.5, at least about 26.5 to at least about 28, or at least about 27 to at least about 27.5, inclusive of all ranges and subranges therebetween, and can be termed overweight or pre-obese. In some embodiments, a patient with a BMI of at least about 30 to at least about 34.9, at least about 30.5 to at least about 34, at least about 31 to at least about 33.5, at least about 31.5 to at least about 33, or at least about 32 to at least about 32.5, inclusive of all ranges and subranges therebetween can be considered obese. In some embodiments, a patient with a BMI of at least about 35 to at least about 39.9, at least about 35.5 to at least about 39, at least about 36 to at least about 38.5, at least about 36.5 to at least about 38, or at least about 37 to at least about 37.5, inclusive of all ranges and subranges therebetween, and any BMI described herein, can be considered obese. In other embodiments, a patient treated by the methods of the present disclosure has a BMI of at least about 35 or more, 40 or more, 50 or more, 60 or more, 70 or more, 80 or more, 90 or more, 100 or more, 110 or more, 120 or more, 130 or more, 140 or more, 150 or more, 160 or more, 170 or more, 180 or more, 190 or more, 200 or more, or 210 or more, inclusive of all ranges and subranges therebetween.

In some embodiments, the patient treated according to the methods of the present disclosure is a child or an adolescent with a BMI of at least about the 85th percentile to at least about 95th percentile, at least about the 86th percentile to at least about 94th percentile, at least about the 87th percentile to at least about 93th percentile, at least about the 88th percentile to at least about 92th percentile, at least about the 89th percentile to at least about 90th percentile, inclusive of all ranges and subranges therebetween, can be considered overweight or pre-obese. In some embodiments, the patient is a patient with a BMI of at least about the 95th percentile, at least about 96th percentile, at least about the 97th percentile, at least about 98th percentile, at least about 99th percentile, or at least about 100th percentile, inclusive of all ranges and subranges therebetween, and any BMI percentile described herein, and can be considered obese. In one embodiment, the patient is about 5 to about 19 years old or about 7 to about 18 years old.

In some embodiments, the patient treated according to the methods of the present disclosure is a female patient in the first trimester through third trimester of a pregnancy and has a BMI of at least 25 to at least about 29.9, at least about 25.5 to at least about 29, at least about 26 to at least about 28.5, at least about 26.5 to at least about 28, or at least about 27 to at least about 27.5, inclusive of all ranges and subranges therebetween, and can be considered overweight or pre-obese. In some embodiments, the patient is a female patient in the first trimester through third trimester of a pregnancy and has a BMI of at least about 30 to at least about 34.9, at least about 30.5 to at least about 34, at least about 31 to at least about 33.5, at least about 31.5 to at least about 33, or at least about 32 to at least about 32.5, inclusive of all ranges and subranges therebetween, and can be considered obese. In some embodiments, the patent treated according to the methods of the present invention is a female patient in the first trimester through third trimester of a pregnancy and has a BMI of at least about 35 to at least about 39.9, at least about to at least about 39, at least about 36 to at least about 38.5, at least about 36.5 to at least about 38, at least about 37 to at least about 37.5, inclusive of all ranges and subranges therebetween, and can be considered severely obese.

In some embodiments, methods of calculating BMI may include, but are not limited to body weight in kilogram/(height in meters)², body weight in pounds/(height in inches)²]×703, and the like.

In some embodiments, the patient treated according to the methods of the present disclosure can alternatively be described as having a % ideal body weight (% IBW) of at least about 110%, at least about 111%, at least about 112%, at least about 113%, at least about 114%, at least about 115%, at least about 116%, at least about 117%, at least about 118%, at least about 119%, at least about 120%, at least about 121%, at least about 122%, at least about 123%, at least about 124%, at least about 125%, at least about 126%, at least about 127%, at least about 128%, at least about 129%, at least about 130%, at least about 131%, at least about 132%, at least about 133%, at least about 134%, at least about 135%, at least about 136%, at least about 137%, at least about 138%, at least about 139%, at least about 140%, at least about 141%, at least about 142%, at least about 143%, at least about 144%, at least about 145%, at least about 146%, at least about 147%, at least about 148%, at least about 149%, at least about 150%, at least about 151%, at least about 152%, at least about 153%, at least about 154%, at least about 155%, at least about 156%, at least about 157%, at least about 158%, at least about 159%, at least about 160%, at least about 161%, at least about 162%, at least about 163%, at least about 164%, at least about 165%, at least about 166%, at least about 167%, at least about 168%, at least about 169%, at least about 170%, at least about 171%, at least about 172%, at least about 173%, at least about 174%, at least about 175%, at least about 176%, at least about 177%, at least about 178%, at least about 179%, at least about 180%, at least about 181%, at least about 182%, at least about 183%, at least about 184%, at least about 185%, at least about 186%, at least about 187%, at least about 188%, at least about 189%, at least about 190%, at least about 191%, at least about 192%, at least about 193%, at least about 194%, at least about 195%, at least about 196%, at least about 197%, at least about 198%, at least about 199%, at least about 200%, at least about 201%, at least about 202%, at least about 203%, at least about 204%, at least about 205%, at least about 206%, at least about 207%, at least about 208%, at least about 209%, at least about 210%, at least about 211%, at least about 212%, at least about 213%, at least about 214%, at least about 215%, at least about 216%, at least about 217%, at least about 218%, at least about 219%, at least about 220%, at least about 221%, at least about 222%, at least about 223%, at least about 224%, at least about 225%, at least about 226%, at least about 227%, at least about 228%, at least about 229%, at least about 230%, at least about 231%, at least about 232%, at least about 233%, at least about 234%, at least about 235%, at least about 236%, at least about 237%, at least about 238%, at least about 239%, at least about 240%, at least about 241%, at least about 242%, at least about 243%, at least about 244%, at least about 245%, at least about 246%, at least about 247%, at least about 248%, at least about 249%, at least about 250%, at least about 251%, at least about 252%, at least about 253%, at least about 254%, at least about 255%, at least about 256%, at least about 257%, at least about 258%, at least about 259%, at least about 260%, at least about 261%, at least about 262%, at least about 263%, at least about 264%, at least about 265%, at least about 266%, at least about 267%, at least about 268%, at least about 269%, at least about 270%, at least about 271%, at least about 272%, at least about 273%, at least about 274%, at least about 275%, at least about 276%, at least about 277%, at least about 278%, at least about 279%, or at least about 280%, inclusive of all ranges and subranges therebetween, and any % ideal body weight described herein. In one embodiment, the patient has % ideal body weight (IBW) of at least about 150%. In one embodiment, the patient has % ideal body weight (IBW) of at least about 250%. In other embodiments, the patient has % IBW of at least 150% and can be considered obese.

In some embodiments, the patient treated according to the present disclosure can alternatively be described as having a waist size or waist circumference greater than about 32, greater than about 33, greater than about 34, greater than about 35 inches, greater than about 36, greater than about 37, greater than about 38, greater than about 39, greater than about 40, greater than about 41, greater than about 42, greater than about 43, greater than about 44, greater than about 45, greater than about 46, greater than about 47, greater than about 48, greater than about 49, greater than about 50, greater than about 51, greater than about 52, greater than about 53, greater than about 54, greater than about 55, greater than about 56, greater than about 57, greater than about 58, greater than about 59, greater than about 60 inches, greater than about 61 inches, greater than about 62 inches, greater than about 63 inches, greater than about 64 inches, greater than about 65 inches, inclusive of all ranges and subranges therebetween, and any waist size or circumference described herein. In one embodiment, a patient having a waist size or waist circumference of about 42 inches can be considered obese. In another embodiment, the patient has waist size or waist circumference greater than about 48 inches. In other embodiments, the patient has waist or waist circumference of at least 42 inches.

In some embodiments, a patient treated according to the methods of the present disclosure has a % body fat greater than about 20%, greater than about 21%, greater than about 22%, greater than about 23%, greater than about 24%, greater than about 25%, greater than about 26%, greater than about 27%, greater than about 28%, greater than about 29%, greater than about 30%, greater than about 31%, greater than about 32%, greater than about 33%, greater than about 34%, greater than about 35%, greater than about 36%, greater than about 37%, greater than about 38%, greater than about 39%, greater than about 40%, greater than about 41%, greater than about 42%, greater than about 43%, greater than about 44%, greater than about 45%, greater than about 46%, greater than about 47%, greater than about 48%, greater than about 49%, or greater than about 50%, inclusive of all ranges and subranges therebetween, and any % body fat described herein. In one embodiment, the patient has a % body fat greater than about 40%. In one embodiment, the patient has a % body fat of at least about 50%. In another embodiment, a patient having a % body fat greater than about 40% can be considered obese. In some embodiments, methods of calculating % body fat can include, but are not limited to total body fat expressed as a percentage of total body weight. Other standards for obesity can be used. For example, the American Council on Exercise suggests that an “average” percentage of body fat for women is about 25-31%, and for men, about 18-24%, and for obese women, about 32% and higher, and obese men, about 25% and higher.

In some embodiments, a patient treated according to the methods of the present disclosure has a % android body fat greater than about 30%, greater than about 31%, greater than about 32%, greater than about 33%, greater than about 34%, greater than about 35%, greater than about 36%, greater than about 37%, greater than about 38%, greater than about 39%, greater than about 40%, greater than about 41%, greater than about 42%, greater than about 43%, greater than about 44%, greater than about 45%, greater than about 46%, greater than about 47%, greater than about 48%, greater than about 49%, greater than about 50%, greater than about 51%, greater than about 52%, greater than about 53%, greater than about 54%, greater than about 55%, greater than about 56%, greater than about 57%, greater than about 58%, greater than about 59%, greater than about 60%, greater than about 61%, greater than about 62%, greater than about 63%, greater than about 64%, greater than about 65%, greater than about 66%, greater than about 67%, greater than about 68%, greater than about 69%, greater than about 70%, greater than about 71%, greater than about 72%, greater than about 73%, greater than about 74%, greater than about 75%, greater than about 76%, greater than about 77%, greater than about 78%, greater than about 79%, or greater than about 80%, inclusive of all ranges and subranges therebetween, and any % android body fat described herein. In one embodiment, a patient having a % android body fat greater than about 40% can be considered obese. In one embodiment, a patient having a % android body fat greater than about 50% can be considered obese.

In some embodiments, a patient treated according to the methods of the present disclosure has a % android body fat of at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, or at least about 80%, inclusive of all ranges and subranges therebetween, and % android body fat described herein. In one embodiment, the patient has % android body fat of at least about 50%.

In some embodiments, a patient treated according to the methods of the present disclosure has a % gynoid body fat greater than about 30%, greater than about 31%, greater than about 32%, greater than about 33%, greater than about 34%, greater than about 35%, greater than about 36%, greater than about 37%, greater than about 38%, greater than about 39%, greater than about 40%, greater than about 41%, greater than about 42%, greater than about 43%, greater than about 44%, greater than about 45%, greater than about 46%, greater than about 47%, greater than about 48%, greater than about 49%, greater than about 50%, greater than about 51%, greater than about 52%, greater than about 53%, greater than about 54%, greater than about 55%, greater than about 56%, greater than about 57%, greater than about 58%, greater than about 59%, greater than about 60%, greater than about 61%, greater than about 62%, greater than about 63%, greater than about 64%, greater than about 65%, greater than about 66%, greater than about 67%, greater than about 68%, greater than about 69%, greater than about 70%, greater than about 71%, greater than about 72%, greater than about 73%, greater than about 74%, greater than about 75%, greater than about 76%, greater than about 77%, greater than about 78%, greater than about 79%, or greater than about 80%, inclusive of all ranges and subranges therebetween, and any % gynoid body fat described herein. In one embodiment, a patient having a % gynoid body fat greater than about 40% can be considered obese. In one embodiment, a patient having a % gynoid body fat greater than about 50% can be considered obese.

In some embodiments, a patient treated according to the methods of the present disclosure has a total body fat content greater than about 30 kg, greater than about 31 kg, greater than about 32 kg, greater than about 33 kg, greater than about 34 kg, greater than about 35 kg, greater than about 36 kg, greater than about 37 kg, greater than about 38 kg, greater than about 39 kg, greater than about 40 kg, greater than about 41 kg, greater than about 42 kg, greater than about 43 kg, greater than about 44 kg, greater than about 45 kg, greater than about 46 kg, greater than about 47 kg, greater than about 48 kg, greater than about 49 kg, greater than about 50 kg, greater than about 51 kg, greater than about 52 kg, greater than about 53 kg, greater than about 54 kg, greater than about 55 kg, greater than about 56 kg, greater than about 57 kg, greater than about 58 kg, greater than about 59 kg, greater than about 60 kg, greater than about 61 kg, greater than about 62 kg, greater than about 63 kg, greater than about 64 kg, greater than about 65 kg, greater than about 66 kg, greater than about 67 kg, greater than about 68 kg, greater than about 69 kg, greater than about 70 kg, greater than about 71 kg, greater than about 72 kg, greater than about 73 kg, greater than about 74 kg, greater than about 75 kg, greater than about 76 kg, greater than about 77 kg, greater than about 78 kg, greater than about 79 kg, greater than about 80 kg, greater than about 81 kg, greater than about 82 kg, greater than about 83 kg, greater than about 84 kg, greater than about 85 kg, greater than about 86 kg, greater than about 87 kg, greater than about 88 kg, greater than about 89 kg, greater than about 90 kg, greater than about 91 kg, greater than about 92 kg, greater than about 93 kg, greater than about 94 kg, greater than about 95 kg, greater than about 96 kg, greater than about 97 kg, greater than about 98 kg, greater than about 99 kg, greater than about 100 kg, at least 101 kg, at least 102 kg, at least 103 kg, at least 104 kg, at least 105 kg, at least 106 kg, at least 107 kg, at least 108 kg, at least 109 kg, or at least 110 kg, inclusive of all ranges and subranges therebetween, and any total body fat described herein. In one embodiment, a patient having total body fat greater than about 40 kg can be considered obese. In one embodiment, a patient having total body fat greater than about 50 kg can be considered obese.

In other embodiments, obesity status of patients treated with the methods of the present disclosure can be measured by waist-to-hip ratio. In other embodiments, obesity status of patients can be measured by skinfold thickness. In other embodiments, obesity status of patients can be measured by bioelectric impedance. In other embodiments, obesity status of patients can be measured by underwater weighing or densitometry. In other embodiments, the obesity status of patients can be measured by air-displacement plethysmography. In other embodiments, obesity status of patients can be measured by dilution method or hydrometry. In other embodiments, the obesity status of patients can be measured by dual energy X-ray absorptiometry. In other embodiments, the obesity status of patients can be measured by computerized tomography and magnetic resonance imaging. In some embodiments, the obesity status can be defined by, but is not limited to adopting the clinical standards, conventional standards, and/or the standards published by the World Health Organization and Center of Disease Control (both of which are herein incorporated by reference in their entireties for all purposes) when using the methods described herein. For example, the WHO defines an obese person as a person with a BMI of 30 or more, an overweight person is one with a BMI equal to or more than 25 (to less than 30). Similarly, the CDC defines normal as a BMI of 18.5 to less than 25, 25.0 to less than 30 as overweight, and 30.0 or higher as obese. The CDC further subdivides obesity into 3 classes: Class 1, a BMI of 30 to less than 35; Class 2, a BMI of 35 to less than 40; and Class 3, as a BMI of 40 or higher. The CDC sometimes refers to Class 3 obesity as “extreme” or “severe” obesity.

In some embodiments, the patient treated by the methods of the present disclosure can be characterized by two or more of the physiological characteristics described herein. For example the patient can have a BMI of at least about 35 and can have a % IBW of at least 150%. In some embodiments, the patient can have a BMI of at least about 35 and can have a waist size greater than about 42 inches. In some embodiments, the patient can have a BMI of at least about and can have a % body fat greater than about 40%. In some embodiments, the patient can have a BMI of at least about 35 and can have a % android body fat greater than about 40%. In some embodiments, the patient can have a BMI of at least about 35 and can have a % gynoid body fat greater than about 40%. In some embodiments, the patient can have a BMI of at least about 35 and can have total body fat greater than about 40 kg. In various other embodiments, the patient can have any combination of two or more of any of the specific physiological parameters described herein.

In some embodiments, the patient can have three or more of the physiological parameters described herein, for example a BMI of at least about 35, a % IBW of at least 150%, and waist size greater than about 42 inches. In some embodiments, the patient can have a BMI of at least about 35, a % IBW of at least 150%, and a % body fat greater than about 40%. In some embodiments, the patient can have a BMI of at least about 35, a % IBW of at least 150%, and a % android body fat greater than about 40%. In some embodiments, the patient can have a BMI of at least about 35, a % IBW of at least 150%, and a % gynoid body fat greater than about 40%. In some embodiments, the patient can have a BMI of at least about 35, a % IBW of at least 150%, and total body fat greater than about 40 kg. In various other embodiments, the patient can have any combination of three or more of any of the specific physiological parameters described herein.

In some embodiments, the patient can have four or more of the physiological parameters described herein, for example the patient can have a BMI of at least about 35, a % IBW of at least 150%, waist size greater than about 42 inches, and a % body fat greater than about 40%. In some embodiments, the patient can have a BMI of at least about 35, a % IBW of at least 150%, waist size greater than about 42 inches, and a % android body fat greater than about 40%. In some embodiments, the patient can have a BMI of at least about 35, a % IBW of at least 150%, waist size greater than about 42 inches, and a % gynoid body fat greater than about 40%. In some embodiments, the patient can have a BMI of at least about 35, a % IBW of at least 150%, a waist size greater than about 42 inches, and total body fat greater than about 43 kg. In some embodiments, the patient can have a BMI of at least about 35, a % IBW of at least 150%, a waist size greater than about 42 inches, a % body fat greater than about 40%, and a % android body fat greater than about 40%. In some embodiments, the patient can have a BMI of at least about 35, a % IBW of at least 150%, a waist size greater than about 42 inches, a % body fat greater than about 40%, and a % gynoid body fat greater than about 40%. In some embodiments, the patient can have a BMI of at least about 35, a % IBW of at least 150%, a waist size greater than about 42 inches, a % body fat greater than about 40%, and total body fat greater than about 40 kg. In some embodiments, the patient can have a BMI of at least about 35, a % IBW of at least 150%, a waist size greater than about 42 inches, a % body fat greater than about 40%, a % android body fat greater than about 40%, in % gynoid body fat greater than about 40%, and total body fat greater than about 40 kg. In one embodiment, the patient who has a BMI of at least about 35, in % IBW of at least 150%, a waist size greater than about 42 inches, and a % body fat greater than about 40%, a % android body fat greater than about 40%, a % gynoid body fat greater than about 40%, and total body fat greater than about 40 kg. In various other embodiments, the patient can have any combination of any or all of the specific physiological parameters described herein.

In some embodiments, the patient can have a waist size greater than about 42 inches, a % body fat greater than about 40%, and a % android body fat greater than about 40%. In some embodiments, the patient can have a waist size greater than about 42 inches, a % body fat greater than about 40%, and a % gynoid body fat greater than about 40%. In some embodiments, the patient can have a waist size greater than about 42 inches, a % body fat greater than about 40%, and total body fat greater than about 40 kg.

In some embodiments, the patient can have a % body fat greater than about 40%, a % android body fat greater than about 40%, and a % gynoid body fat greater than about 40%. In some embodiments, the patient can have a % body fat greater than about 40%, a % android body fat greater than about 40%, and total body fat greater than about 40 kg. In some embodiments, the patient can have a % body fat greater than about 40%, a % gynoid body fat greater than about 40%, and total body fat greater than about 40 kg. In some embodiments, a % android body fat greater than about 40%, and a % gynoid body fat greater than about 40%, and total body fat greater than about 43 kg. In some embodiments, the patient can have any combinations of obesity characteristics described herein.

In some embodiments, the methods of the disclosure are used to treat a normal-weight patient. As used herein, a normal-weight patient has a BMI between about 18 kg/m 2 and kg/m². In some embodiments, normal-weight patients do not exhibit one or more of the following characteristics: BMI of at least about 35 kg/m², % IBW of at least about 150%, waist size greater than about 42 inches, % body fat greater than about 40%, % android body fat greater than about 40%, % gynoid body fat greater than about 40%, total body fat greater than about 40 kg.

In some embodiments, the patient treated by the methods of the present disclosure can be an adult human. In other embodiments, the patient can be a male human. In still other embodiments, the patient can be a female human. In some embodiments, the patient treated by the methods of the present disclosure can be a pediatric human.

In some embodiments, the methods of the disclosure are utilized to treat patients with various hepatic enzyme statuses. Brexpiprazole is metabolized primarily through oxidation via P450 isozymes such as CYP2D6. Alternatively, brexpiprazole is metabolized through oxidation via P450 isozymes such as CYP3A4/5, CYP2C9, CYP2C19, CYP2A6, CYP2C8, or CYP2B6. Each individual may have different activity levels of the P450 isozymes to metabolize brexpiprazole. Categorizations of metabolizers may include, but are not limited to allelic heterogeneity in the P450 isozyme genes. For instance, the CYP2D6 gene can have allelic heterogeneity and its functionality (i.e., associated enzyme activity) can be categorized as full functionality, decreased functionality, and non-functionality. Further, CYP2D6 genotype can be categorized based on its metabolic status by using the “gene dose” method and can have the following scoring scale: (1) alleles with full functionality: a value of 1, (2) alleles with reduced functionality: a value of 0.5, and (3) alleles with no functionality: a value of 0. Alternatively, in some embodiments, the CYP2D6 genotype can be tested by using targeted variant analysis. In some embodiments, the CYP2D6 genotype can be tested by using sequence analysis of select exons.

The “normal” or typical patient has 2 normally functioning CYP2D6 alleles, and has full “normal” CYP2D6 enzyme functionality or activity and is referred to as an “extensive CYP2D6 metabolizer.” Patients with one non-functional CYP2D6 allele and one normally functioning allele have reduced CYP2D6 enzyme function and are termed “intermediate CYP2D6 metabolizers.” Patients with 2 non-functional CYP2D6 alleles have little or no CYP2D6 functionality or activity and are termed “poor CYP2D6 metabolizers” or alternatively “CYP2D6 poor metabolizers (PM).”

As used herein, the term “extensive CYP2D6 metabolizer”, “CYP2D6 extensive metabolizer”, “CYP2D6 EM”, “not a CYP2D6 PM”, and the like, refers to a person who may have a gene dose score for the CYP2D6 allele of 1.5 or 2 and may have superior capabilities for metabolizing brexpiprazole compared to his or her counterpart who is assigned as “intermediate CYP2D6 metabolizer” or “poor CYP2D6 metabolizer.” As used herein, the term “intermediate CYP2D6 metabolizer” refers to a person who may have a gene dose score for the CYP2D6 allele of 0.5 to 1 and may have superior capabilities for metabolizing brexpiprazole compared to his or her counterpart who is assigned as “poor CYP2D6 metabolizer.” As used herein, the term “poor CYP2D6 metabolizer” refers to a person who may have a gene dose score for the CYP2D6 allele of 0 and may have the least capabilities for metabolizing brexpiprazole compared to his or her counterpart who is assigned as an “intermediate metabolizer” or an “extensive metabolizer.” In some embodiments, other suitable or conventional standards of categorizing CYP2D6 metabolizers may be used.

In some embodiments, the methods of the disclosure are used to treat a CYP2D6 poor metabolizer. In some embodiments, the methods of the disclosure are used to treat a CYP2D6 extensive metabolizer. In some embodiments, the methods of the disclosure are used to treat a CYP2D6 intermediate metabolizer.

In some embodiments, the methods of the disclosure are used to treat a patient that is an intermediate CYP2D6 metabolizer and has at least one of the obesity characteristics described herein. In some embodiments, the methods of the disclosure are used to treat a patient that is a poor CYP2D6 metabolizer and has at least one of the obesity characteristics described herein. In some embodiments, the methods of the disclosure are used to treat a patient that is an extensive CYP2D6 metabolizer and has at least one of the obesity characteristics described herein.

In some embodiments, the methods of the disclosure are utilized to treat a patient that is a normal weight and is an intermediate CYP2D6 metabolizer. In some embodiments, the methods of the disclosure are utilized to treat a patient that is a normal weight and is a poor CYP2D6 metabolizer. In some embodiments, the methods of the disclosure are utilized to treat a patient that is a normal weight and is an extensive CYP2D6 metabolizer.

In some embodiments, the methods of the disclosure are utilized to treat a patient that has a BMI greater than 25 kg/m 2 but less than 35 kg/m 2 and is an intermediate CYP2D6 metabolizer. In some embodiments, the methods of the disclosure are utilized to treat a patient that has a BMI greater than 25 kg/m 2 but less than 35 kg/m 2 and is a poor CYP2D6 metabolizer. In some embodiments, the methods of the disclosure are utilized to treat a patient that has a BMI greater than 25 kg/m 2 but less than 35 kg/m 2 and is an extensive CYP2D6 metabolizer.

In some embodiments, the methods of the disclosure are used to treat a patient with a disease selected from major depressive disorder, schizophrenia, post-traumatic stress disorder, bipolar disorder, bipolar I disorder, bipolar depression, acute mania, agitation associated with Alzheimer's disease, borderline personality disorder, attention deficit hyperactivity disorder, autism, irritability associated with Autism Spectrum Disorder, central nervous system disorders, conduct disorder, oppositional defiant disorder, and combinations thereof.

In some embodiments, the methods of the disclosure are used to treat a patient with major depressive disorder. In some embodiments, the methods of the disclosure are used as an adjunctive therapy to treat a patient with major depressive disorder. In some embodiments, the methods of the disclosure are used to treat a patient with schizophrenia.

As used herein, “normal,” “normal-weight,” “reference,” or other derivations or variations thereof refers to a non-obese state in a person who can have at least one of the following characteristics: BMI less than about 35 kg/m 2, % IBW less than about 150%, waist size less than about 42, % body fat less than about 40%, % android body fat less than about 40%, % gynoid body fat less than about 40%, and total body fat less than about 40 kg. Unless otherwise modified “normal metabolizer” also means an extensive CYP2D6 metabolizer.

Pharmacokinetics

Pharmacokinetics Achieved During Initiation: Schizophrenia

In embodiments, the present disclosure provides for methods of achieving one or more of the following median blood plasma concentrations of brexpiprazole:
Day 1: AUC₂₄ of 164 ng*h/mL, Cₘₐₓ of 10 ng/mL, or Cₘᵢₙ of 0 ng/mL;
Day 2: AUC₂₄ of 287 ng*h/mL, Cₘₐₓ of 15 ng/mL, or Cₘᵢₙ of 6 ng/mL;
Day 3: AUC₂₄ of 381 ng*h/mL, Cₘₐₓ of 20 ng/mL, or Cₘᵢₙ of 10 ng/mL;
Day 4: AUC₂₄ of 458 ng*h/mL, Cₘₐₓ of 23 ng/mL, or Cₘᵢₙ of 14 ng/mL;
Day 5: AUC₂₄ of 682 ng*h/mL, Cₘₐₓ of 35 ng/mL, or Cₘᵢₙ of 17 ng/mL;
Day 6: AUC₂₄ of 857 ng*h/mL, Cₘₐₓ of 43 ng/mL, or Cₘᵢₙ of 25 ng/mL;
Day 7: AUC₂₄ of 988 ng*h/mL, Cₘₐₓ of 49 ng/mL, or Cₘᵢₙ of 31 ng/mL;
Day 8: AUC₂₄ of 1432 ng*h/mL, Cₘₐₓ of 73 ng/mL, or Cₘᵢₙ of 36 ng/mL;
Day 9: AUC₂₄ of 1763 ng*h/mL, Cₘₐₓ of 88 ng/mL, or Cₘᵢₙ of 52 ng/mL;
Day 10: AUC₂₄ of 2013 ng*h/mL, Cₘₐₓ of 100 ng/mL, or Cₘᵢₙ of 64 ng/mL;
Day 11: AUC₂₄ of 2226 ng*h/mL, Cₘₐₓ of 107 ng/mL, or Cₘᵢₙ of 73 ng/mL;
Day 12: AUC₂₄ of 2357 ng*h/mL, Cₘₐₓ of 115 ng/mL, or Cₘᵢₙ of 80 ng/mL;
Day 13: AUC₂₄ of 2446 ng*h/mL, Cₘₐₓ of 120 ng/mL, or Cₘᵢₙ of 84 ng/mL;
Day 14: AUC₂₄ of 2506 ng*h/mL, Cₘₐₓ of 124 ng/mL, or Cₘᵢₙ of 88 ng/mL;
Day 15: AUC₂₄ of 2562 ng*h/mL, Cₘₐₓ of 127 ng/mL, or Cₘᵢₙ of 90 ng/mL;
Day 16: AUC₂₄ of 2600 ng*h/mL, Cₘₐₓ of 129 ng/mL, or Cₘᵢₙ of 92 ng/mL;
Day 17: AUC₂₄ of 2635 ng*h/mL, Cₘₐₓ of 130 ng/mL, or Cₘᵢₙ of 93 ng/mL;
Day 18: AUC₂₄ of 2663 ng*h/mL, Cₘₐₓ of 131 ng/mL, or Cₘᵢₙ of 94 ng/mL;
Day 19: AUC₂₄ of 2683 ng*h/mL, Cₘₐₓ of 132 ng/mL, or Cₘᵢₙ of 95 ng/mL;
Day 20: AUC₂₄ of 2701 ng*h/mL, Cₘₐₓ of 132 ng/mL, or Cₘᵢₙ of 97 ng/mL;
Day 21: AUC₂₄ of 2717 ng*h/mL, Cₘₐₓ of 133 ng/mL, or Cₘᵢₙ of 97 ng/mL;
Day 22: AUC₂₄ of 2726 ng*h/mL, Cₘₐₓ of 133 ng/mL, or Cₘᵢₙ of 98 ng/mL;
Day 23: AUC₂₄ of 2732 ng*h/mL, Cₘₐₓ of 133 ng/mL, or Cₘᵢₙ of 98 ng/mL;
Day 24: AUC₂₄ of 2738 ng*h/mL, Cₘₐₓ of 133 ng/mL, or Cₘᵢₙ of 98 ng/mL;
Day 25: AUC₂₄ of 2741 ng*h/mL, Cₘₐₓ of 133 ng/mL, or Cₘᵢₙ of 98 ng/mL;
Day 26: AUC₂₄ of 2743 ng*h/mL, Cₘₐₓ of 133 ng/mL, or Cₘᵢₙ of 98 ng/mL;
Day 27: AUC₂₄ of 2746 ng*h/mL, Cₘₐₓ of 134 ng/mL, or Cₘᵢₙ of 98 ng/mL; and/or
Day 28: AUC₂₄ of 2748 ng*h/mL, Cₘₐₓ of 134 ng/mL, or Cₘᵢₙ of 99 ng/mL.

The above values represent the median blood plasma concentration measured for a population of patients. In embodiments, the median values include a coefficient of variation (% CV) of about 50%, about 45%, about 40%, about 35%, about 30%, about 25%, about 20%, about 15%, or about 10%, including all values and ranges therein. In embodiments, the blood plasma concentrations of brexpiprazole range from about 80% to about 125% of mean values, including the % CV.

In embodiments, the present disclosure provides for methods of achieving one or more of the following blood plasma concentrations (arithmetic mean) of brexpiprazole:
Day 1: AUC₂₄ of 166+/−38 ng*h/mL, Cₘₐₓ of 10+/−2 ng/mL, or Cₘᵢₙ of 0+/−0 ng/mL;
Day 2: AUC₂₄ of 292+/−74 ng*h/mL, Cₘₐₓ of 16+/−3 ng/mL, or Cₘᵢₙ of 6+/−2 ng/mL;
Day 3: AUC₂₄ of 387+/−110 ng*h/mL, Cₘₐₓ of 20+/−5 ng/mL, or Cₘᵢₙ of 11+/−3 ng/mL;
Day 4: AUC₂₄ of 459+/−144 ng*h/mL, Cₘₐₓ of 23+/−6 ng/mL, or Cₘᵢₙ of 14+/−5 ng/mL;
Day 5: AUC₂₄ of 683+/−207 ng*h/mL, Cₘₐₓ of 35+/−9 ng/mL, or Cₘᵢₙ of 17+/−6 ng/mL;
Day 6: AUC₂₄ of 854+/−275 ng*h/mL, Cₘₐₓ of 43+/−11 ng/mL, or Cₘᵢₙ of 25+/−9 ng/mL;
Day 7: AUC₂₄ of 985+/−340 ng*h/mL, Cₘₐₓ of 49+/−14 ng/mL, or Cₘᵢₙ of 31+/−12 ng/mL;
Day 8: AUC₂₄ of 1420+/−458 ng*h/mL, Cₘₐₓ of 73+/−19 ng/mL, or Cₘᵢₙ of 36+/−15 ng/mL;
Day 9: AUC₂₄ of 1753+/−588 ng*h/mL, Cₘₐₓ of 89+/−24 ng/mL, or Cₘᵢₙ of 52+/−20 ng/mL;
Day 10: AUC₂₄ of 2008+/−715 ng*h/mL, Cₘₐₓ of 100+/−30 ng/mL, or Cₘᵢₙ of 64+/−26 ng/mL;
Day 11: AUC₂₄ of 2208+/−833 ng*h/mL, Cₘₐₓ of 109+/−34 ng/mL, or Cₘᵢₙ of 73+/−31 ng/mL;
Day 12: AUC₂₄ of 2366+/−942 ng*h/mL, Cₘₐₓ of 116+/−39 ng/mL, or Cₘᵢₙ of 81+/−36 ng/mL;
Day 13: AUC₂₄ of 2494+/−1041 ng*h/mL, Cₘₐₓ of 122+/−43 ng/mL, or Cₘᵢₙ of 87+/−ng/mL;
Day 14: AUC₂₄ of 2598+/−1131 ng*h/mL, Cₘₐₓ of 127+/−47 ng/mL, or Cₘᵢₙ of 92+/−44 ng/mL;
Day 15: AUC₂₄ of 2684+/−1212 ng*h/mL, Cₘₐₓ of 130+/−50 ng/mL, or Cₘᵢₙ of 95+/−48 ng/mL;
Day 16: AUC₂₄ of 2755+/−1285 ng*h/mL, Cₘₐₓ of 134+/−54 ng/mL, or Cₘᵢₙ of 99+/−51 ng/mL;
Day 17: AUC₂₄ of 2815+/−1351 ng*h/mL, Cₘₐₓ of 136+/−56 ng/mL, or Cₘᵢₙ of 101+/−54 ng/mL;
Day 18: AUC₂₄ of 2866+/−1410 ng*h/mL, Cₘₐₓ of 138+/−59 ng/mL, or Cₘᵢₙ of 104+/−57 ng/mL;
Day 19: AUC₂₄ of 2909+/−1463 ng*h/mL, Cₘₐₓ of 140+/−61 ng/mL, or Cₘᵢₙ of 106+/−59 ng/mL;
Day 20: AUC₂₄ of 2946+/−1512 ng*h/mL, Cₘₐₓ of 142+/−63 ng/mL, or Cₘᵢₙ of 107+/−61 ng/mL;
Day 21: AUC₂₄ of 2978+/−1555 ng*h/mL, Cₘₐₓ of 143+/−65 ng/mL, or Cₘᵢₙ of 109+/−63 ng/mL;
Day 22: AUC₂₄ of 3006+/−1595 ng*h/mL, Cₘₐₓ of 145+/−67 ng/mL, or Cₘᵢₙ of 110+/−ng/mL;
Day 23: AUC₂₄ of 3030+/−1631 ng*h/mL, Cₘₐₓ of 146+/−68 ng/mL, or Cₘᵢₙ of 111+/−66 ng/mL;
Day 24: AUC₂₄ of 3050+/−1663 ng*h/mL, Cₘₐₓ of 147+/−70 ng/mL, or Cₘᵢₙ of 112+/−68 ng/mL;
Day 25: AUC₂₄ of 3069+/−1692 ng*h/mL, Cₘₐₓ of 147+/−71 ng/mL, or Cₘᵢₙ of 113+/−69 ng/mL;
Day 26: AUC₂₄ of 3085+/−1719 ng*h/mL, Cₘₐₓ of 148+/−72 ng/mL, or Cₘᵢₙ of 114+/−ng/mL;
Day 27: AUC₂₄ of 3099+/−1743 ng*h/mL, Cₘₐₓ of 149+/−73 ng/mL, or Cₘᵢₙ of 114+/−71 ng/mL;
Day 28: AUC₂₄ of 3112+/−1766 ng*h/mL, Cₘₐₓ of 149+/−74 ng/mL, or Cₘᵢₙ of 115+/−72 ng/mL.

In embodiments, the blood plasma concentrations (arithmetic mean) of brexpiprazole range from about 80% to about 125% of the above values.

In embodiments, the present disclosure provides for methods of achieving one or more of the following blood plasma (geometric mean) concentrations of brexpiprazole:
Day 1: AUC₂₄ of 161 (25% CV) ng*h/mL, Cₘₐₓ of 10 (26% CV) ng/mL, or Cₘᵢₙ of 1 (0% CV) ng/mL;
Day 2: AUC₂₄ of 282 (27% CV) ng*h/mL, Cₘₐₓ of 15 (23% CV) ng/mL, or Cₘᵢₙ of 6 (30% CV) ng/mL;
Day 3: AUC₂₄ of 370 (31% CV) ng*h/mL, Cₘₐₓ of 19 (25% CV) ng/mL, or Cₘᵢₙ of 10 (35% CV) ng/mL;
Day 4: AUC₂₄ of 436 (35% CV) ng*h/mL, Cₘₐₓ of 22 (28% CV) ng/mL, or Cₘᵢₙ of 13 (40% CV) ng/mL;
Day 5: AUC₂₄ of 650 (33% CV) ng*h/mL, Cₘₐₓ of 34 (27% CV) ng/mL, or Cram of 15 (45% CV) ng/mL;
Day 6: AUC₂₄ of 807 (35% CV) ng*h/mL, Cₘₐₓ of 42 (29% CV) ng/mL, or Cram of 23 (43% CV) ng/mL;
Day 7: AUC₂₄ of 923 (39% CV) ng*h/mL, Cₘₐₓ of 47 (31% CV) ng/mL, or Cram of 29 (45% CV) ng/mL;
Day 8: AUC₂₄ of 1343 (35% CV) ng*h/mL, Cₘₐₓ of 71 (29% CV) ng/mL, or Cram of 33 (49% CV) ng/mL;
Day 9: AUC₂₄ of 1650 (37% CV) ng*h/mL, Cₘₐₓ of 85 (30% CV) ng/mL, or Cram of 48 (45% CV) ng/mL;
Day 10: AUC₂₄ of 1876 (40% CV) ng*h/mL, Cₘₐₓ of 96 (32% CV) ng/mL, or Cram of 58 (47% CV) ng/mL;
Day 11: AUC₂₄ of 2044 (42% CV) ng*h/mL, Cₘₐₓ of 103 (35% CV) ng/mL, or Cₘᵢₙ of 66 (50% CV) ng/mL;
Day 12: AUC₂₄ of 2171 (45% CV) ng*h/mL, Cₘₐₓ of 109 (37% CV) ng/mL, or Cₘᵢₙ of 72 (53% CV) ng/mL;
Day 13: AUC₂₄ of 2269 (47% CV) ng*h/mL, Cₘₐₓ of 114 (39% CV) ng/mL, or Cₘᵢₙ of 77 (56% CV) ng/mL;
Day 14: AUC₂₄ of 2346 (50% CV) ng*h/mL, Cₘₐₓ of 118 (41% CV) ng/mL, or Cram of 80 (59% CV) ng/mL;
Day 15: AUC₂₄ of 2406 (51% CV) ng*h/mL, Cₘₐₓ of 121 (42% CV) ng/mL, or C min of 83 (61% CV) ng/mL;
Day 16: AUC₂₄ of 2454 (53% CV) ng*h/mL, Cₘₐₓ of 123 (44% CV) ng/mL, or Gun of 85 (63% CV) ng/mL;
Day 17: AUC₂₄ of 2493 (54% CV) ng*h/mL, Cₘₐₓ of 125 (45% CV) ng/mL, or Gun of 87 (65% CV) ng/mL;
Day 18: AUC₂₄ of 2525 (56% CV) ng*h/mL, Cₘₐₓ of 126 (46% CV) ng/mL, or Gun of 88 (66% CV) ng/mL;
Day 19: AUC₂₄ of 2551 (57% CV) ng*h/mL, Cₘₐₓ of 127 (47% CV) ng/mL, or Gun of 89 (68% CV) ng/mL;
Day 20: AUC₂₄ of 2573 (58% CV) ng*h/mL, Cₘₐₓ of 129 (48% CV) ng/mL, or Gun of 90 (69% CV) ng/mL;
Day 21: AUC₂₄ of 2591 (59% CV) ng*h/mL, Cₘₐₓ of 129 (49% CV) ng/mL, or Gun of 91 (70% CV) ng/mL;
Day 22: AUC₂₄ of 2606 (59% CV) ng*h/mL, Cₘₐₓ of 130 (49% CV) ng/mL, or Gun of 92 (71% CV) ng/mL;
Day 23: AUC₂₄ of 2619 (60% CV) ng*h/mL, Cₘₐₓ of 131 (50% CV) ng/mL, or Gun of 92 (71% CV) ng/mL;
Day 24: AUC₂₄ of 2630 (60% CV) ng*h/mL, Cₘₐₓ of 131 (51% CV) ng/mL, or Gun of 93 (72% CV) ng/mL;
Day 25: AUC₂₄ of 2639 (61% CV) ng*h/mL, Cₘₐₓ of 132 (51% CV) ng/mL, or Gun of 93 (73% CV) ng/mL;
Day 26: AUC₂₄ of 2647 (61% CV) ng*h/mL, Cₘₐₓ of 132 (51% CV) ng/mL, or Gun of 93 (73% CV) ng/mL;
Day 27: AUC₂₄ of 2654 (62% CV) ng*h/mL, Cₘₐₓ of 132 (52% CV) ng/mL, or Gun of 94 (74% CV) ng/mL;
Day 28: AUC₂₄ of 2660 (62% CV) ng*h/mL, Cₘₐₓ of 133 (52% CV) ng/mL, or Gun of 94 (74% CV) ng/mL.

In embodiments, the blood plasma concentrations of brexpiprazole range from about 80% to about 125% of the above values.

In embodiments, the Day 1 AUC₂₄ of brexpiprazole for 90% of patients treated according to the disclosed methods ranges from 108-235 ng*h/mL. In embodiments, Day 2 AUC₂₄ of brexpiprazole ranges from 182-430 ng*h/mL. In embodiments, Day 3 AUC₂₄ of brexpiprazole ranges from 220-578 ng*h/mL. In embodiments, Day 4 AUC₂₄ of brexpiprazole ranges from 240-698 ng*h/mL. In embodiments, Day 5 AUC₂₄ of brexpiprazole ranges from 369-1026 ng*h/mL. In embodiments, Day 6 AUC₂₄ of brexpiprazole ranges from 447-1284 ng*h/mL. In embodiments, Day 7 AUC₂₄ of brexpiprazole ranges from 480-1515 ng*h/mL. In embodiments, Day 8 AUC₂₄ of brexpiprazole ranges from 738-2129 ng*h/mL. In embodiments, Day 9 AUC₂₄ of brexpiprazole ranges from 893-2673 ng*h/mL. In embodiments, Day 10 AUC₂₄ of brexpiprazole ranges from 960-3137 ng*h/mL. In embodiments, Day 11 AUC₂₄ of brexpiprazole ranges from 997-3541 ng*h/mL. In embodiments, Day 12 AUC₂₄ of brexpiprazole ranges from 1014-3902 ng*h/mL. In embodiments, Day 13 AUC₂₄ of brexpiprazole ranges from 1027-4233 ng*h/mL. In embodiments, Day 14 AUC₂₄ of brexpiprazole ranges from 1035-4526 ng*h/mL. In embodiments, Day 15 AUC₂₄ of brexpiprazole ranges from 1039-4785 ng*h/mL. In embodiments, Day 16 AUC₂₄ of brexpiprazole ranges from 1042-4968 ng*h/mL. In embodiments, Day 17 AUC₂₄ of brexpiprazole ranges from 1043-5152 ng*h/mL. In embodiments, Day 18 AUC₂₄ of brexpiprazole ranges from 1044-5347 ng*h/mL. In embodiments, Day 19 AUC₂₄ of brexpiprazole ranges from 1044-5464 ng*h/mL. In embodiments, Day 20 AUC₂₄ of brexpiprazole ranges from 1044-5636 ng*h/mL. In embodiments, Day 21 AUC₂₄ of brexpiprazole ranges from 1045-5748 ng*h/mL. In embodiments, Day 22 AUC₂₄ of brexpiprazole ranges from 1045-5862 ng*h/mL. In embodiments, Day 23 AUC₂₄ of brexpiprazole ranges from 1045-5973 ng*h/mL. In embodiments, Day 24 AUC₂₄ of brexpiprazole ranges from 1045-6071 ng*h/mL. In embodiments, Day 25 AUC₂₄ of brexpiprazole ranges from 1045-6148 ng*h/mL. In embodiments, Day 26 AUC₂₄ of brexpiprazole ranges from 1045-6183 ng*h/mL. In embodiments, Day 27 AUC₂₄ of brexpiprazole ranges from 1045-6243 ng*h/mL. In embodiments, Day 28 AUC₂₄ of brexpiprazole ranges from 1045-6341 ng*h/mL. In embodiments, the AUC₂₄ of brexpiprazole range from about 80% to about 125% of the above value.

In embodiments, the Day 1 Cₘₐₓ of brexpiprazole for 90% of patients treated according to the disclosed methods ranges from 6-14 ng/mL. In embodiments, Day 2 Cₘₐₓ of brexpiprazole ranges from 10-22 ng/mL. In embodiments, Day 3 Cₘₐₓ of brexpiprazole ranges from 12-28 ng/mL. In embodiments, Day 4 Cₘₐₓ of brexpiprazole ranges from 13-34 ng/mL. In embodiments, Day 5 Cₘₐₓ of brexpiprazole ranges from 20-50 ng/mL. In embodiments, Day 6 Cₘₐₓ of brexpiprazole ranges from 25-63 ng/mL. In embodiments, Day 7 Cₘₐₓ of brexpiprazole ranges from 27-72 ng/mL. In embodiments, Day 8 Cₘₐₓ of brexpiprazole ranges from 42-105 ng/mL. In embodiments, Day 9 Cₘₐₓ of brexpiprazole ranges from 50-128 ng/mL. In embodiments, Day 10 Cₘₐₓ of brexpiprazole ranges from 54-147 ng/mL. In embodiments, Day 11 Cₘₐₓ of brexpiprazole ranges from 59-163 ng/mL. In embodiments, Day 12 Cₘₐₓ of brexpiprazole ranges from 60-178 ng/mL. In embodiments, Day 13 Cₘₐₓ of brexpiprazole ranges from 60-193 ng/mL. In embodiments, Day 14 Cₘₐₓ of brexpiprazole ranges from 60-208 ng/mL. In embodiments, Day 15 Cₘₐₓ of brexpiprazole ranges from 60-216 ng/mL. In embodiments, Day 16 Cₘₐₓ of brexpiprazole ranges from 60-225 ng/mL. In embodiments, Day 17 Cₘₐₓ of brexpiprazole ranges from 60-235 ng/mL. In embodiments, Day 18 Cₘₐₓ of brexpiprazole ranges from 60-240 ng/mL. In embodiments, Day 19 Cₘₐₓ of brexpiprazole ranges from 60-246 ng/mL. In embodiments, Day 20 Cₘₐₓ of brexpiprazole ranges from 60-252 ng/mL. In embodiments, Day 21 Cₘₐₓ of brexpiprazole ranges from 60-257 ng/mL. In embodiments, Day 22 Cₘₐₓ of brexpiprazole ranges from 60-262 ng/mL. In embodiments, Day 23 Cₘₐₓ of brexpiprazole ranges from 60-266 ng/mL. In embodiments, Day 24 Cₘₐₓ of brexpiprazole ranges from 60-270 ng/mL. In embodiments, Day 25 Cₘₐₓ of brexpiprazole ranges from 60-273 ng/mL. In embodiments, Day 26 Cₘₐₓ of brexpiprazole ranges from 60-278 ng/mL. In embodiments, Day 27 Cₘₐₓ of brexpiprazole ranges from 60-282 ng/mL. In embodiments, Day 28 Cₘₐₓ of brexpiprazole ranges from 60-283 ng/mL. In embodiments, the Cₘₐₓ of brexpiprazole range from about 80% to about 125% of the above value.

In embodiments, the Day 1 Cₘᵢₙ of brexpiprazole for 90% of patients treated according to the disclosed methods ranges from 0-0 ng/mL. In embodiments, Day 2 Cₘᵢₙ of brexpiprazole ranges from 4-9 ng/mL. In embodiments, Day 3 Cₘᵢₙ of brexpiprazole ranges from 6-16 ng/mL. In embodiments, Day 4 Cₘᵢₙ of brexpiprazole ranges from 7-22 ng/mL. In embodiments, Day 5 Cₘᵢₙ of brexpiprazole ranges from 7-27 ng/mL. In embodiments, Day 6 Cₘᵢₙ of brexpiprazole ranges from 11-40 ng/mL. In embodiments, Day 7 Cₘᵢₙ of brexpiprazole ranges from 13-51 ng/mL. In embodiments, Day 8 Cₘᵢₙ of brexpiprazole ranges from 14-60 ng/mL. In embodiments, Day 9 Cₘᵢₙ of brexpiprazole ranges from 22-86 ng/mL. In embodiments, Day 10 Cₘᵢₙ of brexpiprazole ranges from 26-107 ng/mL. In embodiments, Day 11 Cₘᵢₙ of brexpiprazole ranges from 28-125 ng/mL. In embodiments, Day 12 Cₘᵢₙ of brexpiprazole ranges from 29-142 ng/mL. In embodiments, Day 13 Cₘᵢₙ of brexpiprazole ranges from 30-154 ng/mL. In embodiments, Day 14 Cₘᵢₙ of brexpiprazole ranges from 30-166 ng/mL. In embodiments, Day 15 Cₘᵢₙ of brexpiprazole ranges from 30-176 ng/mL. In embodiments, Day 16 Cₘᵢₙ of brexpiprazole ranges from 30-185 ng/mL. In embodiments, Day 17 Cₘᵢₙ of brexpiprazole ranges from 31-194 ng/mL. In embodiments, Day 18 Cₘᵢₙ of brexpiprazole ranges from 31-201 ng/mL. In embodiments, Day 19 Cₘᵢₙ of brexpiprazole ranges from 31-209 ng/mL. In embodiments, Day 20 Cₘᵢₙ of brexpiprazole ranges from 31-217 ng/mL. In embodiments, Day 21 Cₘᵢₙ of brexpiprazole ranges from 31-223 ng/mL. In embodiments, Day 22 Cₘᵢₙ of brexpiprazole ranges from 31-227 ng/mL. In embodiments, Day 23 Cₘᵢₙ of brexpiprazole ranges from 31-231 ng/mL. In embodiments, Day 24 Cₘᵢₙ of brexpiprazole ranges from 31-234 ng/mL. In embodiments, Day 25 Cₘᵢₙ of brexpiprazole ranges from 31-237 ng/mL. In embodiments, Day 26 Cₘᵢₙ of brexpiprazole ranges from 31-242 ng/mL. In embodiments, Day 27 Cₘᵢₙ of brexpiprazole ranges from 31-246 ng/mL. In embodiments, Day 28 Cₘᵢₙ of brexpiprazole ranges from 31-249 ng/mL. In embodiments, the Cₘᵢₙ of brexpiprazole range from about 80% to about 125% of the above value.

In embodiments, the AUC₂₄ of brexpiprazole on Days 1-4 ranges from 108-698 ng*h/mL. In embodiments, the AUC₂₄ of brexpiprazole on Days 5-7 ranges from 369-1515 ng*h/mL. In embodiments, the AUC₂₄ of brexpiprazole on Days 8-14 ranges from 738-4526 ng*h/mL. In embodiments, the AUC₂₄ of brexpiprazole on Days 15-28 ranges from 1039-6341 ng*h/mL.

In embodiments, the Cₘₐₓ of brexpiprazole on Days 1-4 ranges from 6-34 ng/mL. In embodiments, the Cₘₐₓ of brexpiprazole on Days 5-7 ranges from 20-72 ng/mL. In embodiments, the Cₘₐₓ of brexpiprazole on Days 8-14 ranges from 42-208 ng/mL. In embodiments, the Cₘₐₓ of brexpiprazole on Days 15-28 ranges from 60-283 ng/mL.

In embodiments, the Gun of brexpiprazole on Days 1-4 ranges from 0-22 ng/mL. In embodiments, the Cₘᵢₙ of brexpiprazole on Days 5-7 ranges from 7-51 ng/mL. In embodiments, the Cₘᵢₙ of brexpiprazole on Days 8-14 ranges from 14-166 ng/mL. In embodiments, the Cₘᵢₙ of brexpiprazole on Days 15-28 ranges from 30-249 ng/mL. Pharmacokinetics Achieved During Initiation: MDD, 0.5 mg Recommended Starting Dose

In embodiments, the present disclosure provides for methods of achieving one or more of the following median blood plasma concentrations of brexpiprazole:
Day 1: AUC₂₄ of 82 ng*h/mL, Cₘₐₓ of 5 ng/mL, or Cₘᵢₙ of 0 ng/mL;
Day 2: AUC₂₄ of 143 ng*h/mL, Cₘₐₓ of 8 ng/mL, or Cₘᵢₙ of 3 ng/mL;
Day 3: AUC₂₄ of 191 ng*h/mL, Cₘₐₓ of 10 ng/mL, or Cₘᵢₙ of 5 ng/mL;
Day 4: AUC₂₄ of 229 ng*h/mL, Cₘₐₓ of 11 ng/mL, or Cₘᵢₙ of 7 ng/mL;
Day 5: AUC₂₄ of 260 ng*h/mL, Cₘₐₓ of 13 ng/mL, or Cₘᵢₙ of 8 ng/mL;
Day 6: AUC₂₄ of 285 ng*h/mL, Cₘₐₓ of 14 ng/mL, or Cₘᵢₙ of 9 ng/mL;
Day 7: AUC₂₄ of 301 ng*h/mL, Cₘₐₓ of 15 ng/mL, or Cₘᵢₙ of 10 ng/mL;
Day 8: AUC₂₄ of 399 ng*h/mL, Cₘₐₓ of 20 ng/mL, or Cₘᵢₙ of 11 ng/mL;
Day 9: AUC₂₄ of 473 ng*h/mL, Cₘₐₓ of 23 ng/mL, or Cₘᵢₙ of 15 ng/mL;
Day 10: AUC₂₄ of 532 ng*h/mL, Cₘₐₓ of 26 ng/mL, or Cₘᵢₙ of 17 ng/mL;
Day 11: AUC₂₄ of 572 ng*h/mL, Cₘₐₓ of 28 ng/mL, or Cₘᵢₙ of 19 ng/mL;
Day 12: AUC₂₄ of 598 ng*h/mL, Cₘₐₓ of 29 ng/mL, or Cₘᵢₙ of 20 ng/mL;
Day 13: AUC₂₄ of 618 ng*h/mL, Cₘₐₓ of 31 ng/mL, or Cₘᵢₙ of 21 ng/mL;
Day 14: AUC₂₄ of 634 ng*h/mL, Cₘₐₓ of 31 ng/mL, or Cₘᵢₙ of 22 ng/mL;
Day 15: AUC₂₄ of 830 ng*h/mL, Cₘₐₓ of 42 ng/mL, or Cₘᵢₙ of 23 ng/mL;
Day 16: AUC₂₄ of 982 ng*h/mL, Cₘₐₓ of 48 ng/mL, or Cₘᵢₙ of 30 ng/mL;
Day 17: AUC₂₄ of 1085 ng*h/mL, Cₘₐₓ of 53 ng/mL, or Cₘᵢₙ of 35 ng/mL;
Day 18: AUC₂₄ of 1149 ng*h/mL, Cₘₐₓ of 57 ng/mL, or Cₘᵢₙ of 38 ng/mL;
Day 19: AUC₂₄ of 1201 ng*h/mL, Cₘₐₓ of 60 ng/mL, or Cₘᵢₙ of 41 ng/mL;
Day 20: AUC₂₄ of 1245 ng*h/mL, Cₘₐₓ of 61 ng/mL, or Cₘᵢₙ of 43 ng/mL;
Day 21 AUC₂₄ of 1271 ng*h/mL, Cₘₐₓ of 63 ng/mL, or Cₘᵢₙ of 45 ng/mL;
Day 22: AUC₂₄ of 1293 ng*h/mL, Cₘₐₓ of 64 ng/mL, or Cₘᵢₙ of 46 ng/mL;
Day 23: AUC₂₄ of 1313 ng*h/mL, Cₘₐₓ of 65 ng/mL, or Cₘᵢₙ of 46 ng/mL;
Day 24: AUC₂₄ of 1328 ng*h/mL, Cₘₐₓ of 65 ng/mL, or Cₘᵢₙ of 47 ng/mL;
Day 25: AUC₂₄ of 1338 ng*h/mL, Cₘₐₓ of 66 ng/mL, or Cₘᵢₙ of 48 ng/mL;
Day 26: AUC₂₄ of 1349 ng*h/mL, Cₘₐₓ of 66 ng/mL, or Cₘᵢₙ of 48 ng/mL;
Day 27: AUC₂₄ of 1356 ng*h/mL, Cₘₐₓ of 66 ng/mL, or Cₘᵢₙ of 49 ng/mL;
Day 28: AUC₂₄ of 1361 ng*h/mL, Cₘₐₓ of 66 ng/mL, or Cₘᵢₙ of 49 ng/mL.

The above values represent the median blood plasma concentration measured for a population of patients. In embodiments, the median values include a coefficient of variation (% CV) of about 50%, about 45%, about 40%, about 35%, about 30%, about 25%, about 20%, about 15%, or about 10%, including all values and ranges therein. In embodiments, the blood plasma concentrations of brexpiprazole range from about 80% to about 125% of the above values, including the % CV.

In embodiments, the present disclosure provides for methods of achieving one or more of the following blood plasma concentrations (arithmetic mean) of brexpiprazole:
Day 1: AUC₂₄ of 83+/−19 ng*h/mL, Cₘₐₓ of 5+/−1 ng/mL, or Cₘᵢₙ of 0+/−0 ng/mL;
Day 2: AUC₂₄ of 146+/−37 ng*h/mL, Cₘₐₓ of 8+/−2 ng/mL, or Gun of 3+/−1 ng/mL;
Day 3: AUC₂₄ of 193+/−55 ng*h/mL, Cₘₐₓ of 10+/−2 ng/mL, or Cₘᵢₙ of 5+/−2 ng/mL;
Day 4: AUC₂₄ of 230+/−72 ng*h/mL, Cₘₐₓ of 12+/−3 ng/mL, or Cₘᵢₙ of 7+/−2 ng/mL;
Day 5: AUC₂₄ of 258+/−89 ng*h/mL, Cₘₐₓ of 13+/−4 ng/mL, or Cₘᵢₙ of 8+/−3 ng/mL;
Day 6: AUC₂₄ of 281+/−104 ng*h/mL, Cₘₐₓ of 14+/−4 ng/mL, or Cₘᵢₙ of 9+/−4 ng/mL;
Day 7: AUC₂₄ of 299+/−117 ng*h/mL, Cₘₐₓ of 15+/−5 ng/mL, or Cₘᵢₙ of 10+/−4 ng/mL;
Day 8: AUC₂₄ of 397+/−143 ng*h/mL, Cₘₐₓ of 20+/−6 ng/mL, or Cₘᵢₙ of 11+/−5 ng/mL;
Day 9: AUC₂₄ of 472+/−172 ng*h/mL, Cₘₐₓ of 24+/−7 ng/mL, or Cₘᵢₙ of 15+/−6 ng/mL;
Day 10: AUC₂₄ of 530+/−202 ng*h/mL, Cₘₐₓ of 26+/−8 ng/mL, or Cₘᵢₙ of 17+/−7 ng/mL;
Day 11: AUC₂₄ of 575+/−229 ng*h/mL, Cₘₐₓ of 28+/−9 ng/mL, or Cₘᵢₙ of 19+/−9 ng/mL;
Day 12: AUC₂₄ of 611+/−254 ng*h/mL, Cₘₐₓ of 30+/−11 ng/mL, or Cₘᵢₙ of 21+/−10 ng/mL;
Day 13: AUC₂₄ of 639+/−277 ng*h/mL, Cₘₐₓ of 31+/−12 ng/mL, or Cₘᵢₙ of 22+/−11 ng/mL;
Day 14: AUC₂₄ of 663+/−298 ng*h/mL, Cₘₐₓ of 32+/−12 ng/mL, or Cₘᵢₙ of 24+/−12 ng/mL;
Day 15: AUC₂₄ of 849+/−341 ng*h/mL, Cₘₐₓ of 43+/−14 ng/mL, or Cₘᵢₙ of 24+/−13 ng/mL;
Day 16: AUC₂₄ of 991+/−393 ng*h/mL, Cₘₐₓ of 49+/−16 ng/mL, or Cₘᵢₙ of 31+/−15 ng/mL;
Day 17: AUC₂₄ of 1099+/−447 ng*h/mL, Cₘₐₓ of 54+/−19 ng/mL, or Cₘᵢₙ of 36+/−17 ng/mL;
Day 18: AUC₂₄ of 1183+/−497 ng*h/mL, Cₘₐₓ of 58+/−21 ng/mL, or Cₘᵢₙ of 40+/−19 ng/mL;
Day 19: AUC₂₄ of 1250+/−544 ng*h/mL, Cₘₐₓ of 61+/−23 ng/mL, or Cₘᵢₙ of 43+/−21 ng/mL;
Day 20: AUC₂₄ of 1304+/−587 ng*h/mL, Cₘₐₓ of 63+/−24 ng/mL, or Cₘᵢₙ of 46+/−23 ng/mL;
Day 21: AUC₂₄ of 1348+/−625 ng*h/mL, Cₘₐₓ of 65+/−26 ng/mL, or Cₘᵢₙ of 48+/−25 ng/mL;
Day 22: AUC₂₄ of 1384+/−660 ng*h/mL, Cₘₐₓ of 67+/−28 ng/mL, or Cₘᵢₙ of 50+/−26 ng/mL;
Day 23: AUC₂₄ of 1414+/−692 ng*h/mL, Cₘₐₓ of 68+/−29 ng/mL, or Cₘᵢₙ of 51+/−28 ng/mL;
Day 24: AUC₂₄ of 1439+/−720 ng*h/mL, Cₘₐₓ of 69+/−30 ng/mL, or Cₘᵢₙ of 52+/−29 ng/mL;
Day 25: AUC₂₄ of 1461+/−746 ng*h/mL, Cₘₐₓ of 70+/−31 ng/mL, or Cₘᵢₙ of 53+/−30 ng/mL;
Day 26: AUC₂₄ of 1479+/−769 ng*h/mL, Cₘₐₓ of 71+/−32 ng/mL, or Cₘᵢₙ of 54+/−31 ng/mL;
Day 27: AUC₂₄ of 1494+/−790 ng*h/mL, Cₘₐₓ of 72+/−33 ng/mL, or Cₘᵢₙ of 55+/−32 ng/mL; or
Day 28: AUC₂₄ of 1508+/−809 ng*h/mL, Cₘₐₓ of 73+/−34 ng/mL, or Cₘᵢₙ of 55+/−33 ng/mL.

In embodiments, the blood plasma concentrations (arithmetic mean) of brexpiprazole range from about 80% to about 125% of the above values.

In embodiments, the present disclosure provides for methods of achieving one or more of the following blood plasma concentrations (geometric mean) of brexpiprazole:
Day 1: AUC₂₄ of 81 (25% CV) ng*h/mL, Cₘₐₓ of 5 (26% CV) ng/mL, or Cₘᵢₙ of 1 (0% CV) ng/mL;
Day 2: AUC₂₄ of 141 (27% CV) ng*h/mL, Cₘₐₓ of 8 (23% CV) ng/mL, or Cram of 3 (30% CV) ng/mL;
Day 3: AUC₂₄ of 185 (31% CV) ng*h/mL, Cₘₐₓ of 10 (25% CV) ng/mL, or Cₘᵢₙ of 5 (35% CV) ng/mL;
Day 4: AUC₂₄ of 218 (35% CV) ng*h/mL, Cₘₐₓ of 11 (28% CV) ng/mL, or Cₘᵢₙ of 7 (40% CV) ng/mL;
Day 5: AUC₂₄ of 242 (39% CV) ng*h/mL, Cₘₐₓ of 12 (31% CV) ng/mL, or Cram of 8 (45% CV) ng/mL;
Day 6: AUC₂₄ of 261 (42% CV) ng*h/mL, Cₘₐₓ of 13 (34% CV) ng/mL, or Cram of 9 (49% CV) ng/mL;
Day 7: AUC₂₄ of 275 (45% CV) ng*h/mL, Cₘₐₓ of 14 (36% CV) ng/mL, or Cram of 9 (53% CV) ng/mL;
Day 8: AUC₂₄ of 371 (40% CV) ng*h/mL, Cₘₐₓ of 19 (32% CV) ng/mL, or Cram of 10 (56% CV) ng/mL;
Day 9: AUC₂₄ of 440 (40% CV) ng*h/mL, Cₘₐₓ of 23 (33% CV) ng/mL, or Cram of 13 (50% CV) ng/mL;
Day 10: AUC₂₄ of 490 (42% CV) ng*h/mL, Cₘₐₓ of 25 (35% CV) ng/mL, or Cₘᵢₙ of 16 (51% CV) ng/mL;
Day 11: AUC₂₄ of 528 (45% CV) ng*h/mL, Cₘₐₓ of 27 (37% CV) ng/mL, or Cₘᵢₙ of 17 (53% CV) ng/mL;
Day 12: AUC₂₄ of 556 (47% CV) ng*h/mL, Cₘₐₓ of 28 (39% CV) ng/mL, or Cₘᵢₙ of 19 (56% CV) ng/mL;
Day 13: AUC₂₄ of 578 (49% CV) ng*h/mL, Cₘₐₓ of 29 (40% CV) ng/mL, or Cₘᵢₙ of 20 (58% CV) ng/mL;
Day 14: AUC₂₄ of 595 (51% CV) ng*h/mL, Cₘₐₓ of 30 (42% CV) ng/mL, or Cram of 20 (60% CV) ng/mL;
Day 15: AUC₂₄ of 780 (44% CV) ng*h/mL, Cₘₐₓ of 40 (36% CV) ng/mL, or Cram of 21 (62% CV) ng/mL;
Day 16: AUC₂₄ of 912 (44% CV) ng*h/mL, Cₘₐₓ of 47 (36% CV) ng/mL, or Cram of 28 (55% CV) ng/mL;
Day 17: AUC₂₄ of 1008 (45% CV) ng*h/mL, Cₘₐₓ of 51 (37% CV) ng/mL, or Cₘᵢₙ of 32 (54% CV) ng/mL;
Day 18: AUC₂₄ of 1078 (47% CV) ng*h/mL, Cₘₐₓ of 54 (38% CV) ng/mL, or Cₘᵢₙ of 36 (56% CV) ng/mL;
Day 19: AUC₂₄ of 1131 (49% CV) ng*h/mL, Cₘₐₓ of 57 (40% CV) ng/mL, or Cₘᵢₙ of 38 (58% CV) ng/mL;
Day 20: AUC₂₄ of 1172 (51% CV) ng*h/mL, Cₘₐₓ of 59 (42% CV) ng/mL, or Cₘᵢₙ of 40 (60% CV) ng/mL;
Day 21: AUC₂₄ of 1204 (52% CV) ng*h/mL, Cₘₐₓ of 60 (43% CV) ng/mL, or Cₘᵢₙ of 41 (62% CV) ng/mL;
Day 22: AUC₂₄ of 1229 (54% CV) ng*h/mL, Cₘₐₓ of 61 (44% CV) ng/mL, or Cₘᵢₙ of 43 (64% CV) ng/mL;
Day 23: AUC₂₄ of 1248 (55% CV) ng*h/mL, Cₘₐₓ of 62 (46% CV) ng/mL, or Cₘᵢₙ of 43 (65% CV) ng/mL;
Day 24: AUC₂₄ of 1265 (56% CV) ng*h/mL, Cₘₐₓ of 63 (47% CV) ng/mL, or Cₘᵢₙ of 44 (67% CV) ng/mL;
Day 25: AUC₂₄ of 1278 (57% CV) ng*h/mL, Cₘₐₓ of 64 (48% CV) ng/mL, or Cₘᵢₙ of 45 (68% CV) ng/mL;
Day 26: AUC₂₄ of 1289 (58% CV) ng*h/mL, Cₘₐₓ of 64 (48% CV) ng/mL, or Cₘᵢₙ of 45 (69% CV) ng/mL;
Day 27: AUC₂₄ of 1297 (59% CV) ng*h/mL, Cₘₐₓ of 65 (49% CV) ng/mL, or Cₘᵢₙ of 46 (70% CV) ng/mL;
Day 28: AUC₂₄ of 1305 (60% CV) ng*h/mL, Cma, of 65 (50% CV) ng/mL, or Cₘᵢₙ of 46 (71% CV) ng/mL.

In embodiments, the blood plasma concentrations of brexpiprazole range from about 80% to about 125% of the above values.

In embodiments, the Day 1 AUC₂₄ of brexpiprazole for 90% of patients treated according to the disclosure ranges from 54-118 ng*h/mL. In embodiments, Day 2 AUC₂₄ of brexpiprazole ranges from 91-215 ng*h/mL. In embodiments, Day 3 AUC₂₄ of brexpiprazole ranges from 110-289 ng*h/mL. In embodiments, Day 4 AUC₂₄ of brexpiprazole ranges from 120-349 ng*h/mL. In embodiments, Day 5 AUC₂₄ of brexpiprazole ranges from 125-396 ng*h/mL. In embodiments, Day 6 AUC₂₄ of brexpiprazole ranges from 127-442 ng*h/mL. In embodiments, Day 7 AUC₂₄ of brexpiprazole ranges from 128-486 ng*h/mL. In embodiments, Day 8 AUC₂₄ of brexpiprazole ranges from 190-625 ng*h/mL. In embodiments, Day 9 AUC₂₄ of brexpiprazole ranges from 225-750 ng*h/mL. In embodiments, Day 10 AUC₂₄ of brexpiprazole ranges from 241-865 ng*h/mL. In embodiments, Day 11 AUC₂₄ of brexpiprazole ranges from 250-952 ng*h/mL. In embodiments, Day 12 AUC₂₄ of brexpiprazole ranges from 254-1037 ng*h/mL. In embodiments, Day 13 AUC₂₄ of brexpiprazole ranges from 257-1113 ng*h/mL. In embodiments, Day 14 AUC₂₄ of brexpiprazole ranges from 259-1180 ng*h/mL. In embodiments, Day 15 AUC₂₄ of brexpiprazole ranges from 382-1429 ng*h/mL. In embodiments, Day 16 AUC₂₄ of brexpiprazole ranges from 449-1654 ng*h/mL. In embodiments, Day 17 AUC₂₄ of brexpiprazole ranges from 483-1854 ng*h/mL. In embodiments, Day 18 AUC₂₄ of brexpiprazole ranges from 499-2037 ng*h/mL. In embodiments, Day 19 AUC₂₄ of brexpiprazole ranges from 508-2187 ng*h/mL. In embodiments, Day 20 AUC₂₄ of brexpiprazole ranges from 514-2314 ng*h/mL. In embodiments, Day 21 AUC₂₄ of brexpiprazole ranges from 518-2434 ng*h/mL. In embodiments, Day 22 AUC₂₄ of brexpiprazole ranges from 520-2545 ng*h/mL. In embodiments, Day 23 AUC₂₄ of brexpiprazole ranges from 521-2622 ng*h/mL. In embodiments, Day 24 AUC₂₄ of brexpiprazole ranges from 522-2709 ng*h/mL. In embodiments, Day 25 AUC₂₄ of brexpiprazole ranges from 522-2780 ng*h/mL. In embodiments, Day 26 AUC₂₄ of brexpiprazole ranges from 522-2841 ng*h/mL. In embodiments, Day 27 AUC₂₄ of brexpiprazole ranges from 522-2906 ng*h/mL. In embodiments, Day 28 AUC₂₄ of brexpiprazole ranges from 522-2964 ng*h/mL. In embodiments, the blood plasma concentrations of brexpiprazole range from about 80% to about 125% of the above values.

In embodiments, the Day 1 Cₘₐₓ of brexpiprazole for 90% of patients treated according to the disclosure ranges from 3-7 ng/mL. In embodiments, Day 2 Cₘₐₓ of brexpiprazole ranges from 5-11 ng/mL. In embodiments, Day 3 Cₘₐₓ of brexpiprazole ranges from 6-14 ng/mL. In embodiments, Day 4 Cₘₐₓ of brexpiprazole ranges from 7-17 ng/mL. In embodiments, Day 5 Cₘₐₓ of brexpiprazole ranges from 7-19 ng/mL. In embodiments, Day 6 Cₘₐₓ of brexpiprazole ranges from 7-21 ng/mL. In embodiments, Day 7 Cₘₐₓ of brexpiprazole ranges from 8-22 ng/mL. In embodiments, Day 8 Cₘₐₓ of brexpiprazole ranges from 11-30 ng/mL. In embodiments, Day 9 Cₘₐₓ of brexpiprazole ranges from 13-35 ng/mL. In embodiments, Day 10 Cₘₐₓ of brexpiprazole ranges from 14-40 ng/mL. In embodiments, Day 11 Cₘₐₓ of brexpiprazole ranges from 15-44 ng/mL. In embodiments, Day 12 Cₘₐₓ of brexpiprazole ranges from 15-48 ng/mL. In embodiments, Day 13 Cₘₐₓ of brexpiprazole ranges from 15-51 ng/mL. In embodiments, Day 14 Cₘₐₓ of brexpiprazole ranges from 15-53 ng/mL. In embodiments, Day 15 Cₘₐₓ of brexpiprazole ranges from 22-67 ng/mL. In embodiments, Day 16 Cₘₐₓ of brexpiprazole ranges from 25-77 ng/mL. In embodiments, Day 17 Cₘₐₓ of brexpiprazole ranges from 28-86 ng/mL. In embodiments, Day 18 Cₘₐₓ of brexpiprazole ranges from 29-93 ng/mL. In embodiments, Day 19 Cₘₐₓ of brexpiprazole ranges from 30-100 ng/mL. In embodiments, Day 20 Cₘₐₓ of brexpiprazole ranges from 30-105 ng/mL. In embodiments, Day 21 Cₘₐₓ of brexpiprazole ranges from 30-111 ng/mL. In embodiments, Day 22 Cₘₐₓ of brexpiprazole ranges from 30-114 ng/mL. In embodiments, Day 23 Cₘₐₓ of brexpiprazole ranges from 30-118 ng/mL. In embodiments, Day 24 Cₘₐₓ of brexpiprazole ranges from 30-122 ng/mL. In embodiments, Day 25 Cₘₐₓ of brexpiprazole ranges from 30-124 ng/mL. In embodiments, Day 26 Cₘₐₓ of brexpiprazole ranges from 30-127 ng/mL. In embodiments, Day 27 Cₘₐₓ of brexpiprazole ranges from 30-130 ng/mL. In embodiments, Day 28 Cₘₐₓ of brexpiprazole ranges from 30-132 ng/mL. In embodiments, the blood plasma concentrations of brexpiprazole range from about 80% to about 125% of the above values.

In embodiments, the Day 1 Cₘᵢₙ of brexpiprazole for 90% of patients treated according to the disclosure ranges from 0-0 ng/mL. In embodiments, Day 2 Cₘᵢₙ of brexpiprazole ranges from 2-5 ng/mL. In embodiments, Day 3 Cₘᵢₙ of brexpiprazole ranges from 3-8 ng/mL. In embodiments, Day 4 Cₘᵢₙ of brexpiprazole ranges from 3-11 ng/mL. In embodiments, Day 5 Cₘᵢₙ of brexpiprazole ranges from 3-13 ng/mL. In embodiments, Day 6 Cₘᵢₙ of brexpiprazole ranges from 4-16 ng/mL. In embodiments, Day 7 Cₘᵢₙ of brexpiprazole ranges from 4-18 ng/mL. In embodiments, Day 8 Cₘᵢₙ of brexpiprazole ranges from 4-19 ng/mL. In embodiments, Day 9 Cₘᵢₙ of brexpiprazole ranges from 6-25 ng/mL. In embodiments, Day 10 Cₘᵢₙ of brexpiprazole ranges from 7-30 ng/mL. In embodiments, Day 11 Cₘᵢₙ of brexpiprazole ranges from 7-34 ng/mL. In embodiments, Day 12 Cₘᵢₙ of brexpiprazole ranges from 7-38 ng/mL. In embodiments, Day 13 Cₘᵢₙ of brexpiprazole ranges from 7-41 ng/mL. In embodiments, Day 14 Cₘᵢₙ of brexpiprazole ranges from 8-43 ng/mL. In embodiments, Day 15 Cₘᵢₙ of brexpiprazole ranges from 8-46 ng/mL. In embodiments, Day 16 Cₘᵢₙ of brexpiprazole ranges from 11-56 ng/mL. In embodiments, Day 17 Cₘᵢₙ of brexpiprazole ranges from 13-65 ng/mL. In embodiments, Day 18 Cₘᵢₙ of brexpiprazole ranges from 14-73 ng/mL. In embodiments, Day 19 Cₘᵢₙ of brexpiprazole ranges from 15-79 ng/mL. In embodiments, Day 20 Cₘᵢₙ of brexpiprazole ranges from 15-85 ng/mL. In embodiments, Day 21 Cₘᵢₙ of brexpiprazole ranges from 15-91 ng/mL. In embodiments, Day 22 Cₘᵢₙ of brexpiprazole ranges from 15-95 ng/mL. In embodiments, Day 23 Cₘᵢₙ of brexpiprazole ranges from 15-99 ng/mL. In embodiments, Day 24 Cₘᵢₙ of brexpiprazole ranges from 15-104 ng/mL. In embodiments, Day 25 Cₘᵢₙ of brexpiprazole ranges from 15-107 ng/mL. In embodiments, Day 26 Cₘᵢₙ of brexpiprazole ranges from 15-110 ng/mL. In embodiments, Day 27 Cₘᵢₙ of brexpiprazole ranges from 15-112 ng/mL. In embodiments, Day 28 Cₘᵢₙ of brexpiprazole ranges from 15-114 ng/mL.

In embodiments, the AUC₂₄ of brexpiprazole on Days 1-7 ranges from 54-486 ng*h/mL. In embodiments, the AUC₂₄ of brexpiprazole on Days 8-14 ranges from 190-1180 ng*h/mL. In embodiments, the AUC₂₄ of brexpiprazole on Days 15-21 ranges from 382-2434 ng*h/mL. In embodiments, the AUC₂₄ of brexpiprazole on Days 22-28 ranges from 520-2964 ng*h/mL.

In embodiments, the Cₘₐₓ of brexpiprazole on Days 1-7 ranges from 3-22 ng/mL. In embodiments, the Cₘₐₓ of brexpiprazole on Days 8-14 ranges from 11-53 ng/mL. In embodiments, the Cₘₐₓ of brexpiprazole on Days 15-21 ranges from 22-111 ng/mL. In embodiments, the Cₘₐₓ of brexpiprazole on Days 22-28 ranges from 30-132 ng/mL.

In embodiments, the Cₘᵢₙ of brexpiprazole on Days 1-7 ranges from 0-18 ng/mL. In embodiments, the Cₘᵢₙ of brexpiprazole on Days 8-14 ranges from 4-43 ng/mL. In embodiments, the Cₘᵢₙ of brexpiprazole on Days 15-21 ranges from 8-91 ng/mL. In embodiments, the Cₘᵢₙ of brexpiprazole on Days 22-28 ranges from 15-114 ng/mL.

Pharmacokinetics Achieved During Initiation: MDD, 1 mg Recommended Starting Dose

In embodiments, the present disclosure provides for methods of achieving one or more of the following median blood plasma concentrations of brexpiprazole:
Day 1: AUC₂₄ of 164 ng*h/mL, Cₘₐₓ of 10 ng/mL, or Cₘᵢₙ of 0 ng/mL;
Day 2: AUC₂₄ of 287 ng*h/mL, Cₘₐₓ of 15 ng/mL, or Cₘᵢₙ of 6 ng/mL;
Day 3: AUC₂₄ of 381 ng*h/mL, Cₘₐₓ of 20 ng/mL, or Cₘᵢₙ of 10 ng/mL;
Day 4: AUC₂₄ of 458 ng*h/mL, Cₘₐₓ of 23 ng/mL, or Cₘᵢₙ of 14 ng/mL;
Day 5: AUC₂₄ of 521 ng*h/mL, Cₘₐₓ of 26 ng/mL, or Cₘᵢₙ of 17 ng/mL;
Day 6: AUC₂₄ of 571 ng*h/mL, Cₘₐₓ of 27 ng/mL, or Cₘᵢₙ of 19 ng/mL;
Day 7: AUC₂₄ of 601 ng*h/mL, Cₘₐₓ of 29 ng/mL, or Cₘᵢₙ of 21 ng/mL;
Day 8: AUC₂₄ of 797 ng*h/mL, Cₘₐₓ of 40 ng/mL, or Cₘᵢₙ of 22 ng/mL;
Day 9: AUC₂₄ of 947 ng*h/mL, Cₘₐₓ of 47 ng/mL, or Cₘᵢₙ of 29 ng/mL;
Day 10: AUC₂₄ of 1063 ng*h/mL, Cₘₐₓ of 52 ng/mL, or Cₘᵢₙ of 34 ng/mL;
Day 11: AUC₂₄ of 1144 ng*h/mL, Cₘₐₓ of 56 ng/mL, or Cₘᵢₙ of 38 ng/mL;
Day 12: AUC₂₄ of 1196 ng*h/mL, Cₘₐₓ of 59 ng/mL, or Cₘᵢₙ of 41 ng/mL;
Day 13: AUC₂₄ of 1236 ng*h/mL, Cₘₐₓ of 61 ng/mL, or Cₘᵢₙ of 43 ng/mL;
Day 14: AUC₂₄ of 1268 ng*h/mL, Cₘₐₓ of 63 ng/mL, or Cₘᵢₙ of 44 ng/mL;
Day 15: AUC₂₄ of 1290 ng*h/mL, Cₘₐₓ of 64 ng/mL, or Cₘᵢₙ of 45 ng/mL;
Day 16: AUC₂₄ of 1308 ng*h/mL, Cₘₐₓ of 65 ng/mL, or Cₘᵢₙ of 46 ng/mL;
Day 17: AUC₂₄ of 1324 ng*h/mL, Cₘₐₓ of 65 ng/mL, or Cₘᵢₙ of 47 ng/mL;
Day 18: AUC₂₄ of 1336 ng*h/mL, Cₘₐₓ of 66 ng/mL, or Cₘᵢₙ of 47 ng/mL;
Day 19: AUC₂₄ of 1346 ng*h/mL, Cₘₐₓ of 66 ng/mL, or Cₘᵢₙ of 48 ng/mL;
Day 20: AUC₂₄ of 1355 ng*h/mL, Cₘₐₓ of 66 ng/mL, or Cₘᵢₙ of 48 ng/mL;
Day 21: AUC₂₄ of 1361 ng*h/mL, Cₘₐₓ of 66 ng/mL, or Cₘᵢₙ of 49 ng/mL;
Day 22: AUC₂₄ of 1364 ng*h/mL, Cₘₐₓ of 66 ng/mL, or Cₘᵢₙ of 49 ng/mL;
Day 23: AUC₂₄ of 1367 ng*h/mL, Cₘₐₓ of 67 ng/mL, or Cₘᵢₙ of 49 ng/mL;
Day 24: AUC₂₄ of 1370 ng*h/mL, Cₘₐₓ of 67 ng/mL, or Cₘᵢₙ of 49 ng/mL;
Day 25: AUC₂₄ of 1371 ng*h/mL, Cₘₐₓ of 67 ng/mL, or Cₘᵢₙ of 49 ng/mL;
Day 26: AUC₂₄ of 1372 ng*h/mL, Cₘₐₓ of 67 ng/mL, or Cₘᵢₙ of 49 ng/mL;
Day 27: AUC₂₄ of 1374 ng*h/mL, Cₘₐₓ of 67 ng/mL, or Cₘᵢₙ of 49 ng/mL; and/or
Day 28: AUC₂₄ of 1374 ng*h/mL, Cₘₐₓ of 67 ng/mL, or Cₘᵢₙ of 49 ng/mL.

The above values represent the median blood plasma concentration measured for a population of patients. In embodiments, the median values include a coefficient of variation (% CV) of about 50%, about 45%, about 40%, about 35%, about 30%, about 25%, about 20%, about 15%, or about 10%, including all values and ranges therein. In embodiments, the blood plasma concentrations of brexpiprazole range from about 80% to about 125% of the above values, including the % CV.

In embodiments, the present disclosure provides for methods of achieving one or more of the following blood plasma concentrations (arithmetic mean) of brexpiprazole:
Day 1: AUC₂₄ of 166+/−38 ng*h/mL, Cₘₐₓ of 10+/−2 ng/mL, or Cₘᵢₙ of 0+/−0 ng/mL;
Day 2: AUC₂₄ of 292+/−74 ng*h/mL, Cₘₐₓ of 16+/−3 ng/mL, or Cₘᵢₙ of 6+/−2 ng/mL;
Day 3: AUC₂₄ of 387+/−110 ng*h/mL, Cₘₐₓ of 20+/−5 ng/mL, or Cₘᵢₙ of 11+/−3 ng/mL;
Day 4: AUC₂₄ of 459+/−144 ng*h/mL, Cₘₐₓ of 23+/−6 ng/mL, or Cₘᵢₙ of 14+/−5 ng/mL;
Day 5: AUC₂₄ of 516+/−177 ng*h/mL, Cₘₐₓ of 26+/−7 ng/mL, or Cₘᵢₙ of 17+/−6 ng/mL;
Day 6: AUC₂₄ of 562+/−207 ng*h/mL, Cₘₐₓ of 28+/−9 ng/mL, or Cₘᵢₙ of 19+/−8 ng/mL;
Day 7: AUC₂₄ of 598+/−235 ng*h/mL, Cₘₐₓ of 29+/−10 ng/mL, or Cₘᵢₙ of 21+/−9 ng/mL;
Day 8: AUC₂₄ of 794+/−286 ng*h/mL, Cₘₐₓ of 40+/−12 ng/mL, or Cₘᵢₙ of 22+/−10 ng/mL;
Day 9: AUC₂₄ of 945+/−345 ng*h/mL, Cₘₐₓ of 47+/−14 ng/mL, or Cₘᵢₙ of 29+/−12 ng/mL;
Day 10: AUC₂₄ of 1060+/−403 ng*h/mL, Cₘₐₓ of 53+/−17 ng/mL, or Cₘᵢₙ of 35+/−15 ng/mL;
Day 11: AUC₂₄ of 1150+/−458 ng*h/mL, Cₘₐₓ of 57+/−19 ng/mL, or Cₘᵢₙ of 39+/−17 ng/mL;
Day 12: AUC₂₄ of 1221+/−508 ng*h/mL, Cₘₐₓ of 60+/−21 ng/mL, or Cₘᵢₙ of 42+/−20 ng/mL;
Day 13: AUC₂₄ of 1279+/−554 ng*h/mL, Cₘₐₓ of 62+/−23 ng/mL, or Cₘᵢₙ of 45+/−22 ng/mL;
Day 14: AUC₂₄ of 1326+/−596 ng*h/mL, Cₘₐₓ of 64+/−25 ng/mL, or Cₘᵢₙ of 47+/−23 ng/mL;
Day 15: AUC₂₄ of 1365+/−633 ng*h/mL, Cₘₐₓ of 66+/−26 ng/mL, or Cₘᵢₙ of 49+/−25 ng/mL;
Day 16: AUC₂₄ of 1397+/−667 ng*h/mL, Cₘₐₓ of 68+/−28 ng/mL, or Cₘᵢₙ of 50+/−27 ng/mL;
Day 17: AUC₂₄ of 1425+/−698 ng*h/mL, Cₘₐₓ of 69+/−29 ng/mL, or Cₘᵢₙ of 52+/−28 ng/mL;
Day 18: AUC₂₄ of 1448+/−725 ng*h/mL, Cₘₐₓ of 70+/−30 ng/mL, or Cₘᵢₙ of 53+/−29 ng/mL;
Day 19: AUC₂₄ of 1467+/−750 ng*h/mL, Cₘₐₓ of 71+/−31 ng/mL, or Cₘᵢₙ of 53+/−30 ng/mL;
Day 20: AUC₂₄ of 1484+/−773 ng*h/mL, Cₘₐₓ of 71+/−32 ng/mL, or Cₘᵢₙ of 54+/−31 ng/mL;
Day 21: AUC₂₄ of 1499+/−793 ng*h/mL, Cₘₐₓ of 72+/−33 ng/mL, or Cₘᵢₙ of 55+/−32 ng/mL;
Day 22: AUC₂₄ of 1511+/−811 ng*h/mL, Cₘₐₓ of 73+/−34 ng/mL, or Cₘᵢₙ of 55+/−33 ng/mL;
Day 23: AUC₂₄ of 1522+/−828 ng*h/mL, Cₘₐₓ of 73+/−35 ng/mL, or Cₘᵢₙ of 56+/−34 ng/mL;
Day 24: AUC₂₄ of 1532+/−843 ng*h/mL, Cₘₐₓ of 74+/−35 ng/mL, or Cram of 56+/−34 ng/mL;
Day 25: AUC₂₄ of 1540+/−857 ng*h/mL, Cₘₐₓ of 74+/−36 ng/mL, or Cₘᵢₙ of 57+/−35 ng/mL;
Day 26: AUC₂₄ of 1548+/−869 ng*h/mL, Cₘₐₓ of 74+/−37 ng/mL, or Cram of 57+/−36 ng/mL;
Day 27: AUC₂₄ of 1554+/−880 ng*h/mL, Cₘₐₓ of 75+/−37 ng/mL, or Cram of 57+/−36 ng/mL; and/or
Day 28: AUC₂₄ of 1560+/−891 ng*h/mL, Cₘₐₓ of 75+/−37 ng/mL, or Cram of 58+/−37 ng/mL.

In embodiments, the blood plasma concentrations of brexpiprazole range from about 80% to about 125% of the above values.

In embodiments, the present disclosure provides for methods of achieving one or more of the following blood plasma concentrations (geometric mean) of brexpiprazole:
Day 1: AUC₂₄ of 161 (25% CV) ng*h/mL, Cₘₐₓ of 10 (26% CV) ng/mL, or Cₘᵢₙ of 1 (0% CV) ng/mL;
Day 2: AUC₂₄ of 282 (27% CV) ng*h/mL, Cₘₐₓ of 15 (23% CV) ng/mL, or Cram of 6 (30% CV) ng/mL;
Day 3: AUC₂₄ of 370 (31% CV) ng*h/mL, Cₘₐₓ of 19 (25% CV) ng/mL, or Cₘᵢₙ of 10 (35% CV) ng/mL;
Day 4: AUC₂₄ of 436 (35% CV) ng*h/mL, Cₘₐₓ of 22 (28% CV) ng/mL, or Cₘᵢₙ of 13 (40% CV) ng/mL;
Day 5: AUC₂₄ of 484 (39% CV) ng*h/mL, Cₘₐₓ of 25 (31% CV) ng/mL, or Cram of 15 (45% CV) ng/mL;
Day 6: AUC₂₄ of 521 (42% CV) ng*h/mL, Cₘₐₓ of 26 (34% CV) ng/mL, or Cₘᵢₙ of 17 (49% CV) ng/mL;
Day 7: AUC₂₄ of 550 (45% CV) ng*h/mL, Cₘₐₓ of 28 (36% CV) ng/mL, or Cₘᵢₙ of 18 (53% CV) ng/mL;
Day 8: AUC₂₄ of 741 (40% CV) ng*h/mL, Cₘₐₓ of 39 (32% CV) ng/mL, or Cₘᵢₙ of 19 (56% CV) ng/mL;
Day 9: AUC₂₄ of 880 (40% CV) ng*h/mL, Cₘₐₓ of 45 (33% CV) ng/mL, or Cₘᵢₙ of 26 (50% CV) ng/mL;
Day 10: AUC₂₄ of 981 (42% CV) ng*h/mL, Cₘₐₓ of 50 (35% CV) ng/mL, or Cram of 31 (51% CV) ng/mL;
Day 11: AUC₂₄ of 1056 (45% CV) ng*h/mL, Cₘₐₓ of 53 (37% CV) ng/mL, or Cₘᵢₙ of 35 (53% CV) ng/mL;
Day 12: AUC₂₄ of 1113 (47% CV) ng*h/mL, Cₘₐₓ of 56 (39% CV) ng/mL, or Cₘᵢₙ of 37 (56% CV) ng/mL;
Day 13: AUC₂₄ of 1156 (49% CV) ng*h/mL, Cₘₐₓ of 58 (40% CV) ng/mL, or Cₘᵢₙ of 39 (58% CV) ng/mL;
Day 14: AUC₂₄ of 1191 (51% CV) ng*h/mL, Cₘₐₓ of 60 (42% CV) ng/mL, or Cₘᵢₙ of 41 (60% CV) ng/mL;
Day 15: AUC₂₄ of 1218 (53% CV) ng*h/mL, Cₘₐₓ of 61 (43% CV) ng/mL, or Cₘᵢₙ of 42 (62% CV) ng/mL;
Day 16: AUC₂₄ of 1239 (54% CV) ng*h/mL, Cₘₐₓ of 62 (45% CV) ng/mL, or Cₘᵢₙ of 43 (64% CV) ng/mL;
Day 17: AUC₂₄ of 1257 (55% CV) ng*h/mL, Cₘₐₓ of 63 (46% CV) ng/mL, or Cₘᵢₙ of 44 (66% CV) ng/mL;
Day 18: AUC₂₄ of 1271 (56% CV) ng*h/mL, Cₘₐₓ of 64 (47% CV) ng/mL, or Cₘᵢₙ of 44 (67% CV) ng/mL;
Day 19: AUC₂₄ of 1283 (57% CV) ng*h/mL, Cₘₐₓ of 64 (48% CV) ng/mL, or Cₘᵢₙ of 45 (68% CV) ng/mL;
Day 20: AUC₂₄ of 1293 (58% CV) ng*h/mL, Cₘₐₓ of 65 (49% CV) ng/mL, or Cₘᵢₙ of 45 (69% CV) ng/mL;
Day 21: AUC₂₄ of 1301 (59% CV) ng*h/mL, Cₘₐₓ of 65 (49% CV) ng/mL, or Cₘᵢₙ of 46 (70% CV) ng/mL;
Day 22: AUC₂₄ of 1308 (60% CV) ng*h/mL, Cₘₐₓ of 65 (50% CV) ng/mL, or Cₘᵢₙ of 46 (71% CV) ng/mL;
Day 23: AUC₂₄ of 1313 (60% CV) ng*h/mL, Cₘₐₓ of 66 (50% CV) ng/mL, or Cₘᵢₙ of 46 (72% CV) ng/mL;
Day 24: AUC₂₄ of 1318 (61% CV) ng*h/mL, Cₘₐₓ of 66 (51% CV) ng/mL, or Cₘᵢₙ of 46 (72% CV) ng/mL;
Day 25: AUC₂₄ of 1322 (61% CV) ng*h/mL, Cₘₐₓ of 66 (51% CV) ng/mL, or Cₘᵢₙ of 47 (73% CV) ng/mL;
Day 26: AUC₂₄ of 1326 (62% CV) ng*h/mL, Cₘₐₓ of 66 (52% CV) ng/mL, or Cₘᵢₙ of 47 (74% CV) ng/mL;
Day 27: AUC₂₄ of 1329 (62% CV) ng*h/mL, Cₘₐₓ of 66 (52% CV) ng/mL, or Cₘᵢₙ of 47 (74% CV) ng/mL;
Day 28: AUC₂₄ of 1332 (62% CV) ng*h/mL, Cₘₐₓ of 66 (52% CV) ng/mL, or Cₘᵢₙ of 47 (74% CV) ng/mL.

In embodiments, the blood plasma concentrations of brexpiprazole range from about 80% to about 125% of the above values.

In embodiments, the Day 1 AUC₂₄ of brexpiprazole for 90% of patients treated according to the disclosure ranges from 108-235 ng*h/mL. In embodiments, Day 2 AUC₂₄ of brexpiprazole ranges from 182-430 ng*h/mL. In embodiments, Day 3 AUC₂₄ of brexpiprazole ranges from 220-578 ng*h/mL. In embodiments, Day 4 AUC₂₄ of brexpiprazole ranges from 240-698 ng*h/mL. In embodiments, Day 5 AUC₂₄ of brexpiprazole ranges from 249-791 ng*h/mL. In embodiments, Day 6 AUC₂₄ of brexpiprazole ranges from 253-885 ng*h/mL. In embodiments, Day 7 AUC₂₄ of brexpiprazole ranges from 257-972 ng*h/mL. In embodiments, Day 8 AUC₂₄ of brexpiprazole ranges from 380-1251 ng*h/mL. In embodiments, Day 9 AUC₂₄ of brexpiprazole ranges from 449-1499 ng*h/mL. In embodiments, Day 10 AUC₂₄ of brexpiprazole ranges from 483-1731 ng*h/mL. In embodiments, Day 11 AUC₂₄ of brexpiprazole ranges from 499-1904 ng*h/mL. In embodiments, Day 12 AUC₂₄ of brexpiprazole ranges from 508-2075 ng*h/mL. In embodiments, Day 13 AUC₂₄ of brexpiprazole ranges from 514-2226 ng*h/mL. In embodiments, Day 14 AUC₂₄ of brexpiprazole ranges from 518-2360 ng*h/mL. In embodiments, Day 15 AUC₂₄ of brexpiprazole ranges from 520-2454 ng*h/mL. In embodiments, Day 16 AUC₂₄ of brexpiprazole ranges from 521-2557 ng*h/mL. In embodiments, Day 17 AUC₂₄ of brexpiprazole ranges from 522-2654 ng*h/mL. In embodiments, Day 18 AUC₂₄ of brexpiprazole ranges from 522-2714 ng*h/mL. In embodiments, Day 19 AUC₂₄ of brexpiprazole ranges from 522-2801 ng*h/mL. In embodiments, Day 20 AUC₂₄ of brexpiprazole ranges from 522-2859 ng*h/mL. In embodiments, Day 21 AUC₂₄ of brexpiprazole ranges from 522-2917 ng*h/mL. In embodiments, Day 22 AUC₂₄ of brexpiprazole ranges from 522-2974 ng*h/mL. In embodiments, Day 23 AUC₂₄ of brexpiprazole ranges from 522-3024 ng*h/mL. In embodiments, Day 24 AUC₂₄ of brexpiprazole ranges from 522-3067 ng*h/mL. In embodiments, Day 25 AUC₂₄ of brexpiprazole ranges from 522-3085 ng*h/mL. In embodiments, Day 26 AUC₂₄ of brexpiprazole ranges from 522-3113 ng*h/mL. In embodiments, Day 27 AUC₂₄ of brexpiprazole ranges from 522-3162 ng*h/mL. In embodiments, Day 28 AUC₂₄ of brexpiprazole ranges from 522-3207 ng*h/mL. In embodiments, the blood plasma concentrations of brexpiprazole range from about 80% to about 125% of the above values.

In embodiments, the Day 1 Cₘₐₓ of brexpiprazole for 90% of patients treated according to the disclosure ranges from 6-14 ng/mL. In embodiments, Day 2 Cₘₐₓ of brexpiprazole ranges from 10-22 ng/mL. In embodiments, Day 3 Cₘₐₓ of brexpiprazole ranges from 12-28 ng/mL. In embodiments, Day 4 Cₘₐₓ of brexpiprazole ranges from 13-34 ng/mL. In embodiments, Day 5 Cₘₐₓ of brexpiprazole ranges from 14-38 ng/mL. In embodiments, Day 6 Cₘₐₓ of brexpiprazole ranges from 15-41 ng/mL. In embodiments, Day 7 Cₘₐₓ of brexpiprazole ranges from 15-45 ng/mL. In embodiments, Day 8 Cₘₐₓ of brexpiprazole ranges from 22-59 ng/mL. In embodiments, Day 9 Cₘₐₓ of brexpiprazole ranges from 25-70 ng/mL. In embodiments, Day 10 Cₘₐₓ of brexpiprazole ranges from 28-79 ng/mL. In embodiments, Day 11 Cₘₐₓ of brexpiprazole ranges from 29-88 ng/mL. In embodiments, Day 12 Cₘₐₓ of brexpiprazole ranges from 30-95 ng/mL. In embodiments, Day 13 Cₘₐₓ of brexpiprazole ranges from 30-102 ng/mL. In embodiments, Day 14 Cₘₐₓ of brexpiprazole ranges from 30-107 ng/mL. In embodiments, Day 15 Cₘₐₓ of brexpiprazole ranges from 30-112 ng/mL. In embodiments, Day 16 Cₘₐₓ of brexpiprazole ranges from 30-116 ng/mL. In embodiments, Day 17 Cₘₐₓ of brexpiprazole ranges from 30-119 ng/mL. In embodiments, Day 18 Cₘₐₓ of brexpiprazole ranges from 30-122 ng/mL. In embodiments, Day 19 Cₘₐₓ of brexpiprazole ranges from 30-125 ng/mL. In embodiments, Day 20 Cₘₐₓ of brexpiprazole ranges from 30-128 ng/mL. In embodiments, Day 21 Cₘₐₓ of brexpiprazole ranges from 30-130 ng/mL. In embodiments, Day 22 Cₘₐₓ of brexpiprazole ranges from 30-132 ng/mL. In embodiments, Day 23 Cₘₐₓ of brexpiprazole ranges from 30-134 ng/mL. In embodiments, Day 24 Cₘₐₓ of brexpiprazole ranges from 30-136 ng/mL. In embodiments, Day 25 Cₘₐₓ of brexpiprazole ranges from 30-139 ng/mL. In embodiments, Day 26 v of brexpiprazole ranges from 30-141 ng/mL. In embodiments, Day 27 Cₘₐₓ of brexpiprazole ranges from 30-141 ng/mL. In embodiments, Day 28 Cₘₐₓ of brexpiprazole ranges from 30-142 ng/mL. In embodiments, the blood plasma concentrations of brexpiprazole range from about 80% to about 125% of the above values.

In embodiments, the Day 1 Cram of brexpiprazole for 90% of patients treated according to the disclosure ranges from 0-0 ng/mL. In embodiments, Day 2 Cₘᵢₙ of brexpiprazole ranges from 4-9 ng/mL. In embodiments, Day 3 Cₘᵢₙ of brexpiprazole ranges from 6-16 ng/mL. In embodiments, Day 4 Cₘᵢₙ of brexpiprazole ranges from 7-22 ng/mL. In embodiments, Day 5 Cₘᵢₙ of brexpiprazole ranges from 7-27 ng/mL. In embodiments, Day 6 Cₘᵢₙ of brexpiprazole ranges from 7-32 ng/mL. In embodiments, Day 7 Cₘᵢₙ of brexpiprazole ranges from 7-35 ng/mL. In embodiments, Day 8 Cₘᵢₙ of brexpiprazole ranges from 8-39 ng/mL. In embodiments, Day 9 Cₘᵢₙ of brexpiprazole ranges from 11-50 ng/mL. In embodiments, Day 10 Cₘᵢₙ of brexpiprazole ranges from 13-60 ng/mL. In embodiments, Day 11 Cₘᵢₙ of brexpiprazole ranges from 14-68 ng/mL. In embodiments, Day 12 Cₘᵢₙ of brexpiprazole ranges from 15-75 ng/mL. In embodiments, Day 13 Cₘᵢₙ of brexpiprazole ranges from 15-81 ng/mL. In embodiments, Day 14 Cₘᵢₙ of brexpiprazole ranges from 15-87 ng/mL. In embodiments, Day 15 Cₘᵢₙ of brexpiprazole ranges from 15-91 ng/mL. In embodiments, Day 16 Cₘᵢₙ of brexpiprazole ranges from 15-96 ng/mL. In embodiments, Day 17 Cₘᵢₙ of brexpiprazole ranges from 15-100 ng/mL. In embodiments, Day 18 Cₘᵢₙ of brexpiprazole ranges from 15-104 ng/mL. In embodiments, Day 19 Cₘᵢₙ of brexpiprazole ranges from 15-108 ng/mL. In embodiments, Day 20 Cₘᵢₙ of brexpiprazole ranges from 15-111 ng/mL. In embodiments, Day 21 Cₘᵢₙ of brexpiprazole ranges from 15-113 ng/mL. In embodiments, Day 22 Cₘᵢₙ of brexpiprazole ranges from 15-115 ng/mL. In embodiments, Day 23 Cₘᵢₙ of brexpiprazole ranges from 15-117 ng/mL. In embodiments, Day 24 Cₘᵢₙ of brexpiprazole ranges from 15-118 ng/mL. In embodiments, Day 25 Cₘᵢₙ of brexpiprazole ranges from 15-121 ng/mL. In embodiments, Day 26 Cₘᵢₙ of brexpiprazole ranges from 15-123 ng/mL. In embodiments, Day 27 Cₘᵢₙ of brexpiprazole ranges from 15-124 ng/mL. In embodiments, Day 28 Cₘᵢₙ of brexpiprazole ranges from 15-125 ng/mL. In embodiments, the blood plasma concentrations of brexpiprazole range from about 80% to about 125% of the above values.

In embodiments, the AUC₂₄ of brexpiprazole on Days 1-7 ranges from 108-972 ng*h/mL. In embodiments, the AUC₂₄ of brexpiprazole on Days 8-14 ranges from 380-2360 ng*h/mL. In embodiments, the AUC₂₄ of brexpiprazole on Days 15-21 ranges from 520-2917 ng*h/mL. In embodiments, the AUC₂₄ of brexpiprazole on Days 22-28 ranges from 522-3207 ng*h/mL.

In embodiments, the Cₘₐₓ of brexpiprazole on Days 1-7 ranges from 6-45 ng/mL. In embodiments, the Cₘₐₓ of brexpiprazole on Days 8-14 ranges from 22-107 ng/mL. In embodiments, the Cₘₐₓ of brexpiprazole on Days 15-21 ranges from 30-130 ng/mL. In embodiments, the Cₘₐₓ of brexpiprazole on Days 22-28 ranges from 30-142 ng/mL. In embodiments, the blood plasma concentrations of brexpiprazole range from about 80% to about 125% of the above values.

In embodiments, the Cₘᵢₙ of brexpiprazole on Days 1-7 ranges from 0-35 ng/mL. In embodiments, the Cₘᵢₙ of brexpiprazole on Days 8-14 ranges from 8-87 ng/mL. In embodiments, the Cₘᵢₙ of brexpiprazole on Days 15-21 ranges from 15-113 ng/mL. In embodiments, the Cₘᵢₙ of brexpiprazole on Days 22-28 ranges from 15-125 ng/mL. In embodiments, the blood plasma concentrations of brexpiprazole range from about 80% to about 125% of the above values.

Pharmacokinetics Achieved at Steady State

In some embodiments, the modified dosage regimens described herein provide therapeutically effective levels of brexpiprazole for certain patient populations (e.g., for patients that are obese or are obese CYP2D6 poor metabolizers).

In some embodiments, after administering between about 0.5 mg and about 8 mg (e.g., 0.25 mg, 0.5 mg, 1.0 mg, 2.0 mg, 2.5 mg, 3.0 mg, 3.5 mg, 4.0 mg, 5.0 mg, 6.0 mg, 7.5 mg, 8 mg) of brexpiprazole, the obese CYP2D6 PM or obese CYP2D6 EM patient treated according to the modified dosing regimens disclosed herein have a steady state minimum observed plasma drug concentration (C_{min, ss}) between about 30 ng/mL and about 120 ng/mL. In some embodiments, the C_{min, ss} is between about 30 ng/mL and about 60 ng/mL nine days after administration of the day 1 dose of brexpiprazole. In some embodiments, the C_{min, ss} is between about 60 ng/mL and about 95 ng/mL 16 days after administration of the day 1 dose of brexpiprazole. In some embodiments, the C_{min, ss} is at least about 40.4 ng/mL. In some embodiments, the C_{min, ss} is at least about 90.9 ng/mL. In some embodiments, the C_{min, ss} is at least about 10.1 ng/mL. In some embodiments, the C_{min, ss} is at least about 40.4 ng/mL on day 14 of brexpiprazole administration. In some embodiments, the C_{min, ss} is at least about 90.9 ng/mL on day 14 of brexpiprazole administration. In some embodiments, the C_{min, ss} is at least about 10.1 ng/mL on day 14 of brexpiprazole administration. In some embodiments, the C_{min, ss} is about 30 ng/mL, about 35 ng/mL, about 40 ng/mL, about 45 ng/mL, about 50 ng/mL, about 55 ng/mL, about 60 ng/mL, about 65 mg/mL, about 70 ng/mL, about 75 ng/mL, about 80 ng/mL, about 85 ng/mL, about 90 ng/mL, about 95 ng/mL, about 100 ng/mL, about 105 ng/mL, about 110 ng/mL, about 115 ng/mL, about 120 ng/mL, about 125 ng/mL, or about 130 ng/mL, including all ranges and values in between. In some embodiments, the Cₘᵢₙ is between 80% and 125% of any of the aforementioned values or ranges between the aforementioned values.

In some embodiments, the time to reach C_{min, ss} is reduced after dosage of brexpiprazole according to a modified dosage regimen described herein as compared to dosage according to the brexpiprazole (REXULTI®) FDA label dated March 2020. Example 2 shows that the time to reach therapeutic concentrations of brexpiprazole using the modified dosage regimen is reduced in the patient populations described herein as compared to the time to reach C_{min, ss} using the brexpiprazole (REXULTI®) FDA Label. In some embodiments, the time to reach Cₘᵢₙ according to a modified dosage regimen is reduced by between about 1 day and about 35 days, for example, at least about 1 day, 2 days, 3 days, 4 days, 5 days, 6 days, 7 days, 8 days, 9 days, days, 11 days, 12 days, 13 days, 14 days, 15 days, 16 days, 17 days, 18 days, 19 days, 2 days, 21 days, 22 days, 23 days, 24 days, 25 days, 26 days, 27 days, 28 days, 29 days, 30 days, 31 days, 32 days, 33 days, 34 days, or at least about 35 days, including all values and ranges there between as compared to dosage according to the brexpiprazole (REXULTI®) FDA label.

In some embodiments, after administering between about 0.5 mg and about 8 mg (e.g., 0.25 mg, 0.5 mg, 1.0 mg, 2.0 mg, 2.5 mg, 3.0 mg, 3.5 mg, 4.0 mg, 5.0 mg, 6.0 mg, 7.5 mg, 8 mg) of brexpiprazole, the obese CYP2D6 PM patient or obese CYP2D6 EM patient has a steady state maximum observed plasma drug concentration (C_{max, ss} is between about 50 ng/mL and about 150 ng/mL. In some embodiments, the C_{max, ss} is between about 50 ng/mL and about 100 ng/mL nine days after administration of the day 1 dose of brexpiprazole. In some embodiments, the Cₘₐₓ, SS is between about 80 ng/mL and about 150 ng/mL 16 days after administration of the day 1 dose of brexpiprazole. In some embodiments, the C_{max, ss} is about 50 ng/mL, about 55 ng/mL, about ng/mL, about 65 mg/mL, about 70 ng/mL, about 75 ng/mL, about 80 ng/mL, about 85 ng/mL, about 90 ng/mL, about 95 ng/mL, about 100 ng/mL, about 105 ng/mL, about 110 ng/mL, about 115 ng/mL, about 120 ng/mL, about 125 ng/mL, about 130 ng/mL, about 135 ng/mL, about 140 ng/mL, about 145 ng/mL, or about 150 ng/mL including all ranges and values in between. In some embodiments, the Cₘₐₓ is between 80% and 125% of any of the aforementioned values or ranges between the aforementioned values.

In some embodiments, after administering between about 0.25 mg and about 8 mg (e.g., 0.25 mg, 0.5 mg, 1.0 mg, 2.0 mg, 2.5 mg, 3.0 mg, 3.5 mg, 4.0 mg, 5.0 mg, 6.0 mg, 7.5 mg, 8 mg,) of brexpiprazole, the obese CYP2D6 PM or obese CYP2D6 EM patient has a steady state area under the concentration time curve from day 0 to day 9 (AUC_{9, ss}) between 1000 ng*hr/mL and 2000 ng*hr/mL. In some embodiments, after administering between about 0.5 mg and about mg, the patient has an AUC_{9, ss} of between 1500 ng*hr/mL and 2000 ng*hr/mL. In some embodiments, the AUC_{9, ss} is about 1000 ng*hr/mL, about 1100 ng*hr/mL, about 1200 ng*hr/mL, about 1300 ng*hr/mL, about 1400 ng*hr/mL, about 1500 ng*hr/mL, about 1600 ng*hr/mL, about 1700 ng*hr/mL, about 1800 ng*hr/mL, about 1900 ng*hr/mL, or about 2000 ng*hr/mL, including all ranges and values in between. In some embodiments, the AUC_{9, ss} is between % and 125% of the aforementioned values.

In some embodiments, after administering between about 0.25 mg and about 8 mg (e.g., 0.25 mg, 0.5 mg, 1.0 mg, 2.0 mg, 2.5 mg, 3.0 mg, 3.5 mg, 4.0 mg, 5.0 mg, 6.0 mg, 7.5 mg, 8 mg) of brexpiprazole, the obese CYP2D6 PM patient or obese CYP2D6 EM patient has a steady state area under the concentration time curve from day 0 to day 16 (AUC_{16, ss}) between 1800 ng*hr/mL and 2600 ng*hr/mL. In some embodiments, after administering between about mg and about 10 mg, the patient has an AUC_{16, ss} between 2000 ng*hr/mL and 2600 ng*hr/mL. In some embodiments, after administering between about 0.5 mg and about 10 mg, the patient has an AUC_{16, ss} between 2000 ng*hr/mL and 2300 ng*hr/mL. In some embodiments, the AUC_{16, ss} is about 1800 ng*hr/mL, about 1900 ng*hr/mL, about 2000 ng*hr/mL, about 2100 ng*hr/mL, about 2200 ng*hr/mL, about 2300 ng*hr/mL, about 2400 ng*hr/mL, about 2500 ng*hr/mL, or about 2600 ng*hr/mL, including all ranges and values in between. In some embodiments, the AUC_{16, ss} is between 80% and 125% of the aforementioned values.

All documents or patents cited herein are incorporated by reference in their entireties for all purposes.

The following examples are offered by way of illustration and not by way of limitation.

### Example 1. Brexpiprazole Pharmacokinetics in Obese and Obese CYP2D6 PM

Brexpiprazole is an atypical antipsychotic indicated to treat schizophrenia and for use as an adjunctive therapy to antidepressants and for the treatment of major depressive disorder. Brexpiprazole is known to have a drug-drug interaction with CYP2D6 inhibitors, and dose reductions are recommended for patients that are cytochrome P450 CYP2D6 poor metabolizers (PM) or patients taking concomitant CYP2D6 inhibitors. CYP2D6 extensive metabolizer (EM) patients metabolize brexpiprazole normally, whereas CYP2D6 PM patients are believed to eliminate brexpiprazole more slowly. The FDA label of brexpiprazole (REXULTI®) advises that these patient populations take half of the usual dose of brexpiprazole. Table A shows the recommended dosing schedule of brexpiprazole for treating schizophrenia in patients that are CYP2D6 PM and CYP2D6 EM. The brexpiprazole FDA label does not contain recommendations for brexpiprazole dosage according to body size (obesity status). Therefore, obese patients (as described herein) are treated according to the same dosing schedule as EM or PM (depending on their CYP2D6 metabolizer status)

TABLE A
Brexpiprazole Dosing for Schizophrenia According to FDA Label

| CYP2D6 status | Days 1-4 | Days 5-7 | Days 8+ |
|---|---|---|---|
| EM | 1 mg QD | 2 mg QD | 2-4 mg QD |
| PM | 0.5 mg QD | 1 mg QD | 1-2 mg QD |

PBPK Modeling of Patients Dosed with Brexpiprazole According to the FDA Label

Physiologically based pharmacokinetic (PBPK) modeling was used to estimate the pharmacokinetic parameters of brexpiprazole of the following schizophrenia patient populations after administering brexpiprazole according to the FDA label (REXULTI® label dated March 2020): obese (BMI>35 kg/m 2), normal weight (BMI=18 kg/m 2-25 kg/m 2), CYP2D6 poor metabolizer (PM), and CYP2D6 extensive metabolizer (EM). Although the PBPK modeling was based on the dosing regimen for schizophrenia according to the FDA label (REXULTI®, March 2020), pharmacokinetic parameters are dose-dependent and expected to be similar in MDD.

In order to simulate brexpiprazole under various conditions, a whole-body PBPK model was constructed to capture the drug's kinetics in major tissues. Model tissue compartments included adipose, bone, brain, large intestine, small intestine, heart, kidney, liver, lung, muscle, spleen, stomach, and skin tissues, as well as venous and arterial blood compartments. In order to accurately capture first-pass clearance effects on the drug, the model also included correct representation of the gastrointestinal tract organs and the liver. Additionally, a peripheral sampling site compartment was used to represent the PK-sampled venous blood concentration as it was found to more accurately capture referenced plasma concentrations. Drug distribution into each tissue compartment was modeled assuming perfusion-limited kinetics, with partitioning into tissue described by a partition coefficient (Kp) estimated using methods described by Poulin and Theil (2002). Brexpiprazole-specific biochemical parameters such as the log of octanol:water partition coefficient (log P), negative log acid dissociation constant (pKa), fraction unbound in the plasma (fᵤₚ), blood:plasma concentration ratio (BP), and clearance were obtained from literature; additional physiochemical properties used to calculate Kps were obtained via DrugBank. Absorption was modeled assuming simple first-order absorption, and clearance of brexpiprazole was assumed to occur entirely in the liver (˜1% renal clearance). Based on the literature it was assumed that 46.7% of brexpiprazole clearance is due to CYP3A4, with the remaining clearance due to CYP2D6 (43.3%) and other routes (10%). The impact of CYP2D6 PM status is known to decrease baseline clearance by ˜30%, which was reflected in the reduced baseline clearance parameters due to CYP2D6 in PM simulations. Simulations were carried out in a virtual population of 500 normal-weight and 500 obese individuals with age- and sex-specific physiological parameters (i.e. tissue flows and volumes) sampled from the National Health and Nutrition Examination Survey (NHANES) dataset.

Model parameters were calibrated and subsequently qualified by digitizing PK data from available literature and comparing predicted vs. observed AUC_{0-∞}, Cₘₐₓ, and time to Cₘₐₓ (Tₘₐₓ) under various dose strengths, dosing scenarios (i.e., single dose vs. multiple dose), and routes of administration (i.e., intravenous or oral formulations). Calibration and qualification simulations were carried out using a single typical individual (male, age=30 years, weight=73 kg, and height=1.76 m). All comparisons yielded a geometric mean fold error less than or equal to 2, and thus were considered accurate for these purposes.

The Applicant's model was validated and shown to accurately predict observed and literature pharmacokinetic parameters as shown in Tables B and C, below.

TABLE B
Model Validation

| | | | AUC (ng.h/mL) | | | Cmax (ng/mL) | | | Tmax (h) | | | |
|---|---|---|---|---|---|---|---|---|---|---|---|---|
| Reference | Route | Dose (mg) | Observed | Predicted | GMFE | Observed | Predicted | GMFE | Observed | Predicted | GMFE | Calibration |
| NDA | IV | 0.25 | 172 | 175 | 1.02 | 4.73 | 5.07 | 1.07 | 1 | 1 | 1.0 | Yes |
| Study 1 | PO | 2 | 1690 | 1350 | 1.25 | 22.10 | 19.5 | 1.13 | 6 | 4 | 1.5 | Yes |
| | PO | 0.2 | 123 | 135 | 1.10 | 2.62 | 1.95 | 1.35 | 2 | 4 | 2.0 | No |
| | PO | 0.5 | 287 | 337 | 1.17 | 6.64 | 4.87 | 1.36 | 4 | 4 | 1.0 | No |
| | PO | 1 | 636 | 675 | 1.06 | 11.70 | 9.74 | 1.20 | 6 | 4 | 1.5 | No |
| NDA | PO | 2 | 2160 | 1350 | 1.60 | 24.60 | 19.50 | 1.27 | 4 | 4 | 1.0 | No |
| Study 2 | PO | 4 | 2760 | 2700 | 2.00 | 47.20 | 38.90 | 1.21 | 6 | 4 | 1.5 | No |
| | PO | 6 | 5230 | 4050 | 1.29 | 71.10 | 58.40 | 1.22 | 6 | 4 | 1.5 | No |
| | PO | 8 | 7920 | 5400 | 1.47 | 79.20 | 77.90 | 1.02 | 6 | 4 | 1.5 | No |

GMFE = geometric mean fold error
Calibration: Data used to optimize and/or refine PK parameters in the final model
References are from NDA 205422 Clinical Pharmacology and Biopharmaceutics Review, p. 26 and 28

TABLE C
Comparison to Literature CYP2D6 PM PK Values
Brexpiprazole Model Comparisons

| | | AUC | | AUC GMR | | | % Expected GMR | |
|---|---|---|---|---|---|---|---|---|
| Cohort | Dose | Normal | Obese | Expected | Normal | Obese | Normal | Obese |
| CYP 2D6 EM | 4 mg | 2703 | 2779 | 1.0 | 1.00 | 1.00 | 100% | 100% |
| CYP 2D6 PM | 4 mg | 4289 | 4397 | 1.5 | 1.59 | 1.58 | 106% | 105% |
| CYP 2D6 PM | 2 mg | 2134 | 2093 | 0.75 | 0.79 | 0.75 | 105% | 100% |

″Expected″ values are based on population PK analysis in NDA 205422 Clinical Pharmacology and Biopharmaceutics Review

The modeling data showed that patients that are obese CYP2D6 EM and obese CYP2D6 PM take significantly longer to reach therapeutic plasma levels of brexpiprazole when initiating brexpiprazole treatment (Table D). This is based, at least in part, on Applicant's surprising discovery that the half-life of brexpiprazole is dependent on both body size and CYP2D6 metabolizer status. Normal-weight CYP2D6 EM exhibit a lower mean half-life than patients that are obese CYP2D6 EM or obese CYP2D6 PM (Table D).

TABLE D
Half-Life of Brexpiprazole in Different Patient Poplulations

| Weight | CYP2D6 | Mean half-life (SD), hours | |
|---|---|---|---|
| Normal | EM | 68.6 | (45.0) |
| Obese | EM | 192 | (130) |
| Normal | PM | 107 | (71.3) |
| Obese | PM | 297 | (207) |

Additionally, obese CYP2D6 EM and obese CYP2D6 PM patients take longer to reach steady state plasma levels and therapeutic concentrations compared to normal weight CYP2D6 EM when dosed using the instructions on the FDA label (Table E where EC50, EC80, and EC90 correspond to the plasma concentration at with 50%, 80%, or 90% of the population is expected to respond to treatment, respectively). Consequently, these patients take significantly longer to be treated than previously known, and some patients may not actually reach therapeutic brexpiprazole concentrations. Thus, obese CYP2D6 EM and obese CYP2D6 PM schizophrenia patients dosed according to the FDA label (e.g., REXULTI® label dated December 2021) are not effectively treated. Ineffective schizophrenia treatment results in severe complications, including suicide attempts, anxiety, depression, alcohol or drug abuse, inability to work or attend school, financial problems, homelessness, social isolation, health and medical problems, being victimized, and aggressive behavior.

TABLE E
Pharmacokinetics Based on Current FDA Label

| | | Days to Reach Pharmacokinetic | | |
|---|---|---|---|---|
| | | Endpoints | | |
| Weight | CYP2D6 | EC50 | EC80 | Steady State |
| Normal | EM | 3 | 9 | 21 |
| Obese | EM | 6 | 11 | 37 |
| Normal | PM | 5 | 10 | 24 |
| Obese | PM | 7 | 15 | 46 |

### Example 2. Schizophrenia

PBPK Modeling of Schizophrenia Patients Treated with Higher Doses According to the Disclosure

Modified dosing regimens were developed so that obese CYP2D6 EM, obese CYP2D6 PM, and normal-weight CYP2D6 PM patients reach therapeutic levels earlier (Table F). The modified dosage regimens comprises increasing the total daily dose of brexpiprazole for at least the first 15 days of treatment. Thereafter (e.g., on day 16), patients returned to administering the recommended dose once daily.

TABLE F
Modified Schizophrenia Dosing Regimens

| | CYP2D6 | | | | |
|---|---|---|---|---|---|
| Weight | Status | Days 1-4 | Days 5-7 | Days 8-14 | Days 15+ |
| All (Label) | EM | 1 mg QD | 2 mg QD | 4 mg QD | 4 mg QD |
| Obese (Modified | EM | 1.5 mg QD | 3 mg QD | 6 mg QD | 4 mg QD |
| Dosing Regimen 1.1) | | | | | |
| Obese (Modified | EM | 1.25 mg QD | 2.5 mg QD | 5 mg QD | 4 mg QD |
| Dosing Regimen 2.1) | | | | | |
| All (Label) | PM | 0.5 mg QD | 1 mg QD | 2 mg QD | 2 mg QD |
| Obese (Modified | PM | 1.25 mg BID | 2.5 mg BID | 5 mg BID | 2 mg QD |
| Dosing Regimen 3.1) | | | | | |
| Obese (Modified | PM | 1.5 mg BID | 3 mg BID | 6 mg BID | 2 mg QD |
| Dosing Regimen 4.1) | | | | | |
| All (Label) | PM | 0.5 mg QD | 1 mg QD | 2 mg QD | 2 mg QD |
| Normal-weight | PM | 0.75 mg QD | 1.5 mg QD | 3 mg QD | 2 mg QD |
| (Modified | | | | | |
| Dosing Regimen 9.1) | | | | | |

*Bold Text indicates differences from instructions on FDA label for brexpiprazole (REXULTI ®)

PBPK modeling shows that the pharmacokinetic parameters of obese CYP2D6 PM and obese CYP2D6 EM schizophrenia patients treated with the modified dosage regimen of Table F approach those of normal-weight CYP2D6 EM.

FIGS. 1B, 1D, and 1F shows that obese CYP2D6 PM schizophrenia patients that are treated according to Modified Dosing Regimen 3.1 and 4.1 have plasma brexpiprazole concentrations that are similar to normal-weight CYP2D6 EM patients. Obese CYP2D6 PM patients that are administered 125% or 150% of the usual starting and recommended daily doses for CYP2D6 EM the first 15 days reach therapeutic concentrations faster than obese CYP2D6 PM patients that are administered brexpiprazole according to the FDA label.

FIGS. 2A-2C show that obese CYP2D6 EM schizophrenia patients that are treated according to Modified Dosing Regimen 1.1 and 1.2 have plasma brexpiprazole concentrations that are similar to normal-weight CYP2D6 EM patients. Obese CYP2D6 PM patients that are administered 125% or 150% of the usual starting and recommended daily doses for the first 15 days reach therapeutic concentrations faster than obese CYP2D6 EM patients that are administered brexpiprazole according to the FDA label.

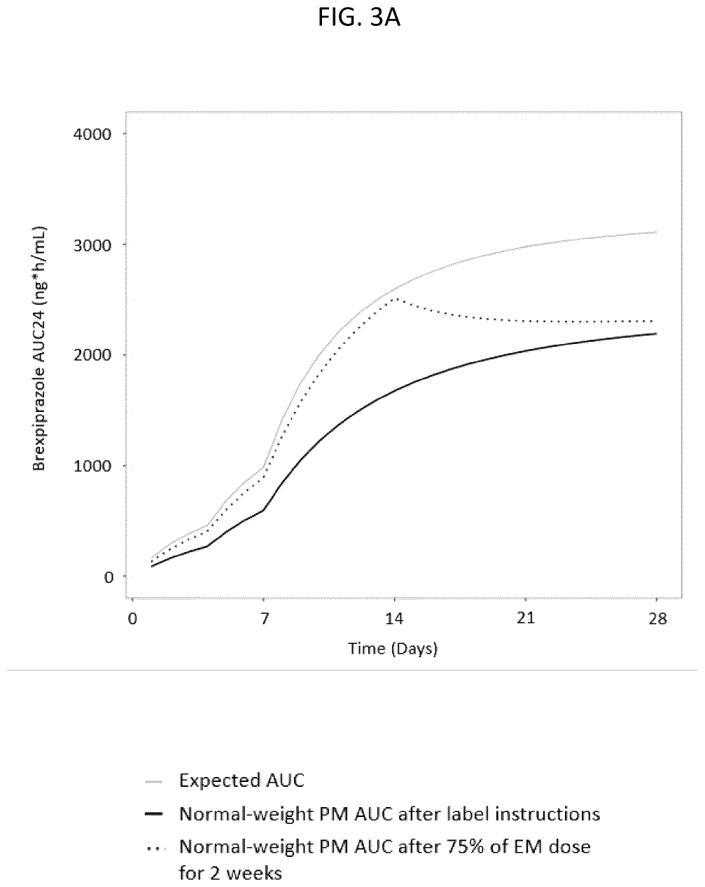

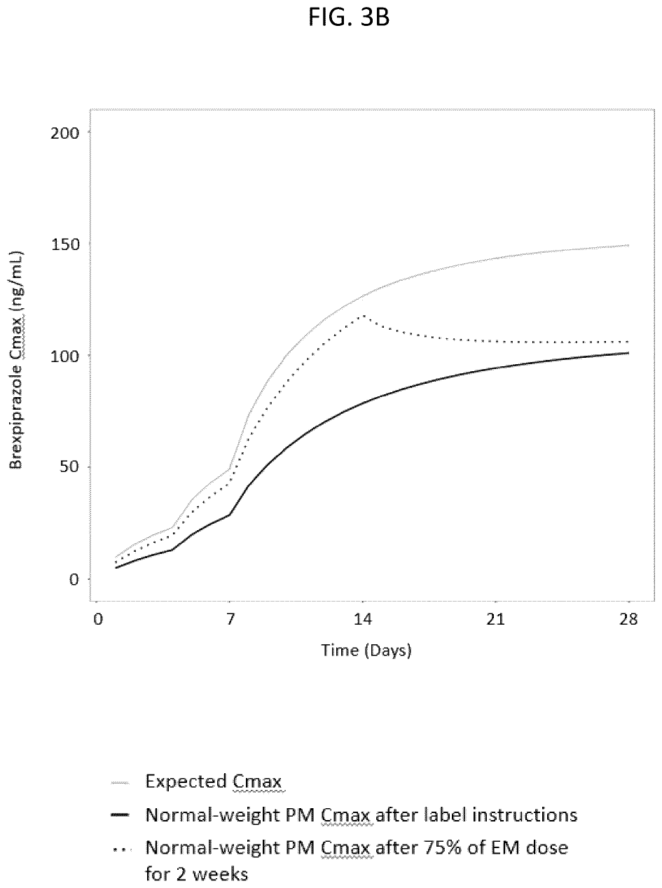

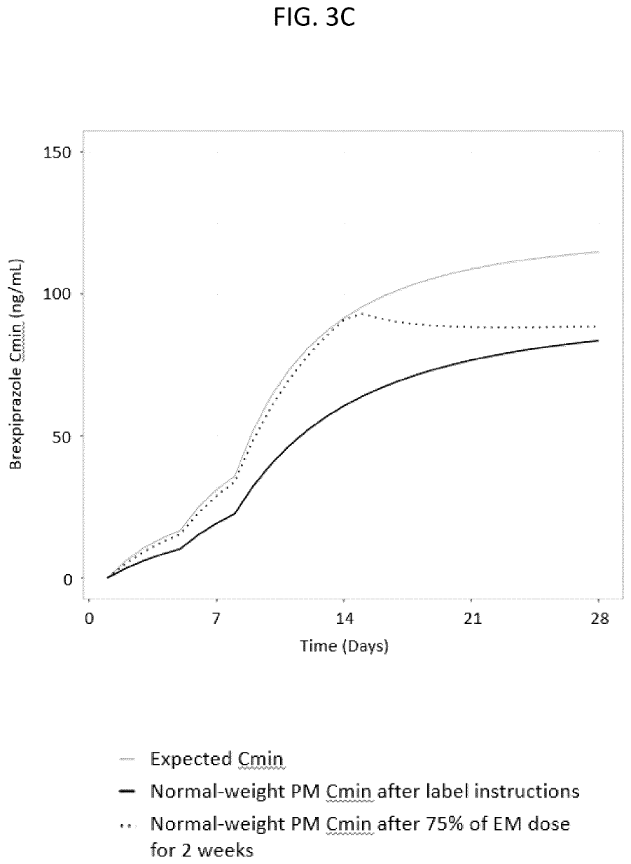

FIGS. 3A-3C show that normal-weight CYP2D6 PM schizophrenia patients that are treated according to Modified Dosing Regimen 9.1 have plasma brexpiprazole concentrations that are similar to normal-weight CYP2D6 EM patients. Normal-weight CYP2D6 PM patients that are administered 75% of the usual starting and recommended daily doses for the first 15 days reach therapeutic concentrations faster than normal-weight CYP2D6 EM patients that are administered 50% of the usual doses of brexpiprazole according to the FDA label.

### Example 3. Major Depressive Disorder (Mdd)

PBPK Modeling of MDD Patients Treated with Higher Doses According to Modified Methods

A modified dosing regimen was developed so that obese and CYP2D6 PM patients reach therapeutic levels earlier (Table G). For obese CYP2D6 EM, the modified dosage regimen comprises doubling the total daily dose of brexpiprazole for days at least the first 14 or 21 days of the dosage regimen on the brexpiprazole label. Thereafter, (e.g., on day 15 or day 22), the patients are treated with the recommended dose once daily according to the brexpiprazole label.

TABLE G
Modified MDD Dosing Regimens

| | CYP2D6 | | | | |
|---|---|---|---|---|---|
| Weight | Status | Days 1-7 | Days 8-14 | Days 15-21 | Days 21+ |
| All (Label) | EM | 0.5 mg QD | 1 mg QD | 2 mg QD | 2-3 mg QD |
| Obese (Modified | EM | 0.75 mg QD | 1.5 mg QD | 2-3 mg QD | 2-3 mg QD |
| Dosing Regimen A.1) | | | | | |
| Obese (Modified | EM | 0.625 mg QD | 1.25 mg QD | 2.5 mg QD | 2-3 mg QD |
| Dosing Regimen B.1) | | | | | |
| All (Label) | EM | 1.0 mg QD | 2 mg QD | 2-3 mg QD | 2-3 mg QD |
| Obese (Modified | EM | 1.5 mg QD | 3 mg QD | 2-3 mg QD | 2-3 mg QD |
| Dosing Regimen C.1) | | | | | |
| Obese (Modified | EM | 1.25 mg QD | 2.5 mg QD | 2.5 mg QD | 2-3 mg QD |
| Dosing Regimen D.2) | | | | | |
| All (Label) | PM | 0.25 mg QD | 0.5 mg QD | 1.0 mg QD | 1-1.5 mg |
| Obese (Modified | PM | 0.75 mg QD | 1.5 mg QD | 1-1.5 mg QD | 1-1.5 mg QD |
| Dosing Regimen E.1) | | | | | |
| Obese (Modified | PM | 0.625 mg QD | 1.25 mg QD | 2.5 mg QD | 1-1.5 mg QD |
| Dosing Regimen F.1) | | | | | |
| All (Label) | PM | 0.5 mg QD | 1 mg QD | 1-1.5 mg QD | 1-1.5 mg QD |
| Obese (Modified | PM | 1.5 mg QD | 3 mg QD | 1-1.5 mg QD | 1-1.5 mg QD |
| Dosing Regimen G.1) | | | | | |
| Obese (Modified | PM | 1.25 mg QD | 2.5 mg QD | 2.5 mg QD | 1-1.5 mg QD |
| Dosing Regimen H.1) | | | | | |
| All (Label) | PM | 0.25 mg QD | 0.5 mg QD | 1 mg QD | 1 mg QD |
| Normal-Weight | PM | 0.375 mg QD | 0.75 mg QD | 1.5 mg QD | 1 mg QD |
| (Modified | | | | | |
| Dosing Regimen Q.1) | | | | | |
| All (Label) | PM | 0.5 mg QD | 1 mg QD | 1 mg QD | 1 mg QD |
| Normal-Weight | PM | 0.75 mg QD | 1.5 mg QD | 3 mg QD | 1 mg QD |
| (Modified | | | | | |
| Dosing Regimen S.1) | | | | | |

PBPK modeling shows that the pharmacokinetic parameters of obese CYP2D6 PM and obese CYP2D6 EM schizophrenia patients treated with the modified dosage regimen of Table G approach those of normal-weight CYP2D6 EM.

FIGS. 4A-4C shows that obese CYP2D6 PM MDD patients that are treated according to the Modified Dosing Regimens E.1 and F.1 have plasma brexpiprazole concentrations that are similar to normal-weight CYP2D6 EM patients. Obese CYP2D6 PM patients that are administered 125% or 150% of the usual starting dose of 0.5 mg and recommended daily doses for CYP2D6 EM for the first 15 days reach therapeutic concentrations faster than obese CYP2D6 EM patients that are administered brexpiprazole according to the FDA label.

FIG. 5A-5C shows that obese CYP2D6 EM MDD patients that are treated according to the Modified Dosing Regimen A.1 and B.1 have plasma brexpiprazole concentrations that are similar to normal-weight CYP2D6 EM patients. Obese CYP2D6 EM patients that are administered 125% or 150% of the usual starting of 0.5 mg and recommended daily doses for CYP2D6 EM for the first 15 days reach therapeutic concentrations faster than obese CYP2D6 EM patients that are administered brexpiprazole according to the FDA label.

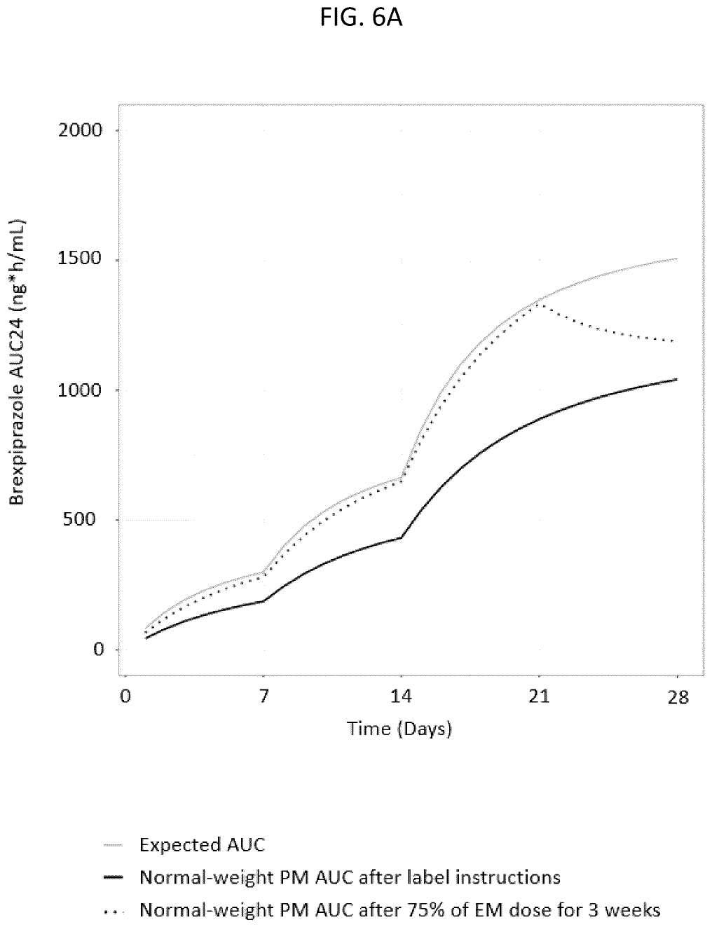

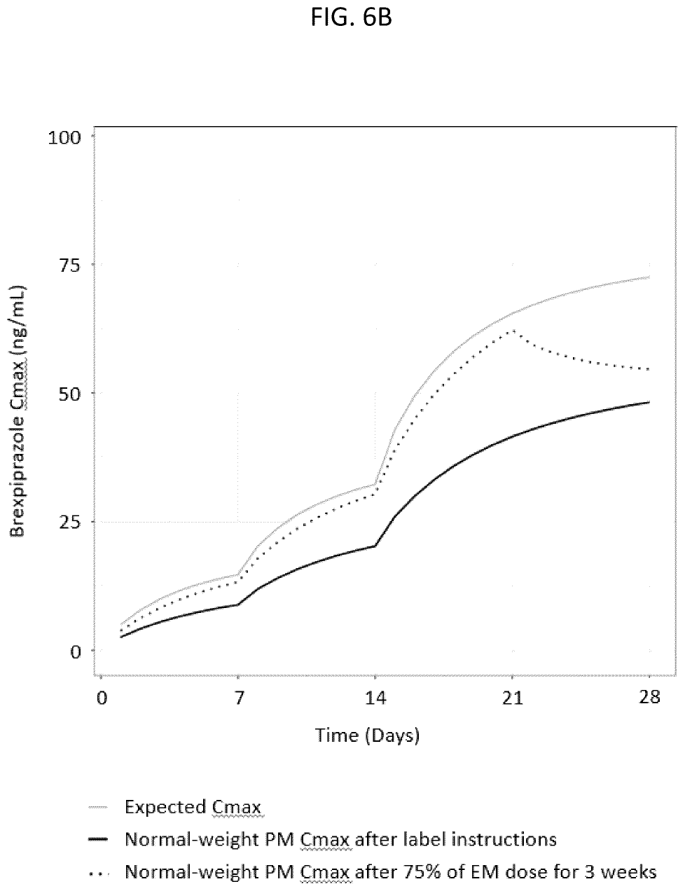

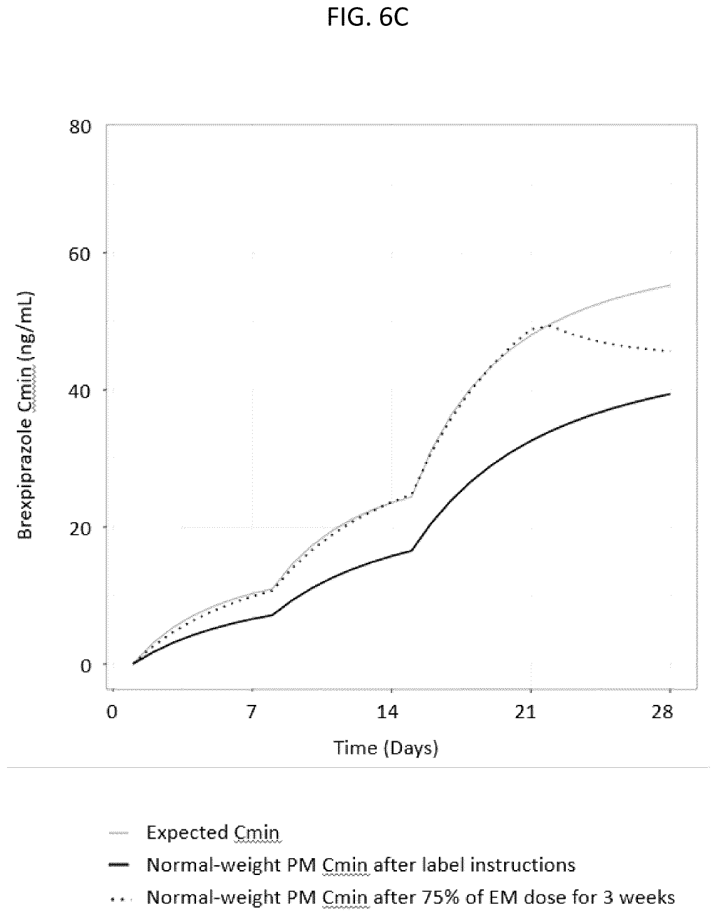

FIG. 6A-6C shows that normal-weight CYP2D6 PM MDD patients that are treated according to Modified Dosing Regimen Q.1 reach therapeutic concentrations in a similar time to normal-weight CYP2D6 EM patients. Normal-weight CYP2D6 PM patients that are administered 75% of the usual starting of 0.5 mg and recommended daily doses for the first 15 days reach therapeutic concentrations faster than normal-weight CYP2D6 EM patients that are administered 50% of the usual doses of brexpiprazole according to the FDA label

FIGS. 7A-7C shows that obese CYP2D6 PM MDD patients that are treated according to the Modified Dosing Regimen G.1 and H.1 have plasma brexpiprazole concentrations that are similar to normal-weight CYP2D6 EM patients. Obese CYP2D6 PM patients that are administered 125% or 150% of the usual starting dose of 1.0 mg and recommended daily doses for CYP2D6 EM for the first 15 days reach therapeutic concentrations faster than obese CYP2D6 EM patients that are administered brexpiprazole according to the FDA label.

FIGS. 8A-8C shows that obese CYP2D6 EM MDD patients that are treated according to the Modified Dosing Regimen C.1 and D.1 have plasma brexpiprazole concentrations that are similar to normal-weight CYP2D6 EM patients. Obese CYP2D6 EM patients that are administered 125% or 150% of the usual starting dose of 1.0 mg and recommended daily doses for CYP2D6 EM for the first 15 days reach therapeutic concentrations faster than obese CYP2D6 EM patients that are administered brexpiprazole according to the FDA label.

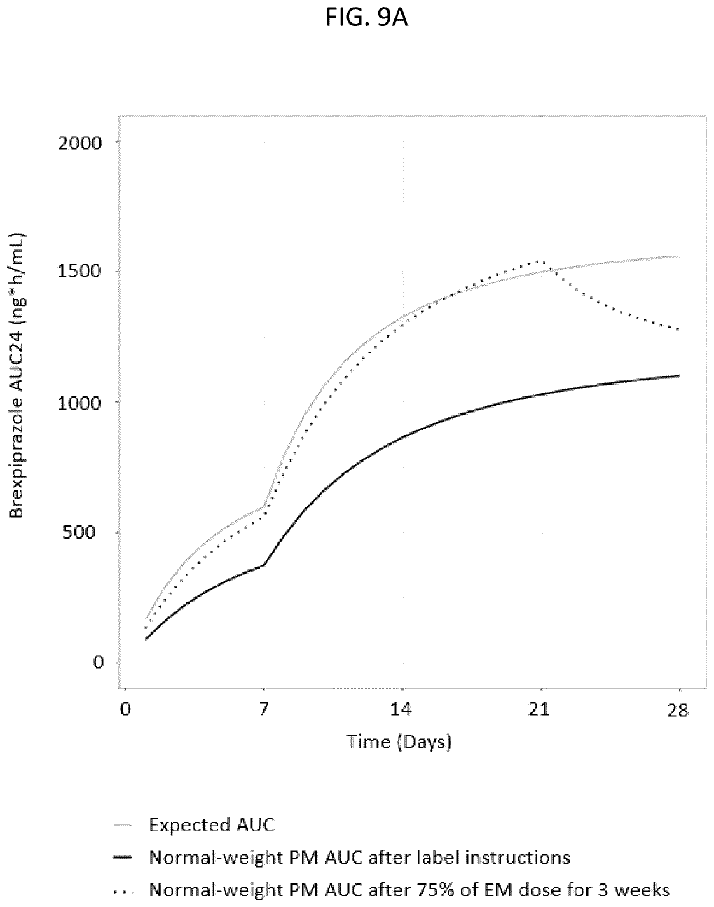

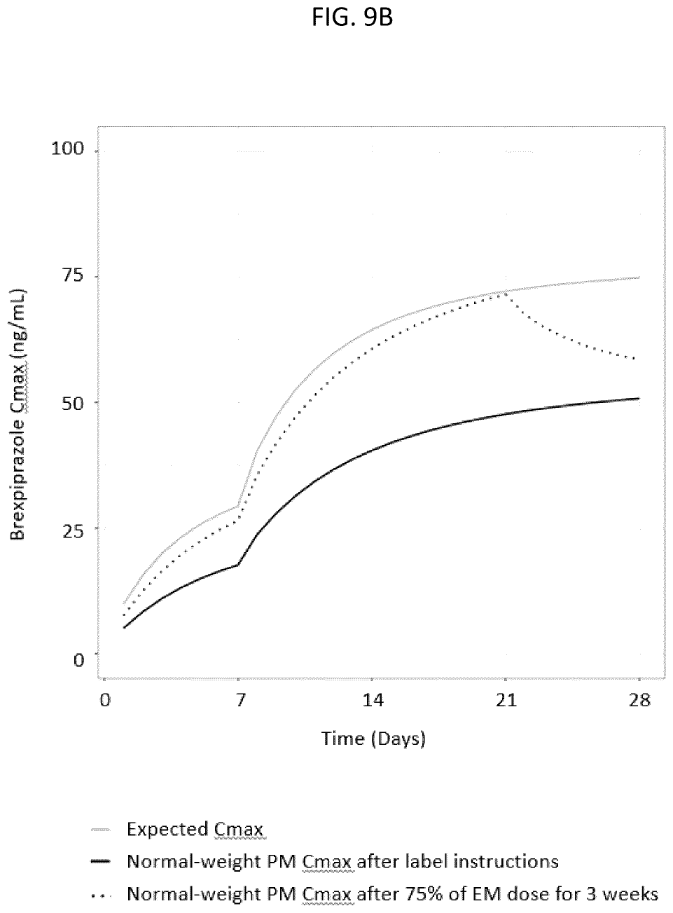

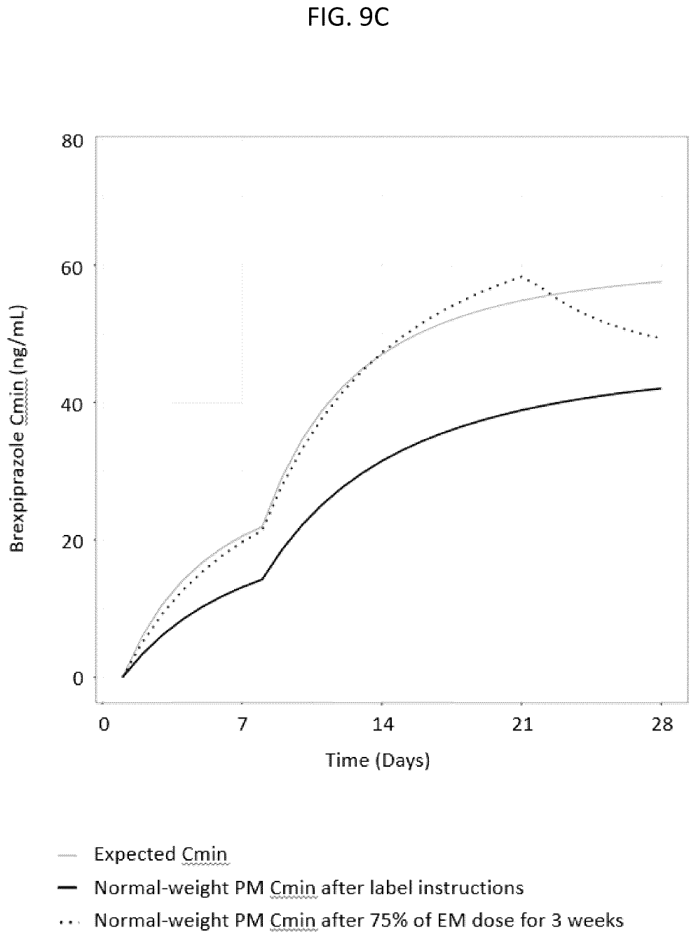

FIGS. 9A-9C shows that normal-weight CYP2D6 PM MDD patients that are treated according to the Modified Dosing Regimen S.1 have plasma brexpiprazole concentrations that are similar to normal-weight CYP2D6 EM patients. Normal-weight CYP2D6 PM patients that are administered 75% of the usual starting dose of 1.0 mg and recommended daily doses for CYP2D6 EM for the first 15 days reach therapeutic concentrations faster than obese CYP2D6 EM patients that are administered brexpiprazole according to the FDA label.

1. A method of initiating treatment of schizophrenia with brexpiprazole in an obese patient who is not a CYP2D6 poor metabolizer, comprising:
(a) orally administering 1.125-2 mg brexpiprazole once daily on each of the first 4 days of brexpiprazole treatment;
(b) orally administering 2.125-4 mg brexpiprazole once daily on each of the next 3 days following step (a);
(c) orally administering 4.125-6 mg brexpiprazole on each of the next 7 days following step (c); and then
(d) orally administering a recommended dose of 2-4 mg brexpiprazole once daily thereafter;
wherein the obese patient has one or more of the following characteristics:
(i) BMI of at least about 35;
(ii) % IBW of at least about 150%;
(iii) waist size greater than about 42 inches;
(iv) % body fat greater than about 40%;
(v) % android body fat greater than about 40%;
(vi) % gynoid body fat greater than about 40%; or
(vii) total body fat greater than about 40 kg.

2. The method of claim 1, wherein 1.25-2 mg of brexpiprazole is administered in step (a).

3. The method of claim 1, wherein 1.25 mg of brexpiprazole is administered in step (a).

4. The method of claim 1, wherein 1.5 mg of brexpiprazole is administered in step (a).

5. The method of claim 1, wherein 2 mg of brexpiprazole is administered in step (a).

6. The method of claim 1, wherein 2.25-3.75 mg of brexpiprazole is administered in step (b).

7. The method of claim 1, wherein 2.5 mg of brexpiprazole is administered in step (b).

8. The method of claim 1, wherein 3 mg of brexpiprazole is administered in step (b).

9. The method of claim 1, wherein 4.25-6 mg of brexpiprazole is administered in step (c).

10. The method of claim 1, wherein 6 mg of brexpiprazole is administered in step (c).

11. The method of claim 1, wherein 5 mg of brexpiprazole is administered in step (c).

12. The method of claim 1, wherein the recommended dose of brexpiprazole is 2 mg/day.

13. The method of claim 1, wherein the recommended dose of brexpiprazole is 2.25 mg/day.

14. The method of claim 1, wherein the recommended dose of brexpiprazole is 2.5 mg/day.

15. The method of claim 1, wherein the recommended dose of brexpiprazole is 2.75 mg/day.

16. The method of claim 1, wherein the recommended dose of brexpiprazole is 3 mg/day.

17. The method of claim 1, wherein the recommended dose of brexpiprazole is 3.25 mg/day.

18. The method of claim 1, wherein the recommended dose of brexpiprazole is 3.5 mg/day.

19. The method of claim 1, wherein the recommended dose of brexpiprazole is 3.75 mg/day.

20. The method of claim 1, wherein the recommended dose of brexpiprazole is 4 mg/day.

21. A method of initiating treatment of schizophrenia with brexpiprazole in an obese patient who is a CYP2D6 poor metabolizer, comprising:
(a) orally administering 1.125-2 mg brexpiprazole once daily on each of the first 4 days of brexpiprazole treatment;
(b) orally administering at least 2.125-3.875 mg brexpiprazole once daily on each of the next 3 days following step (a);
(c) orally administering at least 4.125-7 mg brexpiprazole on each of the next 7 days following step (b); and then
(d) orally administering 1-2 mg brexpiprazole once daily thereafter;
wherein the obese patient has one or more of the following characteristics:
(i) BMI of at least about 35;
(ii) % IBW of at least about 150%;
(iii) waist size greater than about 42 inches;
(iv) % body fat greater than about 40%;
(v) % android body fat greater than about 40%;
(vi) % gynoid body fat greater than about 40%; or
(vii) total body fat greater than about 40 kg.

22-37. (canceled)

38. A method of initiating treatment of major depressive disorder with brexpiprazole in an obese patient who is not a CYP2D6 poor metabolizer, comprising:
(a) orally administering 0.625-0.875 mg brexpiprazole once daily on each of the first 7 days of brexpiprazole treatment;
(b) orally administering 1.125-1.875 mg brexpiprazole once daily on each of the next 7 days following step (a);
(c) orally administering 2-2.875 mg brexpiprazole once daily on each of the next 7 days step (b); and then
(d) orally administering a recommended dose of brexpiprazole of 2-3 mg once daily thereafter;
wherein the obese patient has one or more of the following characteristics:
(i) BMI of at least about 35;
(ii) % IBW of at least about 150%;
(iii) waist size greater than about 42 inches;
(iv) % body fat greater than about 40%;
(v) % android body fat greater than about 40%;
(vi) % gynoid body fat greater than about 40%; or
(vii) total body fat greater than about 40 kg.

39-53. (canceled)

54. A method of initiating treatment of major depressive disorder with brexpiprazole in an obese patient who is not a CYP2D6 poor metabolizer, comprising:
(a) orally administering 1.125-1.875 mg brexpiprazole once daily on each of the first 7 days of brexpiprazole treatment;
(b) orally administering 2.125-3.875 mg brexpiprazole once daily on each of the next 7 days following step (a);
(c) orally administering 2-4 mg brexpiprazole once daily on each of the next 7 days following step (b); and then
(d) orally administering a recommended dose of brexpiprazole of 2-3 mg once daily thereafter;
wherein the obese patient has one or more of the following characteristics:
(i) BMI of at least about 35;
(ii) % IBW of at least about 150%;
(iii) waist size greater than about 42 inches;
(iv) % body fat greater than about 40%;
(v) % android body fat greater than about 40%;
(vi) % gynoid body fat greater than about 40%; or
(vii) total body fat greater than about 40 kg.

55-68. (canceled)

69. A method of initiating treatment of major depressive disorder with brexpiprazole in an obese patient who is a CYP2D6 poor metabolizer, comprising:
(a) orally administering 0.625-0.875 mg brexpiprazole once daily on each of the first 7 days of brexpiprazole treatment;
(b) orally administering 1-1.875 mg brexpiprazole once daily on each of the next 7 days following step (a);
(c) orally administering 1-3 mg brexpiprazole once daily on each of the next 7 days following step (b); and then
(d) orally administering 1-1.5 mg once daily thereafter;
wherein the obese patient has one or more of the following characteristics:
(i) BMI of at least about 35;
(ii) % IBW of at least about 150%;
(iii) waist size greater than about 42 inches;
(iv) % body fat greater than about 40%;
(v) % android body fat greater than about 40%;
(vi) % gynoid body fat greater than about 40%; or
(vii) total body fat greater than about 40 kg.

70-83. (canceled)

84. A method of initiating treatment of major depressive disorder with brexpiprazole in an obese patient who is a CYP2D6 poor metabolizer, comprising:
(a) orally administering 1.125-1.875 mg brexpiprazole once daily on each of the first 7 days of brexpiprazole treatment;
(b) orally administering 2-3.5 mg brexpiprazole once daily on each of the next 7 days following step (a);
(c) orally administering 1-3 mg brexpiprazole once daily on each of the next 7 days following step (b); and then
(d) orally administering 1.1.5 mg brexpiprazole once daily thereafter;
wherein the obese patient has one or more of the following characteristics:
(i) BMI of at least about 35;
(ii) % IBW of at least about 150%;
(iii) waist size greater than about 42 inches;
(iv) % body fat greater than about 40%;
(v) % android body fat greater than about 40%;
(vi) % gynoid body fat greater than about 40%; or
(vii) total body fat greater than about 40 kg.

85-98. (canceled)

99. A method of initiating treatment of schizophrenia with brexpiprazole in a normal-weight patient who is a CYP2D6 poor metabolizer, comprising:
(a) administering 0.625-1.325 mg brexpiprazole once daily on each of the first 4 days of brexpiprazole treatment;
(b) administering 1.5-2 mg brexpiprazole once daily on each of the next 3 days following step (a); and then
(c) 2.5-3.5 mg brexpiprazole once daily on each of the next 3 days following step (b); and
then
(d) administering 1-2 mg brexpiprazole once daily thereafter,
wherein the normal-weight patient has at least one of the following characteristics:
(i) BMI less than about 35 kg/m 2;
(ii) % IBW less than about 150%;
(iii) waist size less than about 42 inches;
(iv) % body fat less than about 40%;
(v) % android body fat less than about 40%;
(vi) % gynoid body fat less than about 40%; or
(vii) total body fat less than about 40 kg.

100-113. (canceled)

114. A method of initiating adjunctive treatment of major depressive disorder with brexpiprazole in a normal-weight patient who is a CYP2D6 poor metabolizer, comprising:
(a) administering 0.375 mg brexpiprazole once daily on each of the first 7 days of brexpiprazole treatment;
(b) administering 0.625-0.875 mg of brexpiprazole once daily on each of the next 7 days following step (a);
(c) administering 1.125-1.75 mg of brexpiprazole once daily on each of the next 7 days following step (b);
(d) administering 1-1.5 mg of brexpiprazole once daily thereafter;
wherein the normal-weight patient has at least one of the following characteristics:
(i) BMI less than about 35 kg/m 2;
(ii) % IBW less than about 150%;
(iii) waist size less than about 42 inches;
(iv) % body fat less than about 40%;
(v) % android body fat less than about 40%;
(vi) % gynoid body fat less than about 40%; or
(vii) total body fat less than about 40 kg.

115-117. (canceled)

118. A method of initiating adjunctive treatment of major depressive disorder with brexpiprazole in a normal-weight patient who is a CYP2D6 poor metabolizer, comprising:
(a) administering 0.75 mg of brexpiprazole once daily on each of the first 7 days of brexpiprazole treatment;
(b) administering 1.25-2 mg of brexpiprazole once daily on each of the next 7 days following step (a);
(c) administering 1.5-3 mg of brexpiprazole once daily on each of the next 7 days following step (b);
(d) administering 1-1.5 mg of brexpiprazole once daily thereafter;
wherein the normal-weight patient has at least one of the following characteristics:
(i) BMI less than about 35 kg/m 2;
(ii) % IBW less than about 150%;
(iii) waist size less than about 42 inches;
(iv) % body fat less than about 40%;
(v) % android body fat less than about 40%;
(vi) % gynoid body fat less than about 40%; or
(vii) total body fat less than about 40 kg.

119-124. (canceled)
